(19) United States
(12) Patent Application Publication
(10) Pub. No.: US 2023/0372551 A1
(43) Pub. Date: Nov. 23, 2023

(54) RADIOLABELED LIGANDS FOR TARGETED PET/SPECT IMAGING AND METHODS OF THEIR USE

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE OF CANCER RESEARCH, New York, NY (US); UNIVERSITY OF RHODE ISLAND BOARD OF TRUSTEES, Kingston, RI (US); PHLIP, INC., Kingston, RI (US)

(72) Inventors: Jason S. LEWIS, New York, NY (US); Yana K. RESHETNYAK, Saunderson, RI (US); Lukas M. CARTER, New York, NY (US); David BAUER, New York, NY (US)

(73) Assignees: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US); UNIVERSITY OF RHODE ISLAND BOARD OF TRUSTEES, Kingston, RI (US); PHLIP, INC., Kingston, RI (US)

(21) Appl. No.: 18/027,337

(22) PCT Filed: Sep. 21, 2021
(86) PCT No.: PCT/US2021/051322

§ 371 Date: Mar. 20, 2023

Related U.S. Application Data
(60) Provisional application No. 63/081,632, filed on Sep. 22, 2020.
(60) Provisional application No. 63/242,315, filed on Sep. 9, 2021.

(51) Int. Cl. A61K 51/08; C07K 14/00

(52) U.S. Cl. CPC A61K 51/088; C07K 14/001; A61K 2123/00

(57) ABSTRACT

The present disclosure provides compounds, complexes, compositions, and methods for the detection of cancer. Specifically, the compounds, complexes, compositions of the present technology include pH (low) insertion peptides. Also disclosed herein are methods of using the complexes and compositions of the present technology in diagnostic imaging to detect cancer in a subject.

### CROSS-REFERENCE TO RELATED APPLICATIONS

This application claims the benefit of and priority to U.S. Provisional Appl. No. 63/081,632 filed Sep. 22, 2020 and to U.S. Provisional Appl. No. 63/242,315 filed Sep. 9, 2021, each of which is incorporated herein by reference in their entirety for any and all purposes.

### STATEMENT OF GOVERNMENT SUPPORT

This invention was made with government support under CA186721, CA138468, and CA008748, awarded by the National Institutes of Health. The government has certain rights in the invention.

### TECHNICAL FIELD

The present technology relates generally to compositions including a modified pH (low) insertion peptide and methods of using the same in diagnostic imaging to detect acidic tissues, such as cancer, in a subject.

### BACKGROUND

The following description of the background of the present technology is provided simply as an aid in understanding the present technology and is not admitted to describe or constitute prior art to the present technology.

Cancer cells generally prefer the glycolytic pathway of energy production due to their deregulated proliferative machinery and survival needs, thus resulting in excess acidity. To maintain homeostasis, cancer cells release lactic acid formed during glycolysis to the extracellular environment. Lactic acid release lowers the extracellular pH immediately surrounding these cells relative to the pH of normal tissues. Further, the production of carbon dioxide by the rapidly metabolizing cells causes expression of carbonic anhydrases on the cancer cell surfaces, promoting further acidification of the extracellular environment as the cells export the carbon dioxide and the anhydrases convert it into bicarbonate ions and protons. Due to the differential in pH gradients, a probe marking these acidic regions in rapidly proliferating tissues can potentially discriminate cancerous tissues from normal tissue.

Thus, there is a need for novel diagnostic compositions that exhibit (a) high tumoral uptake and prolonged retention in tumors, and (b) minimal accumulation in non-tumor tissue for use in PET and SPECT imaging methods to detect tumor cells.

### SUMMARY OF THE PRESENT TECHNOLOGY

In an aspect, the present disclosure provides a compound or pharmaceutically acceptable salt thereof, where the compound includes a pH (low) insertion peptide (“pHLIP”) configured to localize to an extracellular environment having a pH that is lower than 7.4, wherein the pHLIP comprises a C-terminus and an N-terminus; and X¹ covalently attached to a heteroatom of a side chain of an amino acid residue of the pHLIP, where the amino acid residue is from 0, 1, 2, 3, 4, 5, 6, 7, 8, 9, or 10 residues from the C-terminus or the N-terminus.

In the compound, X¹ is of Formula I, Formula II, or Formula III

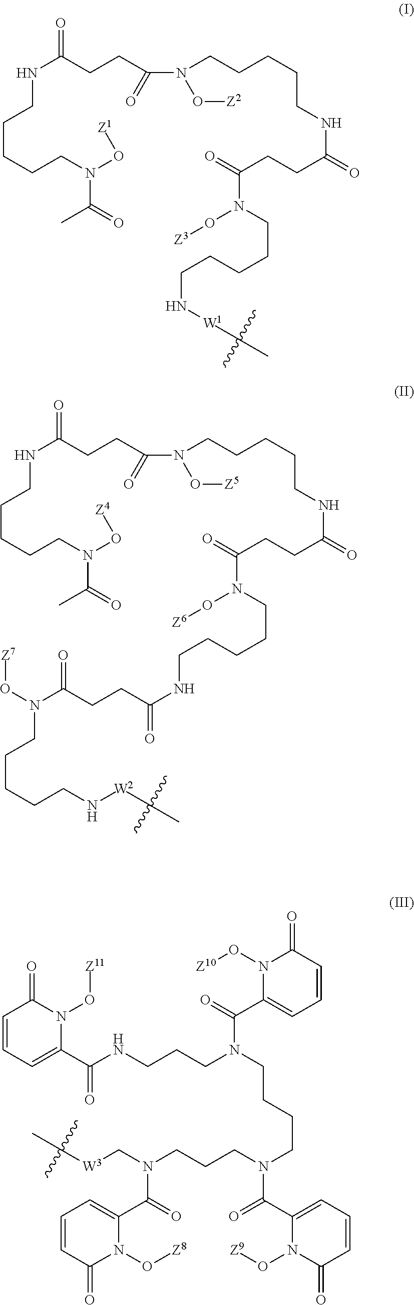

where Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; W¹ and W² are each independently

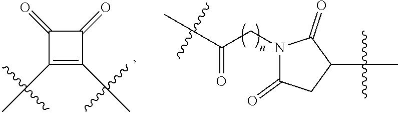

where n is 1, 2, 3, 4, 5, 6, 7, 8, 9, 10, 11, or 12,

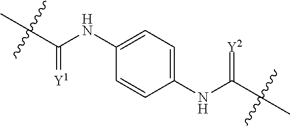

where Y¹ and Y² are each independently O or S, alkylene, alkanoylene, poly(alkylene glycol), —C(O)CH₂-(poly(alkylene glycol))-, or a bond, and W³ is

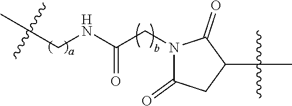

where a is 0, 1, 2, 3, 4, 5, 6, 7, 8, 9, 10, 11, or 12, and bis 1, 2, 3, 4, 5, 6, 7, 8, 9, 10, 11, or 12,

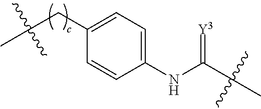

where Y³ is O or S, and c is 0, 1, 2, 3, 4, or 5,

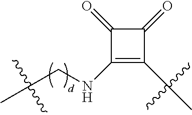

H where d is 0, 1, 2, 3, 4, 5, 6, 7, 8, 9, 10, 11, or 12,

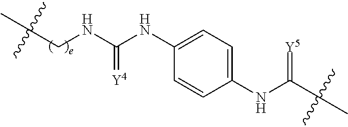

where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond. For example, in any embodiment herein the compound may be Ac-(D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Ala) (SEQ ID NO. 240) where X¹ is of Formula Ia

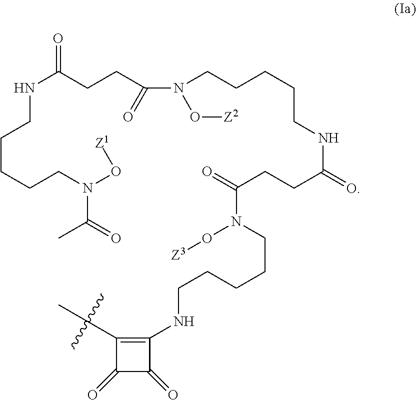

In a related aspect, a complex is provided that includes any embodiment of the compound described herein and a radionuclide. In such a complex, it may be that X² represents X¹ and the radionuclide together, where X² is represented by Formula IV, Formula V, or Formula VI

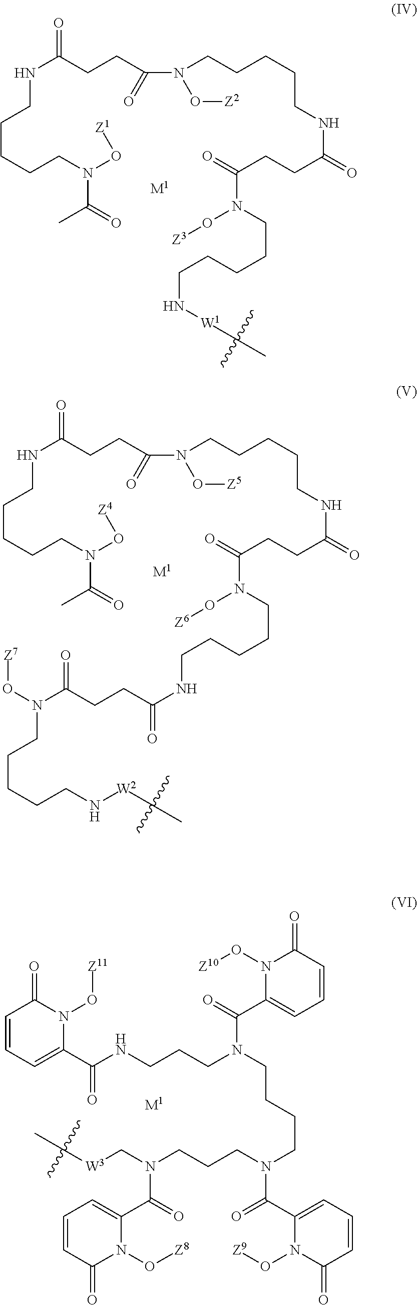

wherein M¹ is independently at each occurrence the radionuclide; Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and W¹ and W² are each independently

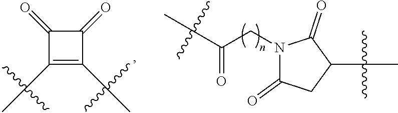

where n is 1, 2, 3, 4, 5, 6, 7, 8, 9, 10, 11, or 12,

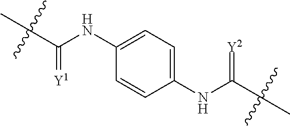

where Y¹ and Y² are each independently O or S, alkylene, alkanoylene, poly(alkylene glycol), —C(O)CH₂-(poly(alkylene glycol))-, or a bond, and W³ is

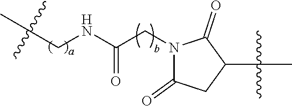

where a is 0, 1, 2, 3, 4, 5, 6, 7, 8, 9, 10, 11, or 12, and b is 1, 2, 3, 4, 5, 6, 7, 8, 9, 10, 11, or 12,

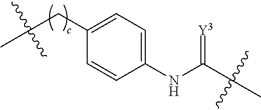

where Y³ is O or S, and c is 0, 1, 2, 3, 4, or 5,

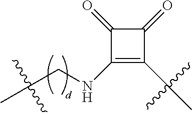

where d is 0, 1, 2, 3, 4, 5, 6, 7, 8, 9, 10, 11, or 12,

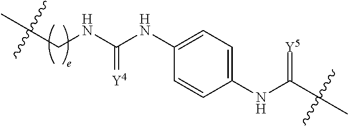

where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond. For example, in any embodiment herein the complex may be Ac-(D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D- Leu)-(D-Leu)-(D-Asp)-(D-Leu)-(D-Leu)-(D-Trp)-(D-Ala) (SEQ ID NO. 241) where X² is of Formula IVa

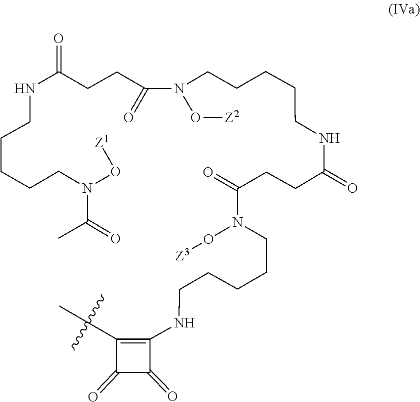

and M¹ is ⁸⁹Zr⁴⁺.

In another related aspect of the present technology, a composition is provided that includes any one of the aspects and embodiments of compounds and/or complexes and a pharmaceutically acceptable carrier. In a further related aspect, a pharmaceutical composition is provided, the pharmaceutical composition including an effective amount of any one of the embodiments of the complexes described herein for imaging a tissue comprising an extracellular environment having a pH that is lower than 7.4 and a pharmaceutically acceptable carrier.

In an aspect, the present disclosure provides a method for detecting solid tumors in a subject in need thereof comprising (a) administering to the subject an effective amount of a complex of any embodiment described herein, wherein the complex is configured to localize to a solid tumor having an acidic pH environment; and (b) detecting the presence of one or more solid tumors in the subject by detecting radioactive levels emitted by the complex that are higher than a reference value.

In another aspect, the present disclosure provides a method for detecting acidic diseased tissue in a subject in need thereof comprising (a) administering to the subject an effective amount of a complex of any embodiment described herein, wherein the complex is configured to localize to an extracellular environment having a pH that is lower than 7.4; and (b) detecting the presence of acidic diseased tissue in the subject by detecting radioactive levels emitted by the complex that are higher than a reference value.

Also disclosed herein are kits containing components suitable for diagnosing, e.g., cancer, in a patient. In one aspect, the kits comprise at least one compound of the present technology and instructions for use.

### BRIEF DESCRIPTION OF THE DRAWINGS

FIG. 1 provides the ex vivo biodistributions in selected organs (mean % ID/g SD) at the indicated time points post-injection of a complex of the present technology into mice bearing 4T1 tumors, according to the working examples.

FIG. 2 provides in vivo PET image slices at the level of 4T1 tumors for representative mice imaged at the indicated time points post-injection of a complex of the present technology, according to the working examples. The arrowheads indicate where the tumor is located in the mouse.

FIG. 3 provides representative in vivo PET images for mice bearing brain tumors at 4 hours post-intratumoral injection, 24 hours post-intratumoral injection, and 168 hours post-intratumoral injection, of a complex of the present technology via maximum intensity projection (“MIP”) and coronal slice (“Axial”) PET/CT images, according to the working examples.

FIG. 4 provides mean organ uptake levels as determined by volume-of-interest analysis of the in vivo PET image data indicated regarding FIG. 3, according to the working examples.

FIG. 5 provides tryptophan fluorescence spectra of a “cold” variant (“Zr-DFO-cys-Var3pHLIP”) of a complex of the present technology in aqueous solution at pH 8, at pH 8 in the presence of POPC liposomes, and at pH 4 in the presence of POPC liposomes, evidencing the interaction of Zr-DFO-cys-Var3pHLIP with a lipid bilayer of membrane, according to the working examples.

FIG. 6 provides circular dichroism spectra of Zr-DFO-cys-Var3pHLIP in aqueous solution at pH 8, at pH 8 in the presence of POPC liposomes, and at pH 4 in the presence of POPC liposomes, also evidencing the interaction of Zr-DFO-cys-Var3pHLIP with a lipid bilayer of membrane, according to the working examples

FIG. 7 illustrates the changes in the position of maxima of tryptophan fluorescence spectra as a function of pH evidencing the pH-dependent insertion of Zr-DFO-cys-Var3pHLIP into the lipid bilayers of POPC liposomes, according to the working examples. Data were fitted using the Henderson-Hlasselbalch equation; the fitting curves and 95% confidence interval are shown by the black line and the grey shading, respectively.

FIG. 8 shows a representative kinetic curve for the insertion of Zr-DFO-cys-Var3pHLIP into the lipid bilayer triggered by drops of pH from pH 8 to pH 4 as provided by the normalized fluorescence measured via 320 cut off filter, according to the working examples. The black line is a one-exponential fitting.

FIG. 9 illustrates the changes of the tryptophan fluorescence spectra for Zr-DFOsqa-lys-Var3pHLIP indicating insertion of Zr-DFOsqa-lys-Var3pHLIP into the lipid bilayers of POPC liposomes induced by drop of pH from 8 to 3, according to the working examples. The data were fitted with the Henderson-Hasselbach equation (black line with 95% confidence interval) to establish the midpoint of the transition (pK).

FIG. 10 illustrates the changes of the tryptophan fluorescence spectra for Zr-DFO*-lys-Var3pHLIP indicating insertion of Zr-DFO*-lys-Var3pHLIP into the lipid bilayers of POPC liposomes induced by drop of pH from 8 to 3, according to the working examples. The data were fitted with the Henderson-Hasselbach equation (black line with 95% confidence interval) to establish the midpoint of the transition (pK).

FIG. 11 illustrates the changes of the tryptophan fluorescence spectra for Zr-DFO-lys-Var3pHLIP indicating insertion of Zr-DFO-lys-Var3pHLIP into the lipid bilayers of POPC liposomes induced by drop of pH from 8 to 3, according to the working examples. The data were fitted with the Henderson-Hasselbach equation (black line with 95% confidence interval) to establish the midpoint of the transition (pK).

FIG. 12 illustrates the changes of the circular dichroism of Zr-DFOsqa-lys-Var3pHLIP measured in millidegree indicating on coil-helix transition induced by drop of pH from 8 to 3, where the data were fitted with the Henderson-Hasselbach equation (black line with 95% confidence interval) to establish the midpoint of the transition (pK), according the working examples.

FIG. 13 illustrates the changes of the circular dichroism of Zr-DFO*-lys-Var3pHLIP measured in millidegree indicating on coil-helix transition induced by drop of pH from 8 to 3, where the data were fitted with the Henderson-Hasselbach equation (black line with 95% confidence interval) to establish the midpoint of the transition (pK), according the working examples.

FIG. 14 illustrates the changes of the circular dichroism of Zr-DFO-lys-Var3pHLIP measured in millidegree indicating on coil-helix transition induced by drop of pH from 8 to 3, where the data were fitted with the Henderson-Hasselbach equation (black line with 95% confidence interval) to establish the midpoint of the transition (pK), according the working examples.

FIG. 15 graphically illustrates the ex vivo distribution in selected organs (mean % ID/g±standard deviation) at 48 p.i. for groups of male athymic nude mice (4 mice per group) bearing orthotopic RM-1 tumor allografts injected with 200 μCi (1.2 nmol) of a complex of the present technology, accoriding to the working examples.

FIG. 16 provides coronal in vivo PET images (maximum intensity projection) at 48 h p.i. for complexes of the present technology, according to the working examples, where the circles indicate the tumor's location (scale: percent injected dose per gram of tissue (% ID/g)).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The present disclosure provides compounds, complexes, and compositions that include modified pH (low) insertion peptides that exhibit high tumoral uptake, prolonged tumor retention, and minimal accumulation in non-target organs and tissues. These complexes and compositions are useful as diagnostic imaging agents because they permit diagnostic imaging over a wider-range of times, rather than shorter time points (e.g., fludeoxyglucose F 18 (“¹⁸F-FDG”), a radiopharmaceutical used in the medical imaging modality positron emission tomography) or much longer time points (e.g., radiolabeled antibodies) in order to detect acidic tissues, such as cancerous cells.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry, biochemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term (e.g., except where such number would be less than 0% or exceed 100% of a possible value)—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, intralesionally, or subcutaneously), rectally, topically, intraarterially, intrathecally, or via inhalation or via introduction into the cerebrospinal fluid. Administration includes self-administration and the administration by another.

As used herein, the term “amino acid” includes naturally-occurring amino acids and synthetic amino acids, as well as amino acid analogues that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Amino acids can be referred to herein by either their commonly known three letter symbols or by the one-letter symbols recommended by the IUPAC-IUB Biochemical Nomenclature Commission.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. In any embodiment herein, the peptides included in the compounds and complexes of the present technology may include only D-amino acids.

As used herein, the term “cancer” refers to a malignant neoplasm or tumor (Stedman's Medical Dictionary, 25th ed.; Hensly ed.; Williams & Wilkins: Philadelphia, 1990). Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CML, T cell CML), and chronic lymphocytic leukemia (CLL) (e.g., B cell CLL, T cell CLL)); lymphoma such as Hodgkin lymphoma (HL) (e.g., B cell HL, T cell HL) and non Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenström's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, a “control” is an alternative sample used in an experiment for comparison purpose. A control can be “positive” or “negative.”

As used herein, “radiolabel” refers to a moiety comprising a radioactive isotope of at least one element. Exemplary suitable radiolabels include but are not limited to those described herein. In some embodiments, a radiolabel is one used in positron emission tomography (PET). In some embodiments, a radiolabel is one used in single-photon emission computed tomography (SPECT).

As used herein, the term “sample” refers to clinical samples obtained from a subject or isolated microorganisms. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the terms “subject,” “individual,” or “patient” are used interchangeably and refer to an individual organism, a vertebrate, a mammal, or a human. In certain embodiments, the individual, patient or subject is a human.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

The terms “alkanoyl” and “alkanoyloxy” as used herein can refer, respectively, to —C(O)-alkyl groups and —O—C(O)-alkyl groups.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein refers to —OH.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, trifluoroacetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and alkaline earth metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

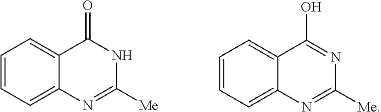 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

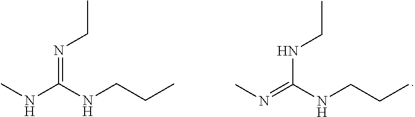

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry

### Compositions of the Present Technology

pH (low) insertion peptides represent a unique class of delivery agents that can target acidic malignant tissue. Without being bound by theory, the molecular mechanism of targeting is based on the pH-dependent formation of a transmembrane alpha helix, which is accompanied by the insertion of pH (low) insertion peptides into the cellular membrane in environments with relatively low extracellular pH. There are at least three folding states of pH (low) insertion peptides: unfolded and unbound peptide in solution (State I), unfolded peptide loosely interacting with the membrane lipid bilayer at physiological pH (State II), and folded peptide in alpha-helical conformation inserted across the membrane at low extracellular pH (State III).

In an aspect, a compound or pharmaceutically acceptable salt thereof is provided, where the compound includes a pH (low) insertion peptide (“pHLIP”) configured to localize to an extracellular environment having a pH that is lower than 7.4, wherein the pHLIP comprises a C-terminus and an N-terminus; and X¹ covalently attached to a heteroatom of a side chain of an amino acid residue of the pHLIP, where the amino acid residue is from 0, 1, 2, 3, 4, 5, 6, 7, 8, 9, or 10 residues from the C-terminus or the N-terminus. An amino acid residue that is 0 residues from the N-terminus will be understood to mean the amino acid residue is the N-terminal residue. Similarly, an amino acid residue that is 0 residues from the C-terminus will be understood to mean the amino acid residue is the C-terminal residue. The extracellular environment may have a pH that is lower than 7.1. For example, the extracellular environment may have a pH that is 7.1, 7.0, 6.9, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0, 1.8, 1.6, 1.4, 1.2, 1.0, or any range including and/or in between any two of these values. A tissue (e.g., in a subject) may include the extracellular environment. Such tissues include, but are not limited to, atherosclerotic plaques, ischemic myocardium, tissues impacted by stroke, inflamed tissues, and cancer tissues (such as solid tumors). Exemplary cancer tissues include, but are not limited to, breast cancer, colorectal cancer, cervical cancer, ovarian cancer, liver cancer, bladder cancer, hepatoma, hepatocellular carcinoma, brain tumors, lung cancer, gastric or stomach cancer, pancreatic cancer, thyroid cancer, kidney or renal cancer, prostate cancer, melanoma, sarcomas, carcinomas, Wilms tumor, endometrial cancer, glioblastoma, squamous cell cancer, astrocytomas, salivary gland carcinoma, vulvar cancer, penile carcinoma, and head-and-neck cancer; the cancer tissue may be breast cancer, a brain tumor, prostate cancer, melanoma, or a metastatic cancer thereof.

In the compound, X¹ is of Formula I, Formula II, or Formula III

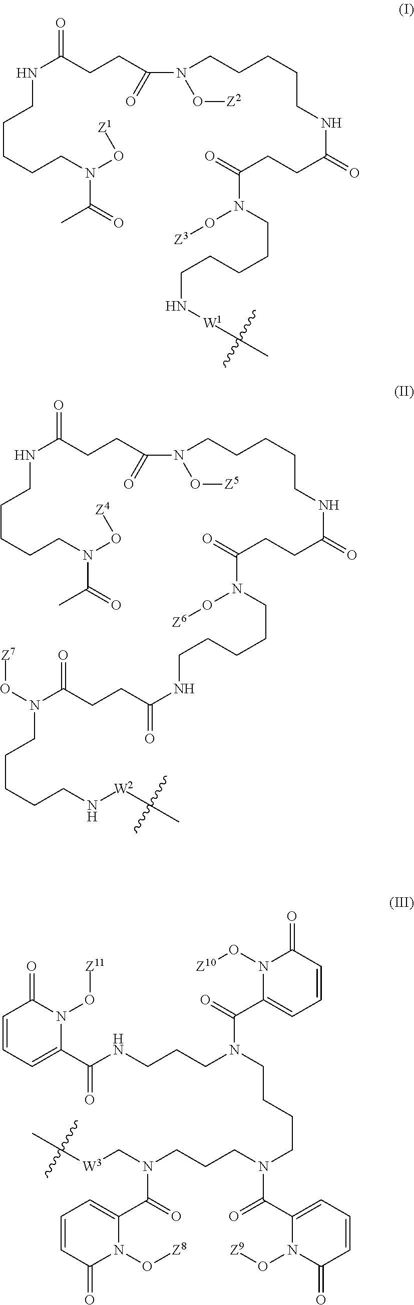

where Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; W¹ and W² are each independently

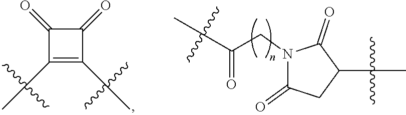

where n is 1, 2, 3, 4, 5, 6, 7, 8, 9, 10, 11, or 12,

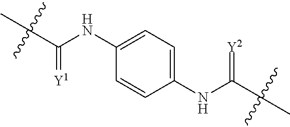

where Y¹ and Y² are each independently O or S, alkylene, alkanoylene, poly(alkylene glycol), —C(O)CH₂-(poly(alkylene glycol))-, or a bond, and W³ is

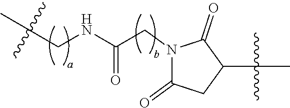

where a is 0, 1, 2, 3, 4, 5, 6, 7, 8, 9, 10, 11, or 12, and b is 1, 2, 3, 4, 5, 6, 7, 8, 9, 10, 11, or 12,

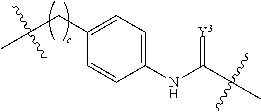

where Y³ is O or S, and c is 0, 1, 2, 3, 4, or 5,

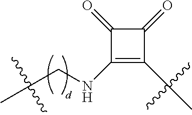

where d is 0, 1, 2, 3, 4, 5, 6, 7, 8, 9, 10, 11, or 12,

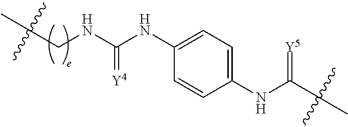

where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond. In any embodiment disclosed herein, it may be W¹ and W² are each independently

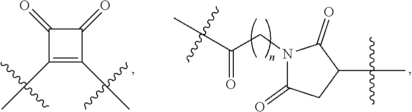

—(CH₂)ₘ—, —C(O)(CH₂)ₚ—, —CH₂CH₂—(O—CH₂CH₂)_{q}—, —C(O)CH₂—(O—CH₂CH₂)ᵣ—, or a bond; where n is 1, 2, 3, 4, 5, 6, 7, 8, 9, 10, 11, or 12; m is 1, 2, 3, 4, 5, 6, 7, 8, 9, 10, 11, or 12; p is 1, 2, 3, 4, 5, 6, 7, 8, 9, 10, 11, or 12; q is 1, 2, 3, 4, 5, 6, 7, 8, 9, 10, 11, or 12; and r is 1, 2, 3, 4, 5, 6, 7, 8, 9, 10, 11, or 12.
In any embodiment disclosed herein, it may be W³ is

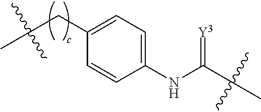

where Y³ is O or S, and c is 0, 1, 2, 3, 4, or 5,

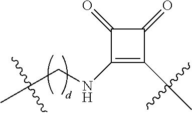

where d is 0, 1, 2, 3, 4, 5, 6, 7, 8, 9, 10, 11, or 12, —(CH₂)_{f}—, —C(O)(CH₂)_{g}—, —CH₂CH₂—(O—CH₂CH₂)ₕ—, —C(O)CH₂—(O—CH₂CH₂)ᵢ—, or a bond, where f is 1, 2, 3, 4, 5, 6, 7, 8, 9, 10, 11, or 12, g is 1, 2, 3, 4, 5, 6, 7, 8, 9, 10, 11, or 12, h is 1, 2, 3, 4, 5, 6, 7, 8, 9, 10, 11, or 12, and i is 1, 2, 3, 4, 5, 6, 7, 8, 9, 10, 11, or 12. In any embodiment herein, the X¹ of Formula I may be of Formula Ia, of Formula Ib, or of Formula IIIa

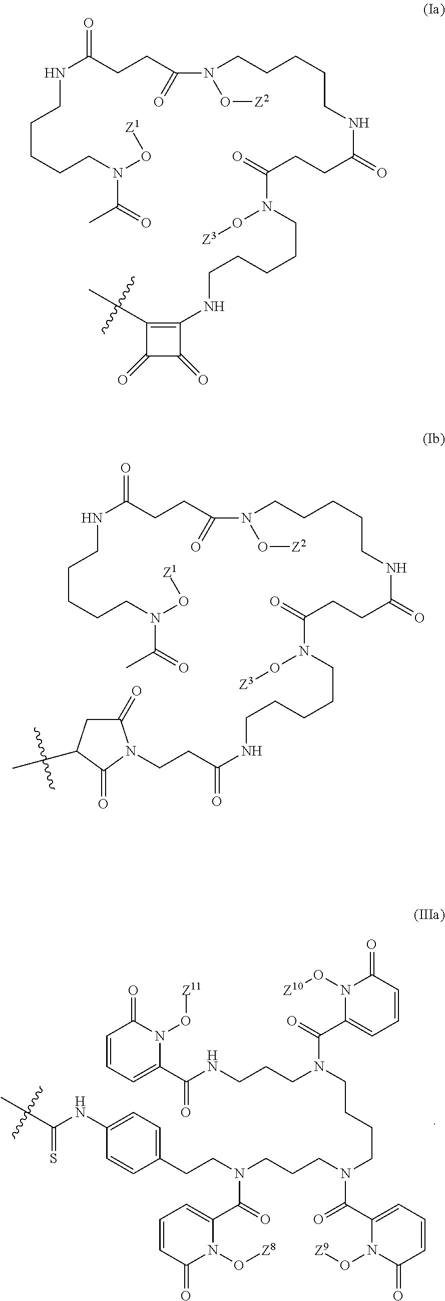

The pHLIP may be any one described in U.S. Pat. Nos. 8,076,451, 8,703,909, 8,846,081, 9,676,823, and 9,289,508, each of which are incorporated herein by reference for any and all purposes, as well as U.S. Patent Application Nos. 2015/0086617 and 2016/0256560, each of which are incorporated herein by reference for any and all purposes. The amino acid residue of the pHLIP (to which heteroatom of the side chain X¹ is covalently attached) may be a cysteine or lysine. In any embodiment herein, the X¹ may be covalently attached to a sulfur atom of a cysteine residue of the pHLIP or may be covalently attached to a ε-nitrogen atom of a lysine residue the pHLIP. For sake a clarity, a representation of the amino acid L-lysine is provided below indicating the ε-nitrogen atom.

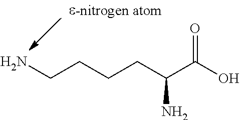

In any embodiment herein, the X¹ may be covalently attached to a sulfur atom of a D-cysteine residue of the pHLIP or may be covalently attached to a F-nitrogen atom of a D-lysine residue the pHLIP. In any embodiment herein, the pHLIP may be
Ac-(D-Ala)-(D-Lys)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D- Ala) (SEQ ID NO. 239) [note: “Ac” means acetylated N-terminus],
(D-Ala)-(D-Cys)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp) (SEQ ID NO. 1),
(D-Ala)-(D-Cys)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp) (SEQ ID NO. 2),
(D-Ala)-(D-Lys)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp) (SEQ ID NO. 3),
(D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp) (SEQ ID NO. 4),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys)-(Gly) (SEQ ID NO. 5),
(D-Ala)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys)-(Gly) (SEQ ID NO. 6),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys)-(Gly) (SEQ ID NO. 7),
(D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys)- (Gly) (SEQ ID NO. 8), or
an N-acetylated variant of any one of SEQ ID NO. 1, SEQ ID NO. 2, SEQ ID NO. 3, SEQ ID NO. 4, SEQ ID NO. 5, SEQ ID NO. 6, SEQ ID NO. 7, and SEQ ID NO. 8.

The pHLIP may, in any embodiment herein, be one of the exemplary pHLIP as disclosed via Tables 1-7 below. For Tables 4-7, the sequences refer to all L-amino acids, at least one L-amino acid and one or more D-amino acids, or all D-amino acids, where each D residue or E residue (see Tables 1-3) may be replaced by its substitution from Table 2; each non-polar residue may be replaced by its coded amino acid substitution from Table 3, and/or non-coded amino acid substitutions from Table 3. Further, the pHLIP in any embodiment heren may be an N-acetylated variant of a non-N-acetylated sequence disclosed in Tables 4-7.

TABLE 1
Coded and exemplary non-coded amino acids including L-isomers,
D-isomers, alpha-isomers, beta-isomers, glycol-, and methyl-
modifications referred to in Tables 2-7.

| Abbreviation | Name |
|---|---|
| Ala | Alanine |
| Arg | Arginine |
| Asn | Asparagine |
| Asp | Aspartic acid |
| Cys | Cysteine |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Phe | Phenylalanine |
| Pro | Proline |
| Ser | Serine |
| Thr | Threonine |
| Trp | Tryptophan |
| Tyr | Tyrosine |
| Val | Valine |
| Sec | Selenocysteine |
| Sem | Selenomethionine |
| Pyl | Pyrrolysine |
| Aad | Alpha-aminoadipic acid |
| Acpa | Amino-caprylic acid |
| Aecys | Aminoethyl cysteine |
| Afa | Aminophenyl acetate |
| Gaba | Gamma-aminobutyric acid |
| Aiba | Aminoisobutyric acid |
| Aile | Alloisoleucine |
| AIg | Allylglycine |
| Aba | Amino-butyric acid |
| Aphe | Amino-phenylalanine |
| Brphe | Bromo-phenylalanine |
| Cha | Cyclo-hexylalanine |
| Cit | Citrulline |
| Clala | Chloroalanine |
| Cie | Cycloleucine |
| Clphe | Fenclonine (or chlorophenylalanine) |
| Cya | Cysteic acid |
| Dab | Diaminobutyric acid |
| Dap | Diaminopropionic acid |
| Dap | Diaminopimelic acid |
| Dhp | Dehydro-proline |
| Dhphe | DOPA (or 3,4-dihydroxyphenylalanine) |
| Fphe | Fluorophenylalanine |
| Gaa | Glucosaminic acid |
| Gla | Gamma-carboxyglutamic acid |
| Hag | Homoarginine |
| Hlys | Hydroxylysine |
| Hnvl | Hydroxynorvaline |
| Hog | Homoglutamine |
| Hoph | Homophenylalanine |
| Has | Homoserine |
| Hse | Homocysteine |
| Hpr | Hydroxyproline |
| Iphe | Iodo-phenylalanine |
| Ise | Isoserine |
| Mle | Methyl-leucine |
| Msmet | Methionine-methylsulfonium chloride |
| Nala | Naphthyl-alanine |
| Nle | Norleucine (or 2-aminohexanoic acid) |
| Nmala | N-methyl-alanine |
| Nva | Norvaline (or 2-aminopentanoic acid) |
| Obser | O-benzyl-serine |
| Obtyr | O-benzyl-tyrosine |
| Oetyr | O-ethyl-tyrosine |
| Omser | O-methyl-serine |
| Omthr | O-methy-threonine |
| Omtyr | O-methyl-tyrosine |
| Orn | Ornithine |
| Pen | Penicillamine |
| Pga | Pyroglutamic acid |
| Pip | Pipecolic acid |
| Sa | Sarcosine |
| Tfa | Trifluoroalanine |
| Thphe | Hydroxy-Dopa |
| Vig | Vinylglycine |
| Aaspa | Amino-aminoethylsulfanylpropanoic acid |
| Ahdna | Amino-hydroxy-dioxanonanolic acid |
| Ahoha | Amino-hydroxy-oxahexanoic acid |
| Ahsopa | Amino-hydroxyethylsulfanylpropanoic acid |
| Tyr(Me) | Methoxyphenyl-methylpropanyl oxycarbonylamino |
| | propanoic acid |
| MTrp | Methyl-tryptophan |
| pTyr | Phosphorylated Tyr |
| pSer | Phosphorylated Ser |
| pThr | Phosphorylated Thr |
| BLys | BiotinLys |
| Hyp | Hydroproline |
| Phg | Phenylglycine |
| Cha | Cyclohexyl-alanine |
| Chg | Cyclohexylglycine |
| Nal | Naphthylalanine |
| Pal | Pyridyl-alanine |
| Pra | Propargylglycine |
| Gly(allyl) | Pentenoic acid |
| Pen | Penicillamine |
| MetO | Methionine sulfoxide |
| Pca | Pyroglutamic acid |
| Ac-Lys | Acetylated Lys |

TABLE 2
Non-limiting examples of certain residues and their substitutions
including L-isomers, D- isomers, alpha-isomers, and beta-isomers.

| Original Residue | Exemplary amino acid substitution |
|---|---|
| Asp (D) | Glu (E); Gla (Gla); Aad (Aad) |
| Glu (E) | Asp (D); Gla (Gla); Aad (Aad) |

TABLE 3
Examples of coded amino acid substitutions

| Original Residue | Substitution |
|---|---|
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

TABLE 4
Exemplary pHLIP

| Ref. | Sequence |
|---|---|
| Var3-1a | ADQDNPWRAYLDLLFPTDTLLLDLLWCA (SEQ ID NO. 26) |
| Var3-1b | ADQDNPWRAYLDLLFPTDTLLLDLLWKA (SEQ. ID NO. 27) |
| WT-1 | GGEQNPIYWARYADWLFTTPLLLLDLALLVDADEGT (SEQ. ID NO. 28) |
| WT-2 | AEQNPIYWARYADWLFTTPLLLLDLALLVDADEGT (SEQ. ID NO. 29) |
| Var3-CK | ADDQNPWRAYLDLLFPTDTLLLDLLWCKA (SEQ. ID NO. 30) |
| Var3-WT- | ADDQNPWRAYLDLLFPTDTLLLDLLWDADECG (SEQ. ID NO. 31) |
| Cys | |
| Var3-WT- | ADDQNPWRAYLDLLFPTDTLLLDLLWDADEKG (SEQ. ID NO. 32) |
| Lys | |
| Var3-WT- | ADDQNPWRAYLDLLFPTDTLLLDLLWDADEKCG (SEQ. ID NO. 33) |
| KC | |
| Cys-Var3- | ACDDQNPWRAYLDLLFPTDTLLLDLLWDADEG (SEQ. ID NO. 34) |
| WT | |
| Lys-Var3- | AKDDQNPWRAYLDLLFPTDTLLLDLLWDADEG (SEQ. ID NO. 35) |
| WT | |
| WT-Cys1 | AAEQNPIYWARYADWLFTTPLLLLDLALLVDADEGTCG (SEQ. ID NO. 36) |
| WT-Cys2 | Ac-AEQNPIYWARYADWLFTTPLLLLDLALLVDADEGCT (SEQ. ID NO. 37) |
| WT-Cys3 | GGEQNPIYWARYADWLFTTPLLLLDLALLVDADEGTCG (SEQ. ID NO. 38) |
| Cys-WT1 | Ac-ACEQNPIYWARYADWLFTTPLLLLDLALLVDADEGTG (SEQ. ID NO. |
| | 39) |
| Var0-NT | ACEQNPIYWARYADWLFTTPLLLLDLALLVDADEGT (SEQ. ID NO. 40) |
| Lys-WT1 | AKEQNPIYWARYADWLFTTPLLLLDLALLVDADEGT (SEQ. ID NO. 41) |
| Lys-WT2 | Ac-AKEQNPIYWARYADWLFTTPLLLLDLALLVDADEGTG (SEQ. ID NO. |
| | 42) |
| WT-KC | AAEQNPIYWARYADWLFTTPLLLLDLALLVDADEGTKCG (SEQ. ID NO. |
| | 43) |
| K-WT-C | AKEQNPIYWARYADWLFTTPLLLLDLALLVDADECT (SEQ. ID NO. 44) |
| N-pHLIP | ACEQNPIYWARYANWLFTTPLLLLNLALLVDADEGTG (SEQ. ID NO. 45) |
| N-pHLIP-b | ACEQNPIYWARYANWLFTTPLLLLNLALLVDADEGT (SEQ. ID NO. 46) |
| K-pHLIP | ACEQNPIYWARYAKWLFTTPLLLLKLALLVDADEGTG (SEQ. ID NO. 47) |
| NNQ | GGEQNPIYWARYADWLFTTPLLLLDLALLVNANQGT (SEQ. ID NO. 48) |
| D25A | AAEQNPIYWARYADWLFTTPLLLLALALLVDADEGT (SEQ. ID NO. 49) |
| D25A-KC | Ac-AAEQNPIYWARYADWLFTTPLLLLELALLVDADEGTKCG (SEQ. ID |
| | NO. 50) |
| D14A | AAEQNPIYWARYAAWLFTTPLLLLDLALLVDADEGT (SEQ. ID NO. 51) |
| P20A | AAEQNPIYWARYADWLFTTALLLLDLALLVDADEGT (SEQ. ID NO. 52) |
| D25E | AAEQNPIYWARYADWLFTTPLLLLELALLVDADEGT (SEQ. ID NO. 53) |
| D14E | AAEQNPIYWARYAEWLFTTPLLLLDLALLVDADEGT (SEQ. ID NO. 54) |
| 3D | AAEQNPITYWARYADWLFTDLPLLLLDLLALLVDADEGT (SEQ. ID NO. 55) |
| R11Q | GEQNPIYWAQYADWLFTTPLLLLDLALLVDADEGTCG (SEQ. ID NO. 56) |
| D25Up | GGEQNPIYWARYADWLFTTPLLLDLLALLVDADEGTCG (SEQ. ID NO. 57) |
| D25Down | GGEQNPIYWARYADWLFTTPLLLLLDALLVDADEGTCG (SEQ. ID NO. 58) |
| D14Up | GGEQNPIYWARYDAWLFTTPLLLLDLALLVDADEGTCG (SEQ. ID NO. 59) |
| D14Down | GGEQNPIYWARYAWDLFTTPLLLLDLALLVDADEGTCG (SEQ. ID NO. 60) |
| P20G | AAEQNPIYWARYADWLFTTGLLLLDLALLVDADEGT (SEQ. ID NO. 61) |
| H1-Cys | DDDEDNPIYWARYADWLFTTPLLLLHGALLVDADECT (SEQ. ID NO. 62) |
| H1 | DDDEDNPIYWARYADWLFTTPLLLLHGALLVDADET (SEQ. ID NO. 63) |
| H2-Cys | DDDEDNPIYWARYAHWLFTTPLLLLHGALLVDADEGCT (SEQ. ID NO. 64) |
| Cys-H2 | CDDDEDNPIYWARYAHWLFTTPLLLLHGALLVDADET (SEQ. ID NO. 65) |
| H2 | DDDEDNPIYWARYAHWLFTTPLLLLHGALLVDADEGT (SEQ. ID NO. 66) |
| H2N-Cys | DDDEDNPIYWARYAHWLFTTPLLLLHGALLVNADECT (SEQ. ID NO. 67) |
| H2N | DDDEDNPIYWARYAHWLFTTPLLLLHGALLVNADEGT (SEQ. ID NO. 68) |
| H2N2-Cys | DDDEDNPIYWARYAHWLFTTPLLLLHGALLVNANECT (SEQ. ID NO. 69) |
| H2N2 | DDDEDNPIYWARYAHWLFTTPLLLLHGALLVNANEGT (SEQ. ID NO. 70) |
| 1a-Trp | AEQNPIYWARYADFLFTTPLLLLDLALLVDADET (SEQ. ID NO. 71) |
| 1b-Trp | AEQNPIYFARYADWLFTTPLLLLDLALLVDADEGT (SEQ. ID NO. 72) |
| 1c-Trp | AEQNPIYFARYADFLFTTPLLLLDLALLWDADET (SEQ. ID NO. 73) |
| Fast-1 or | AKEDQNPYWARYADWLFTTPLLLLDLALLVDG (SEQ. ID NO. 74) |
| Var1 | |
| Var1-2D1D | ACEDQNPYWARYADWLFTTPLLLLDLALLVDG (SEQ. ID NO. 75) |
| Fast1-Cys | AEDQNPYWARYADWLFTTPLLLLDLALLVDCG (SEQ. ID NO. 76) |
| or Var1- | |
| 2D1D-Cys | |
| Fast1-E-Cys | AEDQNPYWARYADWLFTTPLLLLELALLVECG (SEQ. ID NO. 77) |
| or Var1E | |
| Fast1-E-Lys | AKEDQNDPYWARYADWLFTTPLLLLDLALLVG (SEQ. ID NO. 78) |
| Fast2 or | AKEDQNPYWRAYADLFTPLTLLDLLALWDG (SEQ. ID NO. 79) |
| Var2 | |
| Fast2-E-Cys | AEDQNPYWARYADWLFTTPLLLLELALLVCG (SEQ. ID NO. 80) |
| or Var2E | |
| Var2-2D1D | ACEDQNPYWRAYADLFTPLTLLDLLALWDG (SEQ. ID NO. 81) |
| Var3-3D | ACDDQNPWRAYLDLLFPTDTLLLDLLW (SEQ. ID NO. 82) |
| Var3-3D- | AKDDQNPWRAYLDLLFPTDTLLLDLLWC (SEQ. ID NO. 83) |
| cys | |
| Var4-3E | ACEEQNPWRAYLELLFPTETLLLELLW (SEQ. ID NO. 84) |
| Var5-3Da | ACDDQNPWARYLDWLFPTDTLLLDL (SEQ. ID NO. 85) |
| Var6-3Db | CDNNNPWRAYLDLLFPTDTLLLDW (SEQ. ID NO. 86) |
| Var8-3Eb | CEEQQPWAQYLELLFPTETLLLEW (SEQ. ID NO. 87) |
| Var9-3Ec | CEEQQPWRAYLELLFPTETLLLEW (SEQ. ID NO. 88) |
| Var15-2N | CDDDDDNPNYWARYANWLFTTPLLLLNGALLVEAEET (SEQ. ID NO. 89) |
| Var16-2P | CDDDDDNPNYWARYAPWLFTTPLLLLPGALLVEAEE (SEQ. ID NO. 90) |

TABLE 5
Exemplary pHLIP (cont.)

| Ref. | Sequence |
|---|---|
| Var14- | Ac-TEDADVLLALDLLLLPTTFLWDAYRAWYPNQECA-Am |
| Rev | (SEQ. ID NO. 91) |
| Sh | AEQNPIYWARYADWLFTTPL (SEQ. ID NO. 92) |
| Sh-Cys | AEQNPIYWARYADWLFTTPCL (SEQ. ID NO. 93) |
| Cys-Sh | ACEQNPIYWARYADWLFTTPL (SEQ. ID NO. 94) |
| Sh-1Trp | AEQNPIYFARYADWLFTTPL (SEQ. ID NO. 95) |
| Sh-W2 | AEQNPIYFARYADLLFPTTLAW (SEQ. ID NO. 96) |
| Sh-W1 | AEQNPIYWARYADLLFPTTLAF (SEQ. ID NO. 97) |
| Sh-2W | AEQNPIYWARYADLLFPTTLAW (SEQ. ID NO. 98) |
| Sh-1D | KEDQNPWARYADLLFPTTLAW (SEQ. ID NO. 99) |
| Sh-1Db | KEDQNPWARYADLLFPTTLW (SEQ. ID NO. 100) |
| Var12-1D | ACEDQNPWARYADLLFPTTLAW (SEQ. ID NO. 101) |
| Var10-2D | ACEDQNPWARYADWLFPTTLLLLD (SEQ. ID NO. |
| | 102) |
| Var13-1E | ACEEQNPWARYAELLFPTTLAW (SEQ. ID NO. 103) |
| Var11-2E | ACEEQNPWARYAEWLFPTTLLLLE (SEQ. ID NO. |
| | 104) |
| Var7-3E | ACEEQNPWARYLEWLFPTETLLLEL (SEQ. ID NO. |
| | 105) |
| Var7-3Eb | ACEEQNPQAEYAEWLFPTTLLLLE (SEQ. ID NO. |
| | 106) |

“Ac” means acetylated N-terminus
“Am” means amidated C-terminus

TABLE 6
Non-limiting examples of membrane-inserting sequences
belonging to different groups of pHLIPs.

| Group | Sequence |
|---|---|
| WT-BRC | WARYADWLFTTPLLLLDLALL (SEQ. ID NO. 107) |
| | YARYADWLFTTPLLLLDLALL (SEQ. ID NO. 108) |
| | WARYSDWLFTTPLLLYDLGLL (SEQ. ID NO. 109) |
| | WARYTDWFTTPLLLYDLALLA (SEQ. ID NO. 110) |
| | WARYTDWLFTTPLLLYDLGLL (SEQ. ID NO. 111) |
| | WARYADWLFTTPLLLLDLSLL (SEQ. ID NO. 112) |
| WT-BRC Reverse | LLALDLLLLPTTFLWDAYRAW (SEQ. ID NO. 113) |
| | LLALDLLLLPTTFLWDAYRAY (SEQ. ID NO. 114) |
| | LLGLDYLLLPTTFLWDSYRAW (SEQ. ID NO. 115) |
| | ALLALDYLLLPTTFWDTYRAW (SEQ. ID NO. 116) |
| | LLGLDYLLLPTTFLWDTYRAW (SEQ. ID NO. 117) |
| | LLSLDLLLLPTTFLWDAYRAW (SEQ. ID NO. 118) |
| ATRAM | GLAGLLGLEGLLGLPLGLLEGLWLGL (SEQ. ID NO. |
| | 119) |
| ATRAM Reverse | LGLWLGELLGLPLGLLGELGLLGALG (SEQ. ID NO. |
| | 120) |
| Var3 | WRAYLDLLFPTDTLLLDLLW (SEQ. ID NO. 121) |
| Var3 Reverse | WLLDLLLTDTPFLLDLYARW (SEQ. ID NO. 122) |
| Var7 | WARYLEWLFPTETLLLEL (SEQ. ID NO. 123) |
| | WAQYLELLFPTETLLLEW (SEQ. ID NO. 124) |
| Var7 Reverse | LELLLTETPFLWELYRAW (SEQ. ID NO. 125) |
| | WELLLTETPFLLELYQAW (SEQ. ID NO. 126) |
| Single D/E | WLFTTPLLLLNGALLVE (SEQ. ID NO. 127) |
| | WLFTTPLLLLPGALLVE (SEQ. ID NO. 128) |
| | WARYADLLFPTTLAW (SEQ. ID NO. 129) |
| Single D/E Reverse | EVLLAGNLLLLPTTFLW (SEQ. ID NO. 130) |
| | EVLLAGPLLLLPTTFLW (SEQ. ID NO. 131) |
| | WALTTPFLLDAYRAW (SEQ. ID NO. 132) |
| pHLIP-Rho | NLEGFFATLGGEIALWSLVVLAIE (SEQ. ID NO. 133) |
| | EGFFATLGGEIALWSDVVLAIE (SEQ. ID NO. 134) |
| | EGFFATLGGEIPLWSDVVLAIE (SEQ. ID NO. 135) |
| pHLIP-Rho Reverse | EIALVVLSWLAIEGGLTAFFGELN (SEQ. ID NO. 136) |
| | EIALVVDSWLAIEGGLTAFFGE (SEQ. ID NO. 137) |
| | EIALVVDSWLPIEGGLTAFFGE (SEQ. ID NO. 138) |
| PHILIP-CA9 | ILDLVFGLLFAVTSVDFLVQW (SEQ. ID NO. 139) |
| pHLIP-CA9 Reverse | WQVLFDVSTVAFLLGFVLDLI (SEQ. ID NO. 140) |

TABLE 7
Further exemplary pHLIP.

| Ref. | Sequence |
|---|---|
| WT-2D | AEQNPIYWARYADWLFTTPLLLLDLALLVDADET (SEQ. ID NO. 141) |
| WT-6E | AEQNPIYWARYAEWLFTTPLLLLELALLVEAEET (SEQ. ID NO. 142) |
| WT-3D | ADDQNPWRAYLDLLFPDTTDLLLLDLLWDADET (SEQ. ID NO. 143) |
| WT-9E | AEEQNPWRAYLELLFPETTELLLLELLWEAEET (SEQ. ID NO. 144) |
| WT-GlaD | AEQNPIYWARYAGlaWLFTTPLLLLDLALLVDADET (SEQ. ID NO. 145) |
| WT-DGla | AEQNPIYWARYADWLFTTPLLLLGlaLALLVDADET (SEQ. ID NO. 146) |
| WT-2Gla | AEQNPIYWARYAGlaWLFTTPLLLLGlaLALLVDADET (SEQ. ID NO. 147) |
| WT-AadD | AEQNPIYWARYAAadWLFTTPLLLLDLALLVDADET (SEQ. ID NO. 148) |
| WT-DAad | AEQNPIYWARYADWLFTTPLLLLAadLALLVDADET (SEQ. ID NO. 149) |
| WT-2Aad | AEQNPIYWARYAAadWLFTTPLLLLAadLALLVDADET (SEQ. ID NO. 150) |
| WT- | AEQNPIYWARYAGlaWLFTTPLLLLAadLALLVDADET (SEQ. ID NO. 151) |
| GlaAad | |
| WT- | AEQNPIYWARYAAadWLFTTPLLLLGlaLALLVDADET (SEQ. ID NO. 152) |
| AadGla | |
| WT-1 | GGEQNPIYWARYADWLFTTPLLLLDLALLVDADEGT (SEQ. ID NO. 153) |
| WT-2 | GGEQNPIYWARYADWLFTTPLLLLDLALLVDADEGT (SEQ. ID NO. 154) |
| WT-3 | AAEQNPIYWARYADWLFTTPLLLLDLALLVDADEGT (SEQ. ID NO. 155) |
| WT-4 | AEQNPIYWARYADWLFTTPLLLLDLALLVDADEGT (SEQ. ID NO. 156) |
| WT-2N | AEQNPIYWARYANWLFTTPLLLLNLALLVDADEGT (SEQ. ID NO. 157) |
| WT-2K | AEQNPIYWARYAKWLFTTPLLLLKLALLVDADEGT (SEQ. ID NO. 158) |
| WT- | GGEQNPIYWARYADWLFTTPLLLLDLALLVNANQGT (SEQ. ID NO. 159) |
| 2DNANQ | |
| WT-D25A | AAEQNPIYWARYADWLFTTPLLLLALALLVDADEGT (SEQ. ID NO. 160) |
| WT-D14A | AAEQNPIYWARYAAWLFTTPLLLLDLALLVDADEGT (SEQ. ID NO. 161) |
| WT-P20A | AAEQNPIYWARYADWLFTTALLLLDLALLVDADEGT (SEQ. ID NO. 162) |
| WT-D25E | AAEQNPIYWARYADWLFTTPLLLLELALLVDADEGT (SEQ. ID NO. 163) |
| WT-D14E | AAEQNPIYWARYAEWLFTTPLLLLDLALLVDADEGT (SEQ. ID NO. 164) |
| WT-3D-2 | AAEQNPITYWARYADWLFTDLPLLLLDLLALLVDADEGT (SEQ. ID NO. 165) |
| WT-R11Q | GEQNPIYWAQYADWLFTTPLLLLDLALLVDADEG (SEQ. ID NO. 166) |
| WT- | GGEQNPIYWARYADWLFTTPLLLDLLALLVDADEG (SEQ. ID NO. 167) |
| D25Up | |
| WT- | GGEQNPIYWARYADWLFTTPLLLLLDALLVDADEG (SEQ. ID NO. 169) |
| D25Down | |
| WT- | GGEQNPIYWARYDAWLFTTPLLLLDLALLVDADEGT (SEQ. ID NO. 170) |
| D14Up | |
| WT- | GGEQNPIYWARYAWDLFTTPLLLLDLALLVDADEG (SEQ. ID NO. 171) |
| D14Down | |
| WT-P20G | AAEQNPIYWARYADWLFTTGLLLLDLALLVDADEGT (SEQ. ID NO. 172) |
| WT-DH | DDDEDNPIYWARYADWLFTTPLLLLHGALLVDAD (SEQ. ID NO. 173) |
| WT-2H | DDDEDNPIYWARYAHWLFTTPLLLLHGALLVDADE (SEQ. ID NO. 174) |
| WT-L16H | CEQNPIYWARYADWHFTTPLLLLDLALLVDADE (SEQ. ID NO. 175) |
| WT-1Wa | AEQNPIYWARYADFLFTTPLLLLDLALLVDADET (SEQ. ID NO. 176) |
| WT-1Wb | AEQNPIYFARYADWLFTTPLLLLDLALLVDADE (SEQ. ID NO. 177) |
| WT-1Wc | AEQNPIYFARYADFLFTTPLLLLDLALLWDADET (SEQ. ID NO. 178) |
| WT-W6 | ADNNPWIYARYADLTTFPLLLLDLALLVDFDD (SEQ. ID NO. 179) |
| WT-W17 | ADNNPFIYARYADLTTWPLLLLDLALLVDFDD (SEQ. ID NO. 180) |
| WT-W30 | ADNNPFIYARYADLTTFPLLLLDLALLVDWDD (SEQ. ID NO. 181) |
| WT-W17- | ADNNPFPYARYADLTTWILLLLDLALLVDFDD (SEQ. ID NO. 182) |
| P7 | |
| WT-W39- | ADNNPFIYAYRADLTTFPLLLLDLALLVDWDD (SEQ. ID NO. 183) |
| R11 | |
| WT-W30- | ADNNPFIYATYADLRTFPLLLLDLALLVDWDD (SEQ. ID NO. 184) |
| R15 | |
| WT-Rev | Ac-TEDADVLLALDLLLLPTTFLWDAYRAWYPNQEA-Am (SEQ. ID NO. 185) |
| Var1-3D | AEDQNPYWARYADWLFTTPLLLLDLALLVD (SEQ. ID NO. 186) |
| Var1-1D2E | AEDQNPYWARYADWLFTTPLLLLELALLVE (SEQ. ID NO. 187) |
| Var2-3D | AEDQNPYWRAYADLFTPLTLLDLLALWD (SEQ. ID NO. 188) |
| Var3-3D | ADDQNPWRAYLDLLFPTDTLLLDLLW (SEQ. ID NO. 189) |
| Var3-WT | ADDQNPWRAYLDLLFPTDTLLLDLLWDADE (SEQ. ID NO. 190) |
| Var3- | ADDQNPWRAYLGlaLLFPTDTLLLDLLW (SEQ. ID NO. 191) |
| Gla2D | |
| Var3- | ADDQNPWRAYLDLLFPTG/aTLLLDLLW (SEQ. ID NO. 192) |
| DGlaD | |
| Var3- | ADDQNPWRAYLDLLFPTDTLLLG/aLLW (SEQ. ID NO. 193) |
| 2DGla | |
| Var3- | ADDQNPWRAYLGlaLLFPTGlaTLLLDLLW (SEQ. ID NO. 194) |
| 2GlaD | |
| Var3- | ADDQNPWRAYLGlaLLFPTDTLLLGlaLLW (SEQ. ID NO. 195) |
| GlaDGla | |
| Var3- | ADDQNPWRAYLDLLFPTGlaTLLLGlaLLW (SEQ. ID NO. 196) |
| D2Gla | |
| Var3-3Gla | ADDQNPWRAYLGlaLLFPTGlaTLLLGlaLLW (SEQ. ID NO. 197) |
| Var3- | ADDQNPWRAYLAadLLFPTDTLLLDLLW (SEQ. ID NO. 198) |
| Aad2D | |
| Var3- | ADDQNPWRAYLDLLFPTAadTLLLDLLW (SEQ. ID NO. 199) |
| DAadD | |
| Var3- | ADDQNPWRAYLDLLFPTDTLLLAadLLW (SEQ. ID NO. 200) |
| 2DAad | |
| Var3- | ADDQNPWRAYLAadLLFPTAadTLLLDLLW (SEQ. ID NO. 201) |
| 2AadD | |
| Var3- | ADDQNPWRAYLAadLLFPTDTLLLAadLLW (SEQ. ID NO. 202) |
| AadDAad | |
| Var3- | ADDQNPWRAYLDLLFPTAadTLLLAadLLW (SEQ. ID NO. 203) |
| D2Aad | |
| Var3-3Aad | ADDQNPWRAYLAadLLFPTAadTLLLAadLLW (SEQ. ID NO. 204) |
| Var3- | ADDQNPWRAYLGlaLLFPTAadTLLLDLLW (SEQ. ID NO. 205) |
| GlaAadD | |
| Var3- | ADDQNPWRAYLGlaLLFPTDTLLLAadLLW (SEQ. ID NO. 206) |
| GlaDAad | |
| Var3- | ADDQNPWRAYLGlaLLFPTG/aTLLLAadLLW (SEQ. ID NO. 207) |
| 2GlaAad | |
| Var3- | ADDQNPWRAYLAadLLFPTGlaTLLLDLLW (SEQ. ID NO. 208) |
| AadGlaD | |
| Var3- | ADDQNPWRAYLAadLLFPTDTLLLGlaLLW (SEQ. ID NO. 209) |
| AadDGla | |
| Var3- | ADDQNPWRAYLGlaLLFPTAadTLLLGlaLLW (SEQ. ID NO. 210) |
| GlaAadGla | |
| Var3-GLL | GEEQNPWLGAYLDLLFPLELLGLLELGLW (SEQ. ID NO. 211) |
| Var3-M | ADDDDDDPWQAYLDLLFPTDTLLLDLLW (SEQ. ID NO. 212) |
| Var4-3E | AEEQNPWRAYLELLFPTETLLLELLW (SEQ. ID NO. 213) |
| Var5-3Da | ADDQNPWARYLDWLFPTDTLLLDL (SEQ. ID NO. 214) |
| Var6-3Db | DNNNPWRAYLDLLFPTDTLLLDW (SEQ. ID NO. 215) |
| Var7-3E | AEEQNPWARYLEWLFPTETLLLEL (SEQ. ID NO. 216) |
| Var7-M | DDDDDDPWQAYLDLFPTDTLALDLW (SEQ. ID NO. 217) |
| Var8-3E | EEQQPWAQYLELLFPTETLLLEW (SEQ. ID NO. 218) |
| Var9-3E | EEQQPWRAYLELLFPTETLLLEW (SEQ. ID NO. 219) |
| Var10-2D | AEDQNPWARYADWLFPTTLLLLD (SEQ. ID NO. 220) |
| Var11-2E | AEEQNPWARYAEWLFPTTLLLLE (SEQ. ID NO. 221) |
| Var12-1D | AEDQNPWARYADLLFPTTLAW (SEQ. ID NO. 222) |
| Var13-1E | AEEQNPWARYAELLFPTTLAW (SEQ. ID NO. 223) |
| Var15-2N | DDDDDNPNYWARYANWLFTTPLLLLNGALLVEAEET (SEQ. ID NO. 224) |
| Var16-2P | DDDDDNPNYWARYAPWLFTTPLLLLPGALLVEAEET (SEQ. ID NO. 225) |
| Var17 | AEQNPIYFARYADFLFTTPLLLLDLALLWDADET (SEQ. ID NO. 226) |
| Var18 | AEQNPIYWARYADFLFTTPLLLLDLALLVDADET (SEQ. ID NO. 227) |
| Var19a | AEQNPIYWARYADWLFTTPL (SEQ. ID NO. 228) |
| Var20 | AEQNPIYFARYADLLFPTTLAW (SEQ. ID NO. 229) |
| Var21 | AEQNPIYWARYADLLFPTTLAF (SEQ. ID NO. 230) |
| Var22 | AEQNPIYWARYADLLFPTTLAW (SEQ. ID NO. 231) |
| Var23 | AEQNPIYFARYADWLFTTPL (SEQ. ID NO. 232) |
| Var24 | EDQNPWARYADLLFPTTLAW (SEQ. ID NO. 233) |
| ATRAM | GLAGLAGLLGLEGLLGLPLGLLEGLWLGLELEGN (SEQ. ID NO. 234) |
| PHLIP- | EQNPIYILDLVFGLLFAVTSVDFLVQWDDAGD (SEQ. ID NO. 235) |
| CA9 | |
| pHLIP-Rho | NLEGFFATLGGEIALWSLVVLAIE (SEQ. ID NO. 236) |
| pHLIP- | NNEGFFATLGGEIALWSDVVLAIE (SEQ. ID NO. 237) |
| RhoM1 | |
| pHLIP- | DNNEGFFATLGGEIPLWSDVVLAIE (SEQ. ID NO. 238) |
| RhoM2 | |

In any embodiment herein, the compound of the present technology may be

(SEQ ID NO. 240)
Ac-(D-Ala)-(D-Lys[X¹])-(D-Asp)-(D-Asp)-(D-Gln)-(D-Asn)-(D-Pro)-(D-Trp)-(D-
Arg)-(D-Ala)-(D-Tyr)-(D-Leu)-(D-Asp)-(D-Leu)-(D-Leu)-(D-Phe)-(D-Pro)-
(D-Thr)-(D-Asp)-(D-Thr)-(D-Leu)-(D-Leu)-(D-Leu)-(D-Asp)-(D-Leu)-(D-
Leu)-(D-Trp)-(D-Ala),
(SEQ ID NO. 9)
(D-Ala)-(D-Cys[X¹])-(D-Asp)-(D-Asp)-(D-Gln)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 10)
(D-Ala)-(D-Cys[X¹])-(D-Asp)-(D-Gln)-(D-Asp)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 11)
(D-Ala)-(D-Lys[X¹])-(D-Asp)-(D-Asp)-(D-Gln)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 12)
(D-Ala)-(D-Lys[X¹])-(D-Asp)-(D-Gln)-(D-Asn)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 13)
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys[X¹])-(Gly),
(SEQ ID NO. 14)
(D-Ala)-(D-Asp)-(D-Gln)-(D-Asp)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys[X¹])-(Gly),
(SEQ ID NO. 15)
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Lys[X¹])-(Gly),
(SEQ ID NO. 16)
(D-Ala)-(D-Lys)-(D-Asp)-(D-Gln)-(D-Asn)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp)-(D-Lys[X¹])-(Gly),

or a pharmaceutically acceptable salt of any one of these.

In a related aspect, a complex is provided that includes any embodiment of the compound described herein and a radionuclide. In such a complex, it may be that X² represents X¹ and the radionuclide together, where X² is represented by Formula IV, Formula V, or Formula VI

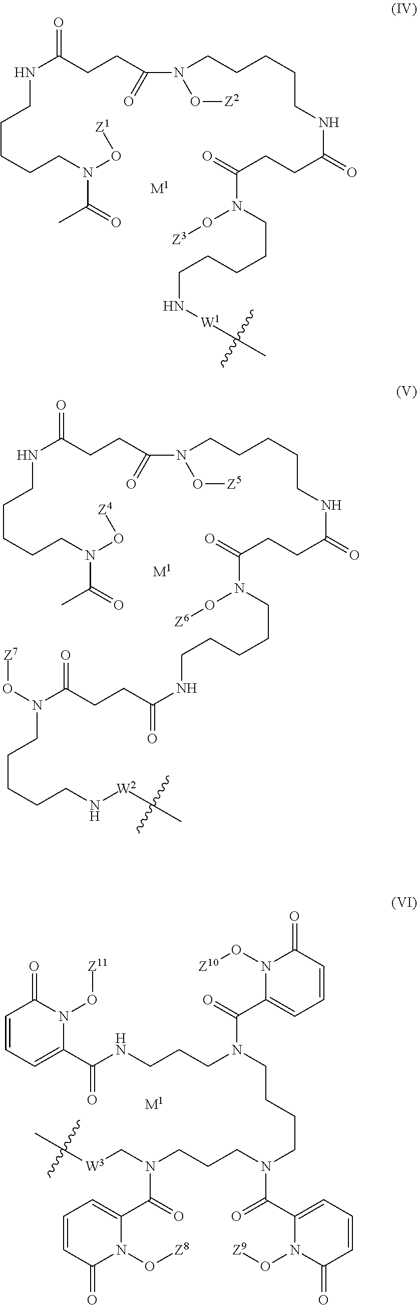

wherein M¹ is independently at each occurrence the radionuclide; Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and W¹ and W² are each independently

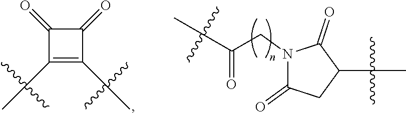

where n is 1, 2, 3, 4, 5, 6, 7, 8, 9, 10, 11, or 12,

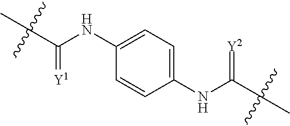

where Y¹ and Y² are each independently O or S, alkylene, alkanoylene, poly(alkylene glycol), —C(O)CH₂-(poly(alkylene glycol))-, or a bond, and W³ is

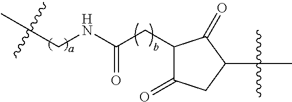

where a is 0, 1, 2, 3, 4, 5, 6, 7, 8, 9, 10, 11, or 12, and b is 1, 2, 3, 4, 5, 6, 7, 8, 9, 10, 11, or 12,

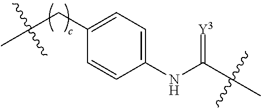

where Y³ is O or S, and c is 0, 1, 2, 3, 4, or 5,

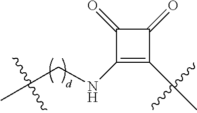

where d is 0, 1, 2, 3, 4, 5, 6, 7, 8, 9, 10, 11, or 12,

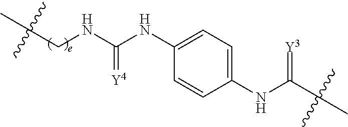

where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond. In any embodiment disclosed herein, it may be W¹ and W² are each independently

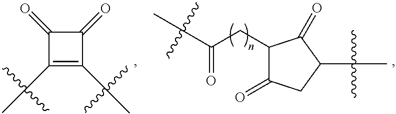

—(CH₂)ₘ—, —C(O)(CH₂)ₚ—, —CH₂CH₂—(O—CH₂CH₂)_{q}—, —C(O)CH₂—(O—CH₂CH₂)ᵣ—, or a bond; where n is 1, 2, 3, 4, 5, 6, 7, 8, 9, 10, 11, or 12; m is 1, 2, 3, 4, 5, 6, 7, 8, 9, 10, 11, or 12; p is 1, 2, 3, 4, 5, 6, 7, 8, 9, 10, 11, or 12; q is 1, 2, 3, 4, 5, 6, 7, 8, 9, 10, 11, or 12; and r is 1, 2, 3, 4, 5, 6, 7, 8, 9, 10, 11, or 12. In any embodiment disclosed herein, it may be W³ is

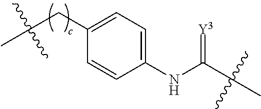

where Y³ is O or S, and c is 0, 1, 2, 3, 4, or 5,

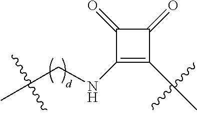

where d is 0, 1, 2, 3, 4, 5, 6, 7, 8, 9, 10, 11, or 12, —(CH₂)_{f}—, —C(O)(CH₂)_{g}—, —CH₂CH₂—(O—CH₂CH₂)ₕ—, —C(O)CH₂—(O—CH₂CH₂)ᵢ—, or a bond, where f is 1, 2, 3, 4, 5, 6, 7, 8, 9, 10, 11, or 12, g is 1, 2, 3, 4, 5, 6, 7, 8, 9, 10, 11, or 12, h is 1, 2, 3, 4, 5, 6, 7, 8, 9, 10, 11, or 12, and i is 1, 2, 3, 4, 5, 6, 7, 8, 9, 10, 11, or 12.

In any embodiment herein, X² may be of Formula IVa, of Formula IVb, or of Formula VIa

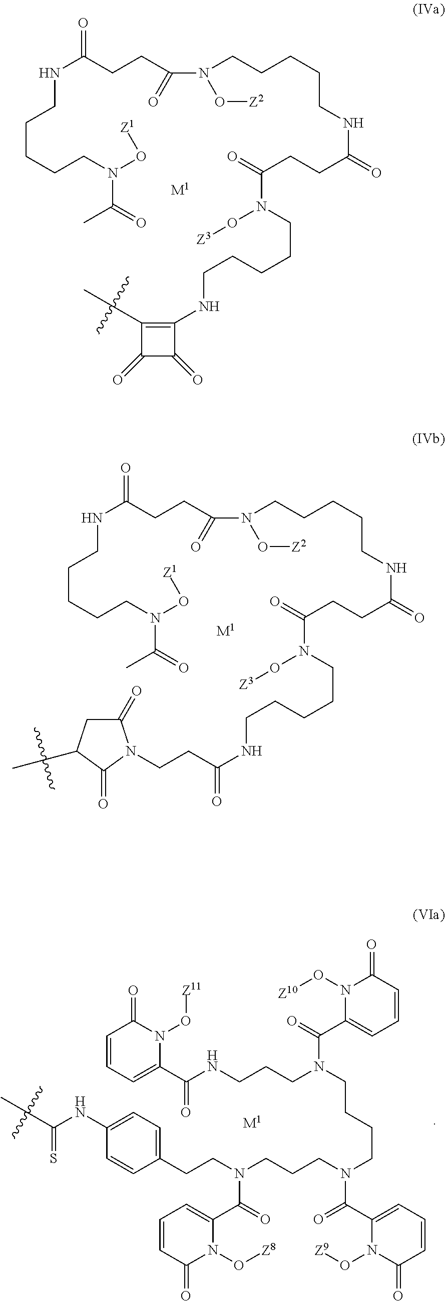

any embodiment herein, the complex of the present technology may be

(SEQ ID NO. 241)
Ac-(D-Ala)-(D-Lys[X²])-(D-Asp)-(D-Asp)-(D-Gln)-(D-Asn)-(D-Pro)-(D-Trp)-(D-
Arg)-(D-Ala)-(D-Tyr)-(D-Leu)-(D-Asp)-(D-Leu)-(D-Leu)-(D-Phe)-(D-Pro)-
(D-Thr)-(D-Asp)-(D-Thr)-(D-Leu)-(D-Leu)-(D-Leu)-(D-Asp)-(D-Leu)-(D-
Leu)-(D-Trp)-(D-Ala),
(SEQ ID NO. 17)
(D-Ala)-(D-Cys[X²])-(D-Asp)-(D-Asp)-(D-Gln)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 18)
(D-Ala)-(D-Cys[X²])-(D-Asp)-(D-Gln)-(D-Asp)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 19)
(D-Ala)-(D-Lys[X²])-(D-Asp)-(D-Asp)-(D-Gln)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 20)
(D-Ala)-(D-Lys[X²])-(D-Asp)-(D-Gln)-(D-Asn)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp),
(SEQ ID NO. 21)
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys[X²])-(Gly),
(SEQ ID NO. 22)
(D-Ala)-(D-Asp)-(D-Gln)-(D-Asp)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys[X²])-(Gly),
(SEQ ID NO. 23)
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Lys[X²])-(Gly),
(SEQ ID NO. 24)
(D-Ala)-(D-Lys)-(D-Asp)-(D-Gln)-(D-Asn)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp)-(D-Lys[X²])-(Gly),

or a pharmaceutically acceptable salt of any one of these. In any embodiment disclosed herein, the radionuclide may be ⁸⁹Zr⁴⁺, ⁶⁷Ga³⁺, or ⁶⁸Ga³⁺.

In another related aspect of the present technology, a composition is provided that includes any one of the aspects and embodiments of compounds and/or complexes and a pharmaceutically acceptable carrier or one or more excipients or fillers (collectively, such carriers, excipients, fillers, etc., will be referred to as “pharmaceutically acceptable carriers” unless a more specific term is used). In a further related aspect, a pharmaceutical composition is provided, the pharmaceutical composition including an effective amount of any one of the embodiments of the complexes described herein for imaging a tissue comprising an extracellular environment having a pH that is lower than 7.4 (or any value or range disclosed herein) and a pharmaceutically acceptable carrier. The tissue may be an atherosclerotic plaque, an ischemic myocardium, a tissue impacted by stroke, inflamed tissue, and/or a cancer tissue (such as a solid tumor). Exemplary cancer tissues (and exemplary solid tumors of such tissues) include, but are not limited to, breast cancer, colorectal cancer, cervical cancer, ovarian cancer, liver cancer, bladder cancer, hepatoma, hepatocellular carcinoma, brain tumors, lung cancer, gastric or stomach cancer, pancreatic cancer, thyroid cancer, kidney or renal cancer, prostate cancer, melanoma, sarcomas, carcinomas, Wilms tumor, endometrial cancer, glioblastoma, squamous cell cancer, astrocytomas, salivary gland carcinoma, vulvar cancer, penile carcinoma, and head-and-neck cancer; the cancer tissue may be breast cancer, a brain tumor, prostate cancer, melanoma, or a metastatic cancer thereof. Such compositions and pharmaceutical compositions may be used in any method described herein.

“Effective amount” refers to the amount of a complex required to produce a desired effect, such as a quantity of a complex of the present technology necessary to be detected by the detection method chosen. For example, an effective amount of a complex of the present technology includes an amount sufficient to enable detection of binding of the complex to a target of interest including, but not limited to, one or more of an atherosclerotic plaque, an ischemic myocardium, a tissue impacted by stroke, inflamed tissue, and/or a cancer tissue (such as a solid tumor). Another example of an effective amount includes amounts or dosages that are capable of providing a detectable positron emission and/or gamma ray emission from positron emission and annihilation (above background) in a subject with a tissue comprising an extracellular environment having a pH that is lower than 7.4, such as, for example, statistically significant emission above background. As used herein, a “subject” or “patient” is a mammal, such as a cat, dog, rodent or primate. Typically the subject is a human, and, preferably, a human suffering from or suspected of suffering from a condition that includes a tissue including an extracellular environment having a pH that is lower than 7.4 as described herein. The term “subject” and “patient” may be used interchangeably.

Thus, the instant present technology provides pharmaceutical compositions and medicaments comprising any of the compounds or complexes disclosed herein and a pharmaceutically acceptable carrier. The compositions may be used in the methods and imagings described herein. Such compositions and medicaments include an effective amount of any complex as described herein for imaging one or more of the herein-described tissues. The pharmaceutical composition may be packaged in unit dosage form. For example, the unit dosage form is effective in imaging a tissue including an extracellular environment having a pH that is lower than 7.4 when administered to a subject.

The pharmaceutical compositions and medicaments may be prepared by mixing one or more compounds or complexes of the present technology, pharmaceutically acceptable salts thereof, stereoisomers thereof, tautomers thereof, or solvates thereof, with pharmaceutically acceptable carriers, excipients, binders, diluents or the like to image disorders associated with a tissue that includes an extracellular environment having a pH that is lower than 7.4 when administered to a subject. The compounds and complexes described herein may be used to prepare formulations and medicaments for imaging a variety of disorders associated with a tissue comprising an extracellular environment having a pH that is lower than 7.4. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds or complexes of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. An isotonic solution will be understood as isotonic with the subject. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Complexes of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular complex, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of complexes of the present technology by inhalation.

Dosage forms for the topical (including buccal and sublingual) or transdermal administration of complexes of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds and complexes of the present technology across the skin. The rate of such flux can be controlled by either providing a rate controlling membrane (e.g., as part of a transdermal patch) or dispersing the compound and/or complex in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), and “Remington: The Science and Practice of Pharmacy,” 20^{th} Edition, Editor: Alfonso R Gennaro, Lippincott, Williams & Wilkins, Baltimore (2000), each of which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a complex of the present technology to a patient in increasing amounts until, for example, statistically significant resolution (via, e.g., positron emission tomography) of a tissue comprising an extracellular environment having a pH that is lower than 7.4 is achieved. The complexes of the present technology may be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being imaged, and the pharmacological activity of the complex being used. The determination of optimum dosages for a particular patient is well known to those skilled in the art. Various assays and model systems can be readily employed to determine the effectiveness of a complex according to the present technology.

The complexes of the present technology can also be administered to a patient along with other conventional imaging agents that may be useful in the imaging of a tissue that includes an extracellular environment having a pH that is lower than 7.4. Such tissues include, but are not limited to, an atherosclerotic plaque, an ischemic myocardium, a tissue impacted by stroke, inflamed tissue, and/or a cancer tissue (such as a solid tumor). Exemplary cancer tissues (and exemplary solid tumors of such tissues) include, but are not limited to, breast cancer, colorectal cancer, cervical cancer, ovarian cancer, liver cancer, bladder cancer, hepatoma, hepatocellular carcinoma, brain tumors, lung cancer, gastric or stomach cancer, pancreatic cancer, thyroid cancer, kidney or renal cancer, prostate cancer, melanoma, sarcomas, carcinomas, Wilms tumor, endometrial cancer, glioblastoma, squamous cell cancer, astrocytomas, salivary gland carcinoma, vulvar cancer, penile carcinoma, and head-and-neck cancer; the cancer tissue may be breast cancer, a brain tumor, prostate cancer, melanoma, or a metastatic cancer thereof. Thus, a pharmaceutical composition of the present technology may further include an imaging agent different than the complex of the present technology. The administration may include oral administration, parenteral administration, or nasal administration. In any of these embodiments, the administration may be intravenously, intramuscularly, intraarterially, intrathecally, intracapsularly, intradermally, intraperitoneally, intralesionally, subcutaneously, intracerebroventricularly, orally, intranasally, rectally, topically, or via inhalation. The methods of the present technology may also include administering, either sequentially or in combination with one or more complexes of the present technology, a conventional imaging agent in an amount that can potentially or synergistically be effective for the imaging of a tissue comprising an extracellular environment having a pH that is lower than 7.4.

In an aspect, a complex of the present technology is administered to a patient in an amount or dosage suitable for imaging. Generally, a unit dosage comprising a complex of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a complex of the present technology can vary from 1×10⁻⁴ g/kg to 1 g/kg, preferably, 1×10⁻³ g/kg to 1.0 g/kg. Dosage of a complex of the present technology can also vary from 0.01 mg/kg to 100 mg/kg or, preferably, from 0.1 mg/kg to 10 mg/kg.

The terms “associated” and/or “binding” can mean a chemical or physical interaction, for example, between a compound or complex of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, complexes of the present technology can bind to or interact with a target of interest or precursors, portions, fragments and peptides thereof and/or their deposits.

### Diagnostic Methods of the Present Technology

In one aspect, the present disclosure provides a method for detecting solid tumors in a subject in need thereof comprising (a) administering to the subject an effective amount of a complex of any embodiment described herein, wherein the complex is configured to localize to a solid tumor having an acidic pH environment (that is, an extracellular environment having a pH that is lower than 7.4); and (b) detecting the presence of one or more solid tumors in the subject by detecting radioactive levels emitted by the complex that are higher than a reference value. In some embodiments, the subject is human. In some embodiments, the environment of the solid tumor has a pH value that is 7.1, 7.0, 6.9, 6.8, 6.7, 6.6, 6.5, 6.4, 6.3, 6.2, 6.1, 6.0 or lower. For example, the extracellular environment of the solid tumor may have a pH that is 7.1, 7.0, 6.9, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0, 1.8, 1.6, 1.4, 1.2, 1.0, or any range including and/or in between any two of these values.

In some embodiments of the methods disclosed herein, the radioactive levels emitted by the complex are detected using positron emission tomography or single photon emission computed tomography.

Additionally or alternatively, in some embodiments of the methods disclosed herein, the subject is diagnosed with, is at risk for, or is suspected of having cancer. The cancer may be selected from the group consisting of breast cancer, colorectal cancer, cervical cancer, ovarian cancer, liver cancer, bladder cancer, hepatoma, hepatocellular carcinoma, brain tumors, lung cancer, gastric or stomach cancer, pancreatic cancer, thyroid cancer, kidney or renal cancer, prostate cancer, melanoma, sarcomas, carcinomas, Wilms tumor, endometrial cancer, glioblastoma, squamous cell cancer, astrocytomas, salivary gland carcinoma, vulvar cancer, penile carcinoma, and head-and-neck cancer; the cancer may be breast cancer, a brain tumor, prostate cancer, melanoma, or a metastatic cancer thereof.

Additionally or alternatively, in some embodiments of the methods disclosed herein, the complex is administered intravenously, intramuscularly, intraarterially, intrathecally, intracapsularly, intradermally, intraperitoneally, intralesionally, transtracheally, subcutaneously, intracerebroventricularly, orally, intranasally, rectally, topically, or via inhalation. In certain embodiments, the complex is administered into the cerebral spinal fluid or blood of the subject. In some embodiments of the methods disclosed herein, the radioactive levels emitted by the complex are detected between about 5 minutes to about 168 hours after the complex is administered. For example, the radioactive levels emitted by the complex may be detected at about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60, hours about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, about 90 hours, about 91 hours, about 92 hours, about 93 hours, about 94 hours, about 95 hours, about 96 hours, about 97 hours, about 98 hours, about 99 hours, about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, about 120 hours, about 121 hours, about 122 hours, about 123 hours, about 124 hours, about 125 hours, about 126 hours, about 127 hours, about 128 hours, about 129 hours, about 130 hours, about 131 hours, about 132 hours, about 133 hours, about 134 hours, about 135 hours, about 136 hours, about 137 hours, about 138 hours, about 139 hours, about 140 hours, about 141 hours, about 142 hours, about 143 hours, about 144 hours, about 145 hours, about 146 hours, about 147 hours, about 148 hours, about 149 hours, about 150 hours, about 151 hours, about 152 hours, about 153 hours, about 154 hours, about 155 hours, about 156 hours, about 157 hours, about 158 hours, about 159 hours, about 160 hours, about 161 hours, about 162 hours, about 163 hours, about 164 hours, about 165 hours, about 166 hours, about 167 hours, or about 168 hours (or any range including and/or in between any two of these values) after the complex is administered.

In certain embodiments of the methods disclosed herein, the radioactive levels emitted by the complex are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, or any range including and/or in between any two of these values.

The effectiveness of such a complex may be determined by computing the area under the curve (AUC) tumor: AUC normal tissue ratio. In some embodiments, the complex has an area under the curve (AUC) tumor: AUC normal tissue ratio of about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, or any range including and/or in between any two of these values.

In another aspect, the present disclosure provides a method for detecting acidic diseased tissue in a subject in need thereof comprising (a) administering to the subject an effective amount of a complex of any embodiment described herein, wherein the complex is configured to localize to an extracellular environment having a pH that is lower than 7.4; and (b) detecting the presence of acidic diseased tissue (that is, a tissue with an extracellular environment having a pH that is lower than 7.4) in the subject by detecting radioactive levels emitted by the complex that are higher than a reference value. In some embodiments, the subject is human. Examples of acidic diseased tissue include atherosclerotic plaques, ischemic myocardium, tissues impacted by stroke, and tumors. In some embodiments, the extracellular environment of the acidic diseased tissue has a pH value that is 7.3, 7.2, 7.1, 7.0, 6.9, 6.8, 6.7, 6.6, 6.5, 6.4, 6.3, 6.2, 6.1, 6.0, or lower. For example, the extracellular environment of the tissue may have a pH that is 7.1, 7.0, 6.9, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0, 1.8, 1.6, 1.4, 1.2, 1.0, or any range including and/or in between any two of these values. The radioactive levels emitted by the complex may be expressed as the percentage injected dose per gram tissue (% ID/g).

In some embodiments of the methods disclosed herein, the radioactive levels emitted by the complex are detected using positron emission tomography or single photon emission computed tomography.

Additionally or alternatively, in some embodiments of the methods disclosed herein, the complex is administered intravenously, intramuscularly, intraarterially, intrathecally, intracapsularly, intraorbitally, intradermally, intraperitoneally, intralesionally, subcutaneously, intracerebroventricularly, orally, rectally, topically, vaginally, or via inhalation. In some embodiments of the methods disclosed herein, the radioactive levels emitted by the complex are detected between about 5 minutes to about 168 hours after the complex is administered. For example, the radioactive levels emitted by the complex may be detected at about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60, hours about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, about 90 hours, about 91 hours, about 92 hours, about 93 hours, about 94 hours, about 95 hours, about 96 hours, about 97 hours, about 98 hours, about 99 hours, about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, about 120 hours, about 121 hours, about 122 hours, about 123 hours, about 124 hours, about 125 hours, about 126 hours, about 127 hours, about 128 hours, about 129 hours, about 130 hours, about 131 hours, about 132 hours, about 133 hours, about 134 hours, about 135 hours, about 136 hours, about 137 hours, about 138 hours, about 139 hours, about 140 hours, about 141 hours, about 142 hours, about 143 hours, about 144 hours, about 145 hours, about 146 hours, about 147 hours, about 148 hours, about 149 hours, about 150 hours, about 151 hours, about 152 hours, about 153 hours, about 154 hours, about 155 hours, about 156 hours, about 157 hours, about 158 hours, about 159 hours, about 160 hours, about 161 hours, about 162 hours, about 163 hours, about 164 hours, about 165 hours, about 166 hours, about 167 hours, or about 168 hours (or any range including and/or in between any two of these values) after the complex is administered.

### Kits

The present technology provides kits containing components suitable for diagnosing in a patient a tissue that includes an extracellular environment having a pH that is lower than 7.4, such as an atherosclerotic plaque, an ischemic myocardium, a tissue impacted by stroke, inflamed tissue, and/or a cancer tissue (such as a solid tumor). In one aspect, the kits include at least one compound or complex of the present technology disclosed herein and instructions for use. The compound or complex may be provided in the form of a prefilled syringe or autoinjection pen containing a sterile, liquid formulation or lyophilized preparation of the compound or complex (e.g., Kivitz et al., Clin. Ther. 28:1619-29 (2006)). In any embodiment herein, the instructions for use may include instructions for generating a complex of any embodiment described herein. In any embodiment herein, the instructions for use may include instructions for performing any embodiment of any method described herein.

If the kit components are not formulated for oral administration, a device capable of delivering the kit components through some other route may be included. Examples of such devices include syringes (for parenteral administration) or inhalation devices. The kit components may be packaged together or separated into two or more containers. In some embodiments, the containers may be vials that contain sterile, lyophilized formulations of a compound or complex of the present technology that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

### Examples

Materials and Methods

Representative General Information. SEQ ID NO. 1 was prepared via fluorenylmethyloxycarbonyl (fmoc) solid-phase peptide synthesis, whereafter SEQ ID NO. 1 was conjugated with (N-(3,11,14,22,25,33-hexaoxo-4,10,15,21,26,32-hexaaza-10,21,32-trihydroxytetratriacontane)maleimide. The resulting compound was (D-Ala)-(D-Cys[X³])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D- Asp)-(D-Thr)-(D-Leu)-(D-Leu)-(D-Leu)-(D-Asp)-(D-Leu)-(D-Leu)-(D-Trp) (SEQ ID NO. 25) where X³ is attached to the sulfur atom of the indicated D-cysteine residue and has the following structural formula:

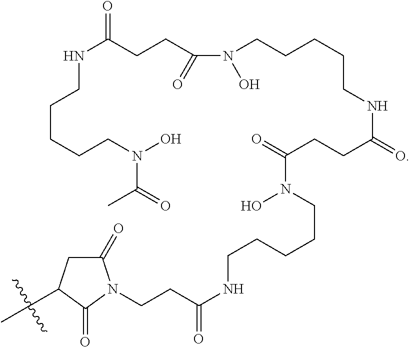 [SMILES: *C1CC(=O)N(CCC(=O)NCCCCCN(O)C(=O)CCC(=O)NCCCCCN(O)C(=O)CCC(=O)NCCCCCN(O)C(C)=O)C1=O]

The resulting compound is hereafter referred to in the Examples as “DFO-cys-Var3pHLIP.”

Similarly, SEQ ID NO. 239 was prepared via fluorenylmethyloxycarbonyl (fmoc) solid-phase peptide synthesis, whereafter SEQ ID NO. 239 was conjugated with “DFO-SqOEt”, “p-SCN-Bn-3,4,3-Li(1,2-hopo)”, “p-SCN-Bn-DFO*”, or “p-SCN-Bn-DFO” (structural formulas provided below) to respectively provide “DFOsqa-lys-Var3pHLIP,” “HOPO-lys-Var3pHLIP,” “DFO*-lys-Var3pHLIP,” and “DFO-lys-Var3pHLIP.”

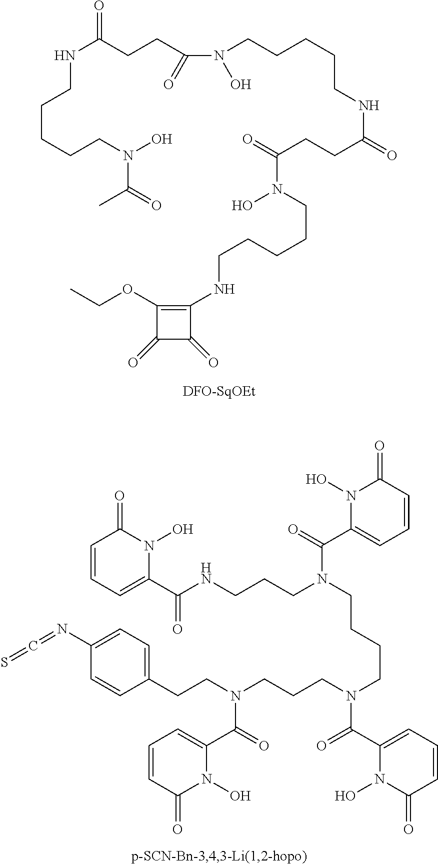 [SMILES: CCOc1c(NCCCCCN(O)C(=O)CCC(=O)NCCCCCN(O)C(=O)CCC(=O)NCCCCCN(O)C(C)=O)c(=O)c1=O ; O=C(NCCCN(CCCCN(CCCN(CCc1ccc(N=C=S)cc1)C(=O)c1cccc(=O)n1O)C(=O)c1cccc(=O)n1O)C(=O)c1cccc(=O)n1O)c1cccc(=O)n1O]

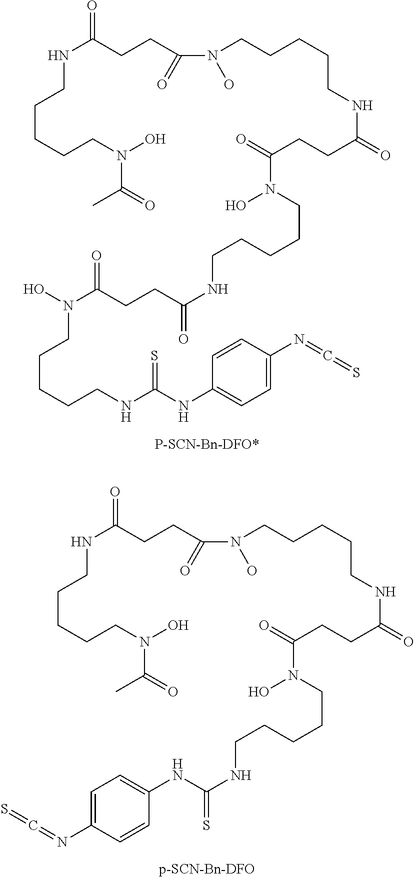 [SMILES: CC(=O)N(O)CCCCCNC(=O)CCC(=O)N(O)CCCCCNC(=O)CCC(=O)N(O)CCCCCNC(=O)CCC(=O)N(O)CCCCCNC(=S)Nc1ccc(N=C=S)cc1 ; CC(=O)N(O)CCCCCNC(=O)CCC(=O)N(O)CCCCCNC(=O)CCC(=O)N(O)CCCCCNC(=S)Nc1ccc(N=C=S)cc1]

DFOsqa-lys-Var3pHLIP, HOPO-lys-Var3pHLIP, DFO*-lys-Var3pHLIP, and DFO-lys-Var3pHLIP are each Ac-(D-Ala)-(D-Lys[X⁴])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)- (D-Leu)-(D-Asp)-(D-Leu)-(D-Leu)-(D-Trp)-(D-Ala) (SEQ ID NO. 242), where X⁴ is attached to the F-nitrogen atom of the indicated D-lysine residue and X⁴ differs for each of DFOsqa-lys-Var3pHLIP, HOPO-lys-Var3pHLIP, DFO*-lys-Var3pHLIP, and DFO-lys-Var3pHLIP.

In particular, X⁴ for DFOsqa-lys-Var3pHLIP has the following structural formula:

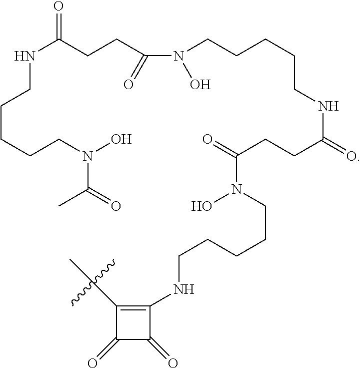 [SMILES: *c1c(NCCCCCN(O)C(=O)CCC(=O)NCCCCCN(O)C(=O)CCC(=O)NCCCCCN(O)C(C)=O)c(=O)c1=O]

X⁴ for HOPO-lys-Var3pHLIP has the following structural formula:

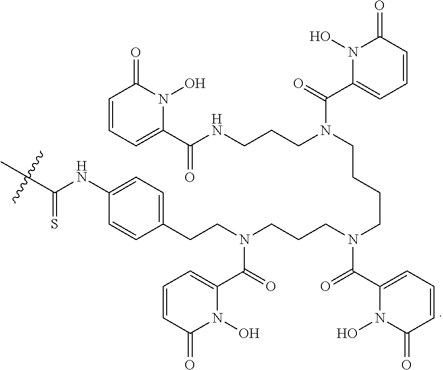 [SMILES: *C(=S)Nc1ccc(CCN(CCCN(CCCCN(CCCNC(=O)c2cccc(=O)n2O)C(=O)c2cccc(=O)n2O)C(=O)c2cccc(=O)n2O)C(=O)c2cccc(=O)n2O)cc1]

X⁴ for DFO*-lys-Var3pHLIP has the following structural formula:

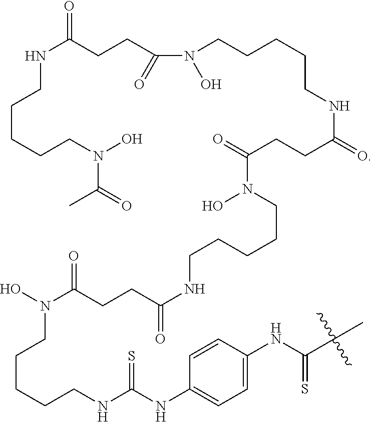 [SMILES: *C(=S)Nc1ccc(NC(=S)NCCCCCN(O)C(=O)CCC(=O)NCCCCCN(O)C(=O)CCC(=O)NCCCCCN(O)C(=O)CCC(=O)NCCCCCN(O)C(C)=O)cc1]

X⁴ for DFO-lys-Var3pHLIP has the following structural formula:

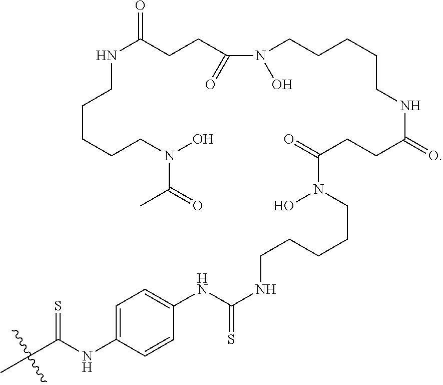 [SMILES: *C(=S)Nc1ccc(NC(=S)NCCCCCN(O)C(=O)CCC(=O)NCCCCCN(O)C(=O)CCC(=O)NCCCCCN(O)C(C)=O)cc1]

In addition, Ac-(D-Ala)-(D-Lys)-(D-Glu)-(D-Gln)-(D-Asn)-(D-Pro)-(D-Ile)-(D-Tyr)-(D-Trp)-(D-Ala)-(D-Arg)-(D-Tyr)-(D-Ala)-(D-Asp)-(D-Trp)-(D-Leu)-(D-Phe)-(D-Thr)-(D-Thr)-(D-Pro)-(D-Leu)-(D-Leu)-(D-Leu)-(D-Leu)-(D-Asp)-(D-Leu)- (D-Ala)-(D-Leu)-(D-Leu)-(D-Val)-(D-Asp)-(D-Ala)-(D-Asp)-(D-Glu)-Gly-(D-Thr)-Gly (SEQ. ID NO. 243; “Lys-WT2D”) (see “Lys-WT2” in Table 4 of the present disclosure) was prepared via fluorenylmethyloxycarbonyl (fmoc) solid-phase peptide synthesis, whereafter SEQ ID NO. 243 was conjugated with p-SCN-Bn-DFO to provide “DFO-lys-WT2DpHLIP.” DFO-lys-WT2DpHLIP is Ac-(D-Ala)-(D-Lys[X⁵])-(D-Glu)-(D-Gln)-(D-Asn)-(D-Pro)-(D-Ile)-(D-Tyr)-(D-Trp)-(D-Ala)-(D-Arg)-(D-Tyr)-(D-Ala)-(D-Asp)-(D-Trp)-(D-Leu)-(D-Phe)-(D-Thr)-(D-Thr)-(D-Pro)-(D-Leu)-(D-Leu)-(D-Leu)-(D-Leu)-(D- Asp)-(D-Leu)-(D-Ala)-(D-Leu)-(D-Leu)-(D-Val)-(D-Asp)-(D-Ala)-(D-Asp)-(D-Glu)-Gly-(D-Thr)-Gly (SEQ. ID NO. 244) where X⁵ is attached to the F-nitrogen atom of the indicated D-lysine residue and where X⁵ has the following structural formula:

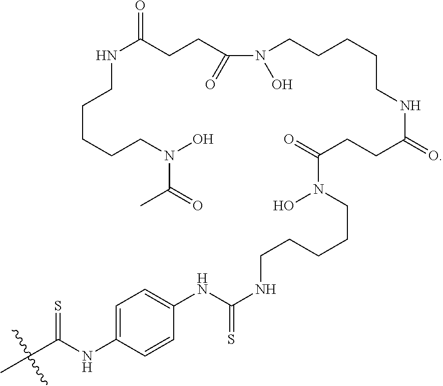 [SMILES: *C(=S)Nc1ccc(NC(=S)NCCCCCN(O)C(=O)CCC(=O)NCCCCCN(O)C(=O)CCC(=O)NCCCCCN(O)C(C)=O)cc1]

Exemplary Synthesis of DFO-SqOEt: DFO-SqOEt was synthesized according to protocols descrbed in S. E. Rudd, P. Roselt, C. Cullinane, R. J. Hicks, P. S. Donnelly, Chemical Communications 2016, 52, 11889-11892 (DOI: 10.1039/C6CC05961A). In particular, a mixture of deferoxamine mesylate (0.20 g, 0.31 mmol, from Sigma-Aldrich) and N,N-diisopropylethylamine (0.05 mL, 0.3 mmol) was stirred in ethanol (6 mL) at 50° C. After 1 h, 3,4-diethoxy-3-cyclobutene-1,2-dione (0.1 mL, 0.7 mmol) in ethanol (9 mL) was added. After a further 30 mins of stirring at 50° C. the solvent was removed under reduced pressure, and the residue triturated with ethanol (3×10 mL). The product was dried in vacuo to give DFO-SqOEt as a white powder (0.17 g, 83%). ¹H NMR (d₆-DMSO, 500 MHz) δ 9.61 (s, 6H), 8.77 (t, J=5.8 Hz, 1H), 8.58 (t, J=5.7 Hz, 1H), 7.77 (d, J=4.7 Hz, 5H), 4.64 (p, J=6.9 Hz, 5H), 3.45 (t, J=7.0 Hz, 3H), 3.38 (s, 5H), 3.26 (dd, J=13.0, 6.6 Hz, 1H), 3.00 (dd, J=12.7, 6.4 Hz, 10H), 2.56 (d, J=6.5 Hz, 1H), 2.26 (t, J=7.2 Hz, 1H), 1.96 (s, 7H), 1.50 (d, J=6.6 Hz, 3H), 1.42-1.31 (m, 3H), 1.24 (ddd, J=20.2, 14.7, 8.0 Hz, 2H); ¹³C NMR (d₆-DMSO, 101 MHz) δ 189.4, 189.3, 182.0, 181.8, 176.9, 176.5, 172.6, 172.2, 172.00, 171.3, 170.1, 70.2, 68.8, 68.7, 47.1, 47.0, 46.8, 43.7, 43.4, 39.5, 38.4, 30.1, 29.9, 29.6, 28.8, 27.6, 26.0, 25.8, 23.5, 22.9, 20.3, 15.6; ESI MS [M*H⁺]: 685.3768, calculated for (C₃₁H₅₃N₆O₁₁)+: 685.3767, [M+Na⁺]: 707.3589, calculated for (C₃₁H₅₂NaN₆O₁₁)⁺: 707.3586.

Exemplary Conjugation of DFO-SqOEt to SEQ ID NO. 239 to yield DFOsqa-lys-Var3pHLIP: 10 mg of SEQ ID NO. 239 (>95% purity) was reacted with 5 mg DFO-SqOEt in 100 μl DIEA and 1.2 ml DMF for 72 to 96 hours to yield DFOsqa-lys-Var3pHLIP. The identity of the peptide from conjugation was checked by Mass Spectrometry (MS), and the purity analyzed by analytical C18 reverse phase high performance liquid chromatography (RP-HPLC). The peptide was purified through RP-HPLC column(s) utilizing varying buffer systems. The buffer systems include an aqueous solution paired with an organic phase modifier consisting of 0.1% TFA in water (Buffer A) and 100% acetonitrile. After completion of purification procedure, the desired DFOsqa-lys-Var3pHLIP fractions were collected and lyophilized via jar lyophilization method to provide 5 mg of DFOsqa-lys-Var3pHLIP at a purity of >95%.

Exemplary Synthesis of p-SCN-Bn-3,4,3-Li(1,2-hopo): p-SCN-Bn-3,4,3-Li(1,2-hopo) was synthesized according to protocols descrbed in N. Bhupathiraju, A. Younes, M. Cao, J. Ali, H. T. Cicek, K. M. Tully, S. Ponnala, J. W. Babich, M. A. Deri, J. S. Lewis, L. C. Francesconi, C. M. Drain, Org Biomol Chem 2019, 17, 6866-6871 (DOI: 10.1039/c9ob01068h).

Exemplary Conjugation of p-SCN-Bn-3,4,3-Li(1,2-hopo) to SEQ ID NO. 239 to yield HOPO-lys-Var3pHLIP: 8 mg of SEQ ID NO. 239 (>95% purity) was reacted with 5 mg p-SCN-Bn-3,4,3-Li(1,2-hopo) in 80 μl DIEA and 1.2 ml DMSO for 3 to 4 hours to yield HOPO-lys-Var3pHLIP. The identity of the peptide from conjugation was checked by Mass Spectrometry (MS), and the purity analyzed by analytical C18 reverse phase high performance liquid chromatography (RP-HPLC). The peptide was purified through RP-HPLC column(s) utilizing varying buffer systems. The buffer systems include an aqueous solution paired with an organic phase modifier consisting of 0.1% TFA in water (Buffer A) and 100% acetonitrile. After completion of purification procedure, the desired HOPO-lys-Var3pHLIP fractions were collected and lyophilized via jar lyophilization method to provide 6 mg of HOPO-lys-Var3pHLIP at a purity of >95%.

Exemplarly Labeling ([⁸⁹Zr]Zr-DFO-cys-Var3pHLIP): About 400 μg of DFO-cys-Var3pHLIP was dissolved in anhydrous dimethyl sulfoxide to yield a clear 2.0 mg/mL solution (solution A). Separately, [⁸⁹Zr]Zr-oxalate solution in 1M oxalic acid was diluted with 1M oxalic acid to a total volume of 100 mL; the pH was then adjusted to about 7.4 using 1M aqueuous sodium carbonate to yield solution B. About 40 μL of solution A was added to solution B; the resulting solution was allowed to react at 50° C. for 30 minutes in a mixing block. An Oasis HLB Plus Light or C18 Sep-Pak Light cartridge (Waters, Milford, MA) was used to remove unbound activity. The pure radiolabeled complex [⁸⁹Zr]Zr-DFO-cys-Var3pHLIP was eluted with EtOH and diluted with sterile phosphate buffered saline (PBS) with purities >93% before administration to animals. [⁸⁹Zr]Zr-DFOsqa-lys-Var3pHLIP, [⁸⁹Zr]Zr-HOPO-lys-Var3pHLIP, [⁸⁹Zr]Zr-DFO*-lys-Var3pHLIP, [⁸⁹Zr]Zr-DFO-lys-Var3pHLIP, and [⁸⁹Zr]Zr-DFO-lys-WT2DpHLIP were prepared according to similar protocols.

In vivo animal PET imaging and biodistribution. All injections were less than 200 μL with <10% EtOH in sterile PBS. PET images were obtained with the mice under anesthesia in an Inveon PET-CT at at 4-144 h p.i. All images were analyzed using 3D Slicer (www.slicer.org). Dissections for ex vivo biodistribution were performed on mice after C02 asphyxiation or cervical dislocation while anesthetized at reported time points. Weight of the syringe prior to injection and after injection was used to determine the mass of injectate. Activity of the syringe prior to injection and after injection was used to determine the percent of injectate administered. The mass injected was corrected by the percent of radioactivity injected. Four to five aliquots (10 μL) were weighed and counted as internal standards for each study. All of the collected organs were counted using an automatic gamma counter (Wizard 3″, PerkinElmer, Waltham, MA). The total injected dose was found as the mass injected dose×fraction radioactivity injected×internal standard average counts/g. The percent injected dose (% ID) was determined as the counts for the tissue×100/total injected dose. The % ID/g was calculated as the % ID/tissue weight. The average and standard deviation of the % ID and % ID/g was determined using normal methods (n-1) for each set of mice.

In Vitro and In Vivo Studies with the Complexes of the Present Technology

These Examples demonstrate that the complexes of the present technology are useful in methods for detecting acidic diseased tissues (e.g., tumors) in a subject.

4T1 orthotopic BALB c mouse model. Animal studies were conducted according to MSKCC IACUC-approved animal protocol.

The 4T1 cells derived from spontaneous breast tumor in a BALB/c mouse (Karmanos Cancer Institute, Detroit, MI) were cultured in Dulbecco's modified Eagle's high glucose media with 10% FCS, 2 mM L-glutamine, penicillin, and streptomycin. The 4T1 cells derived from ATCC (Manassas, VA) were cultured in RPMI-1640 medium modified to contain 2 mM L-glutamine, 10 mM HEPES, 1 mM sodium pyruvate, 4.5 g/L glucose, 1.5 g/L NaHCO₃, penicillin, and streptomycin.

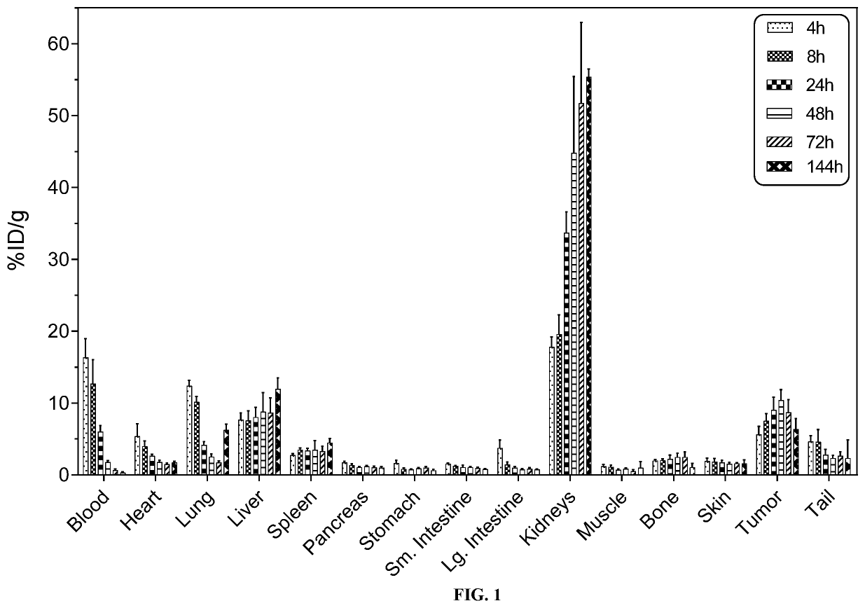

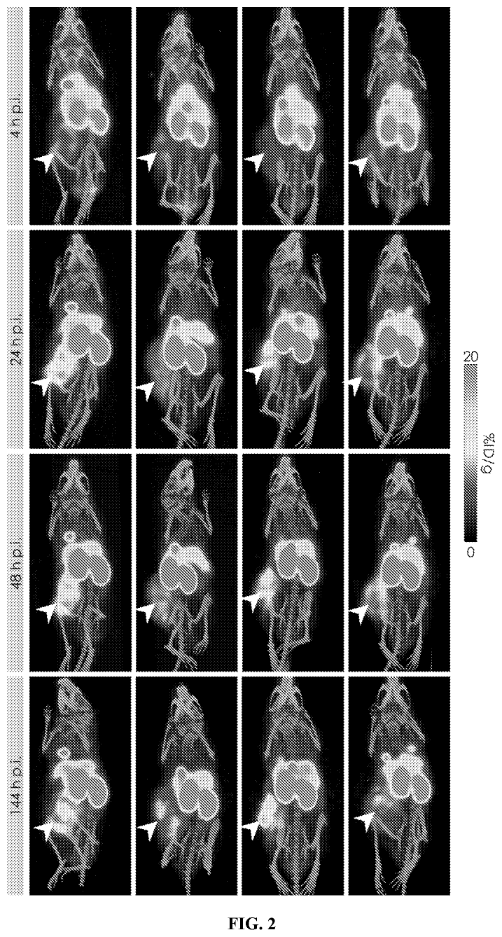

The 4T1 cells described above were removed from the flasks containing the culture, concentrated, and resuspended in minimal media for cell counting. The cells were then diluted to contain approximately 1 million cells in 30 μL of media (for orthotopic allografts). Following an MSKCC Institutional Animal Care and Use Committee approved protocol, 5-7 week old BALB/c mice (Charles River Laboratories, Wilmington, MA) were surgically implanted with about 100,000 4T1 cancer cells into the lower mammary fat pad of the right side of the animal using aseptic surgical techniques and sterile staple closures while the mice were under anesthetic. Additionally, mice were given injections of meloxicam (24 h pain killer) in the scruff and bupivacaine intradermally prior to surgical incisions. One day post surgery, the mice were again given meloxicam and checked to ensure that the closure was healing. Two and three days post surgery, the mice were checked to ensure that the animals were healthy and recovering well from the surgery. Five to seven days post surgery, the staples were removed. Three weeks post surgery when tumor volume had reached about 0.5 cm³ to about 1 cm³, the mice were injected with 10-100 μCi (about 5 μg) of [⁸⁹Zr]Zr-DFO-cys-Var3pHLIP. Four mice per time point were utilized for both ex vivo biodistribution and in vivo imaging. For ex vivo biodistribution analysis, mice sacrificed at 4 hours post-injection, 8 hours post-injection, 24 hours post-injection, 48 hours post-injection, 72 hours post-injection, and 144 hours post-injection (FIG. 1). For in vivo imaging, PET scans were performed on mice at 4 hours post-injection, 24 hours post-injection, 48 hours post-injection, and 144 hours post-injection, where representative images are provided in FIG. 2.

As illustrated in FIG. 1, [⁸⁹Zr]Zr-DFO-cys-Var3pHLIP exhibits long circulatory half-life providing an extended period for accumulation in tumor tissue. Tumor uptake continues to increase until approximately 48 h post-administration. With the exception of the liver and kidneys, uptake in other tissues is either minimal (e.g., muscle, gastrointestinal tract) or rapidly clears following administration (e.g., cardiac and respiratory tissues). For the liver and kidneys, the uptake does not pose a significant concern for use of [⁸⁹Zr]Zr-DFO-cys-Var3pHLIP as a diagnostic. Further, given the significantly higher relative uptake and retention in tumor tissue illustrated in these studies, should there be concerns with systemic administration such concerns are alleviated via intratumoral administration of complexes of the present technology (see, e.g., “U373 human glioblastoma mouse model” below in this Examples section).

The in vivo imaging data (see FIG. 2) corroborates the ex vivo biodistribution data and further evidences high uptake in 4T1 tumors, with focal tumor uptake as high as about 15% ID/g.

U373 human glioblastoma mouse model. Animal studies were conducted according to MSKCC IACUC-approved animal protocol. U373 cells were provided according to procedures as described in Chow, K. K, et al. “T cells redirected to EphA2 for the immunotherapy of glioblastoma” Mol Ther, 2013, 21(3), 629-37. Following an MSKCC Institutional Animal Care and Use Committee approved protocol, the brains of 3-6 week old ICR-SCID mice (IcrTac:ICR-Prkdcscid; Taconic, Hudson, NY) were inoculated via stereotaxic surgery with about 10⁵ U373.eGFP.ffLuc cells in 2 μL of media; the cells were implanted 3 mm deep to the bregma, corresponding to the center of the right caudate nucleus, over 5 minutes. When tumor volume had reached about 0.1 cm³, about 10 μCi (about 5 μg) of [⁸⁹Zr]Zr-DFO-cys-Var3pHLIP was administed intratumorally via stereotaxic guidance.

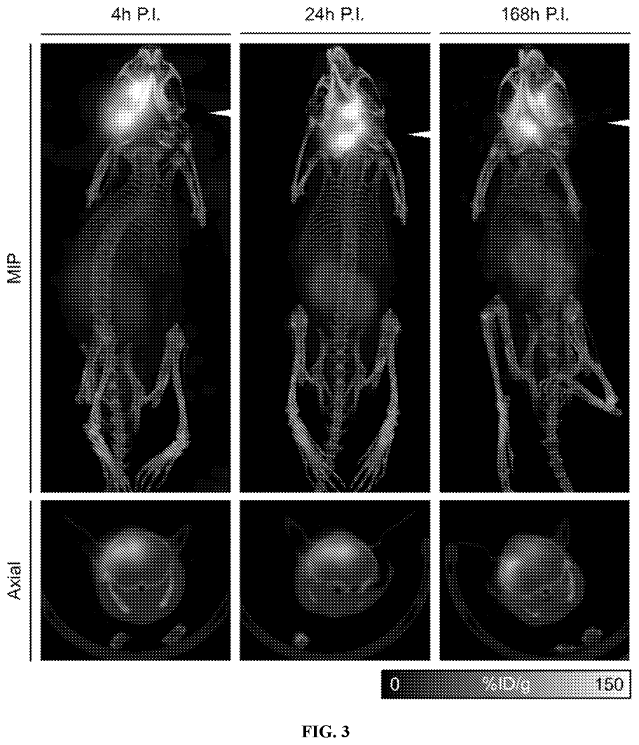

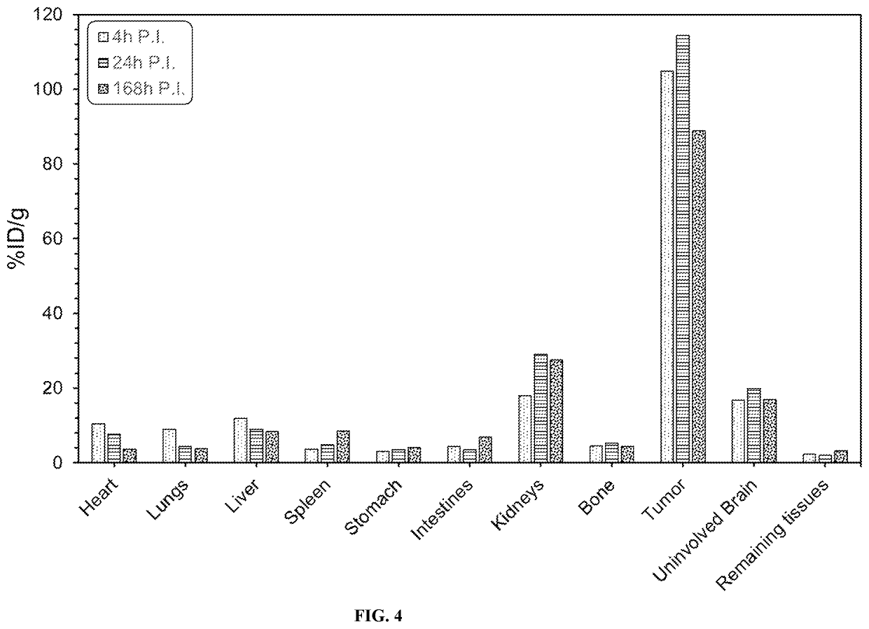

In vivo PET images were performed on the mice at 4 hours post-injection, 24 hours post-injection, and 168 hours post-injection, where representative maximum intensity projection (“MIP”) and coronal slice (“Axial”) PET/CT images are provided in FIG. 3; mean organ uptake levels were determined by volume-of-interest analysis of the in vivo PET image data where the results are provided in FIG. 4.

As illustrated by FIGS. 3-4, [⁸⁹Zr]Zr-DFO-cys-Var3pHLIP was retained at high levels (>80% ID/g mean; >150% ID/g maximum) at times exceeding one week post-injection. The multi-focal uptake pattern suggests that [⁸⁹Zr]Zr-DFO-cys-Var3pHLIP injected intratumorally at the primary tumor site may enable delineation of infiltrating malignant tissue.

These results demonstrate that the complexes of the present technology are useful in methods for detecting solid tumors in a subject. Accordingly, the compounds and complexes disclosed herein are useful in methods for detecting acidic diseased tissues in a subject.

### Use of the Complexes of the Present Technology to Detect Acidic Diseased Tissues in a Subject

This Example demonstrates that the complexes of the present technology are useful in methods for detecting acidic diseased tissues in a subject.

Cerebral ischemia will be induced by occlusion of the right middle cerebral artery for 30 min. Wild-type (WT) mice will be given a complex of the present technology (500-600 μCi (9-11 nmol) for in vivo imaging) at 0, 6, 24 and 48 h after ischemia. Mice will be sacrificed at no later than 48 hours after receiving the complex. PET/CT (e.g., at 2 p.i. and 4 h p.i.) and SPECT/CT (e.g., at 6 p.i. and 24 h p.i.) imaging studies will be carried out.

It is anticipated that the complexes disclosed herein will localize to the acidic diseased brain tissue (tissue impacted by ischemia), with minimal accumulation in non-target tissues.

These results demonstrate that the complexes of the present technology are useful in methods for detecting acidic diseased tissues in a subject.

### Biophysical Studies

Biophysical studies were performed on “cold” versions of [⁸⁹Zr]Zr-DFO-cys-Var3pHLIP, [⁸⁹Zr]Zr-DFOsqa-lys-Var3pHLIP, [⁸⁹Zr]Zr-HOPO-lys-Var3pHLIP, [⁸⁹Zr]Zr-DFO*-lys-Var3pHLIP, [⁸⁹Zr]Zr-DFO-lys-Var3pHLIP, and [⁸⁹Zr]Zr-DFO-lys-WT2DpHLIP by substituting ⁸⁹Zr radionuclide with the naturally occurring Zr, where the “cold” version is respectively hereafter referred to as “Zr-DFO-cys-Var3pHLIP,” “Zr-DFOsqa-lys-Var3pHLIP,” “Zr-HOPO-lys-Var3pHLIP,” “Zr-DFO*-lys-Var3pHLIP,” “Zr-DFO-lys-Var3pHLIP,” and “Zr-DFO-lys-WT2DpHLIP.” Collectively, these “cold” versions are referred to as “Cold-Zr-pHLIP.”

ExemplarLy synthesis and characterization of Zr-DFO-cys-Var3pHLIP: A 10 mg/mL solution of zirconium chloride was prepared by dissolving 5.0 mg zirconium chloride (Sigma-Aldrich #357405) in 500 μL of 18.2 MΩ-cm water. 60 μL of this solution was transferred to a centrifuge vial (solution A). To solution A, 100 μL of 0.1 M oxalic acid was added, and the pH was then adjusted to 7.4 by sequential additions of 0.1 M sodium carbonate solution. 2.0 mg of DFO-cys-Var3pHLIP was added to a separate vial and dissolved by addition of 50 μL dimethyl sulfoxide (solution B). Solutions A and B were combined, and allowed to react for 1 hour on a thermomixer at 40° C. with moderate shaking (500 rpm). Subsequently, the Zr-DFO-cys-Var3pHLIP product was desalted via solid phase extraction (C18 Sep-Pak Plus; Waters) by first diluting the crude reaction mixture in 3 mL of water and loading onto the extraction cartridge, then ‘washing’ (i.e., eluting) with 10 mL water, and finally eluting the Zr-DFO-cys-Var3pHLIP with 2 mL of acetonitrile into a 50 mL centrifuge tube. 2 mL of water was added to the acetonitrile eluate, and the purified solid Zr-DFO-cys-Var3pHLIP was obtained by lyophilization. Zr-DFO-cys-Var3pHLIP was characterized by UPLC-MS (C18 column, 5-95% acetonitrile/water gradient over 6 minutes). A single UV peak was observed at 214 nm at 4.00 minutes, with base peak m/z=1341.2 ([Zr-DFO-cys-Var3pHLIP+3H]³⁺); expected: 1342.6. A secondary peak (˜10% relative abundance) was observed at m/z=1006.3 ([Zr-DFO-cys-Var3pHLIP+4H]⁴⁺); expected: 1007.1. These results confirm the identity and purity of Zr-DFO-cys-Var3pHLIP. Similar protocols were utilized to provide each of Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP.

Exemplary preparation of Zr-DFO-cys-Var3pHLIP used in biophysical studies: Zr-DFO-cys-Var3pHLIP was dissolved in 10 mM phosphate buffer (pH 7.6) and concentration was calculated by absorbance at 280 nm using the extinction coefficient F280=12960 M⁻¹cm⁻¹. This was similarly performed for each of Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP.

Liposome preparation: Small unilamellar vesicles were used as model membranes and were prepared by extrusion. POPC (1-palmitoyl-2-oleoyl-sn-glycero-3-phosphocholine) lipids were dissolved in chloroform, then desolvated by rotary evaporation for two hours under vacuum. The resulting POPC film was rehydrated in 10 mM phosphate buffer at pH 8, vortexed, and extruded twenty-one times through a membrane with a pore size of 50 nm.

Steady-state fluorescence measurements: Steady-state fluorescence spectra were measured using a PC1 spectrofluorometer (ISS). The tryptophan fluorescence was excited using an excitation wavelength of 295 nm. Excitation and emission slits were set to 8 nm, and excitation and emission polarizers were set to 54.7° and 0.0°, respectively. A buffer-with-POPC sample were used as baselines and were subtracted from signals measured in presence of POPC.

pH dependence measurements: Measurements of pH dependence were taken with the PC1 spectrofluorometer by using the shift in the position of maximum of peptide fluorescence as an indication of changes of the Cold-Zr-pHLIP environment at varying pH for each of Zr-DFO-cys-Var3pHLIP, Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP. After the addition of hydrochloric acid, the pH of each solution containing 4.65 μM of a particular Cold-Zr-pHLIP and 0.93 mM POPC were measured using an Orion PerHecT ROSS Combination pH Micro Electrode and an Orion Dual Star pH and ISE Benchtop Meter (Thermo Fisher Scientific) before and after spectrum measurement to ensure equilibration. The tryptophan fluorescence spectrum at each pH was recorded, and the spectra were analyzed using the Protein Fluorescence and Structural Toolkit (PFAST) (5) to determine the positions of spectral maxima (λₘₐₓ). The position of λₘₐₓ was plotted as a function of pH, such that λₘₐₓ^{initial}—position of spectral maximum at high pH and λₘₐₓ^{final}—position of spectral maximum at low pH. The pH-dependence was fit with the Henderson-Hasselbach equation (see Eq. 1 below) using OriginLab software to determine the cooperativity (n) and transition mid-point (pK) of transition.

$$Normalized pH dependence = {λ}_{max}^{final} + \frac{( {λ}_{max}^{initial} - {λ}_{max}^{final} )}{1 + {10}^{n ( pH - pK )}} \qquad Eq . 1$$

Steady-state circular dichroism measurements: Steady-state CD was measured using an MOS-450 spectrometer (Bio-Logic Science Instruments) in the range of 190 to 260 nm with a step size of 1 nm, and with temperature control set to 25.0° C. A buffer-with-POPC sample were used as baselines and were subtracted from signals measured in presence of POPC.

Kinetics measurements: To follow Zr-pHLIP insertion of each of Zr-DFO-cys-Var3pHLIP, Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP into the lipid bilayer of POPC liposomes in real-time, kinetics measurements were performed using a SFM-300 mixing system (Bio-Logic Science Instruments) in combination with a MOS-450 spectrometer with its temperature control set to 25.0° C. For each of Zr-DFO-cys-Var3pHLIP, Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP, Cold-Zr-pHLIP pre-mixed with POPC liposomes in phosphate buffer at pH 8 were mixed (5 ms dead time) with acid to drop the pH from pH 8 to pH 5 and promote Cold-Zr-pHLIP insertion into the bilayers. The insertion process was monitored by recording changes of the tryptophan fluorescence of each Cold-Zr-pHLIP excited at 295 nm and using a cut off filter at 320 nm.

Data Analysis: All data were fit to the appropriate equations by nonlinear least squares curve fitting procedures employing the Levenberg Marquardt algorithm using Origin 8.5.

Results of Biophysical Studies: The biophysical measurements were performed to investigate Zr-DFO-cys-Var3pHLIP, Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP interactions with the lipid bilayer of liposomal membrane and identify the major parameters for insertion, such as a midpoint of the transition and rate of the insertion. The interaction of Zr-DFO-cys-Var3pHLIP, Zr-DFOsqa-lys-Var3pHLIP, Zr-HOPO-lys-Var3pHLIP, Zr-DFO*-lys-Var3pHLIP, Zr-DFO-lys-Var3pHLIP, and Zr-DFO-lys-WT2DpHLIP with membrane lipid bilayers was studied using POPC liposomes. Table 8 below provides the position of maximum (λₘₐₓ) of tryptophan fluorescence spectra of each of Cold-Zr-pHLIP measured in three states: State I (in buffer at pH 8), State II (in buffer at pH 8 in presence of POPC (1-palmitoyl-2-oleoyl-sn-glycero-3-phosphocholine) liposomes) and State III (in buffer at pH 4 in presence of POPC liposomes).

TABLE 8

| | State I | State II | State III |
|---|---|---|---|
| | λₘₐₓ (nm) | λₘₐₓ (nm) | λₘₐₓ (nm) |
| Zr-DFO-cys-Var3pHLIP | 352.4 | 348.8 | 340.1 |
| Zr-DFO-lys-Var3pHLIP | 352.2 | 349.7 | 341.6 |
| Zr-DFO-lys-WT2DpHLIP | 347.2 | 346.8 | 338.9 |
| Zr-HOPO-lys-Var3pHLIP | 352.6 | 346.1 | 347.2 |
| Zr-DFO*-lys-Var3pHLIP | 352.0 | 348.2 | 341.4 |
| Zr-DFOsqa-lys-Var3pHLIP | 352.4 | 350.3 | 340.2 |

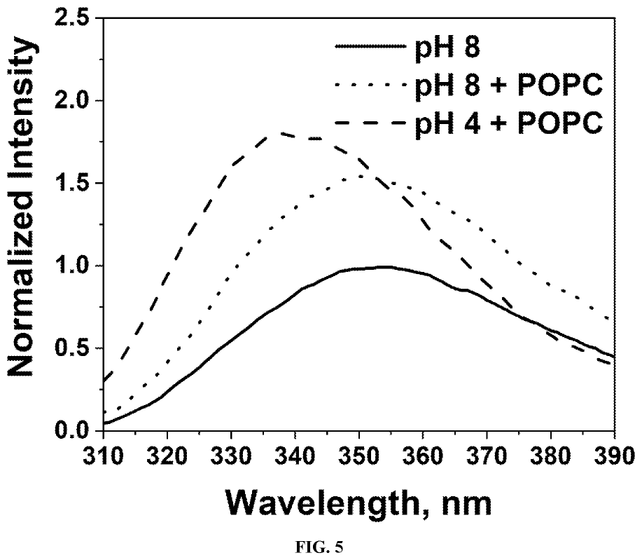

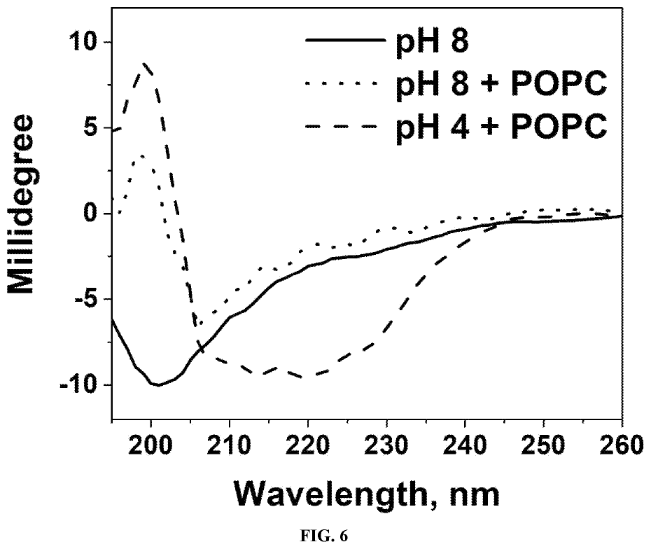

The data show Zr-DFO-cys-Var3pHLIP, Zr-DFOsqa-lys-Var3pHLIP, Zr-DFO*-lys-Var3pHLIP, Zr-DFO-lys-Var3pHLIP, and Zr-DFO-lys-WT2DpHLIP each exhibit pHLIP-like pH-dependent interactions with cell membranes monitored by changes of the peptide fluorescence emission of tryptophan residues (FIG. 5; Table 8 above) and circular dichroism (FIG. 6 for Zr-DFO-cys-Var3pHLIP; others not shown). Partition of each of Zr-DFO-cys-Var3pHLIP, Zr-DFOsqa-lys-Var3pHLIP, Zr-DFO*-lys-Var3pHLIP, Zr-DFO-lys-Var3pHLIP, and Zr-DFO-lys-WT2DpHLIP into lipid bilayer of the membrane at low pH is observed by increase of fluorescence signal and shift of the emission for 12 nm to short-wavelengths (FIG. 5; Table 8 above), and formation of alpha helical structure at low pH (FIG. 6), in accordance with peptides of the pHLIP family. See Reshetnyak Y K, Moshnikova A, Andreev O A, Engelman D M. “Targeting Acidic Diseased Tissues by pH-Triggered Membrane-Associated Peptide Folding.” Front Bioeng Biotechnol. 2020; 8:335 (Epub 2020/05/16; doi: 10.3389/fbioe.2020.00335; PubMed PMID: 32411684; PMCID: PMC7198868); Reshetnyak Y K, Segala M, Andreev O A, Engelman D M. “A monomeric membrane peptide that lives in three worlds: in solution, attached to, and inserted across lipid bilayers.” Biophys J. 2007; 93(7):2363-72 (Epub 2007/06/15; doi: 10.1529/biophysj.107.109967; PubMed PMID: 17557792; PMCID: PMC1965453); and Shen C, Menon R, Das D, Bansal N, Nahar N, Guduru N, Jaegle S, Peckham J, Reshetnyak Y K. “The protein fluorescence and structural toolkit: Database and programs for the analysis of protein fluorescence and structural data.” Proteins. 2008; 71(4):1744-54 (doi: 10.1002/prot.21857; PubMed PMID: 18175321). Zr-DFO-lys-WT2DpHLIP demonstrates some aggregation in solution, since position of maximum of fluorescence in State I is shifted to short wavelengths (347.2 nm; Table 8 above). Notably, Zr-HOPO-lys-Var3pHLIP does not exhibit insertion into the lipid bilayer of membrane of these studies at low pH, since there is no shift to short wavelengths in State III (347.2 nm vs 338-341 nm for other Cold-Zr-pHLIP; Table 8 above).

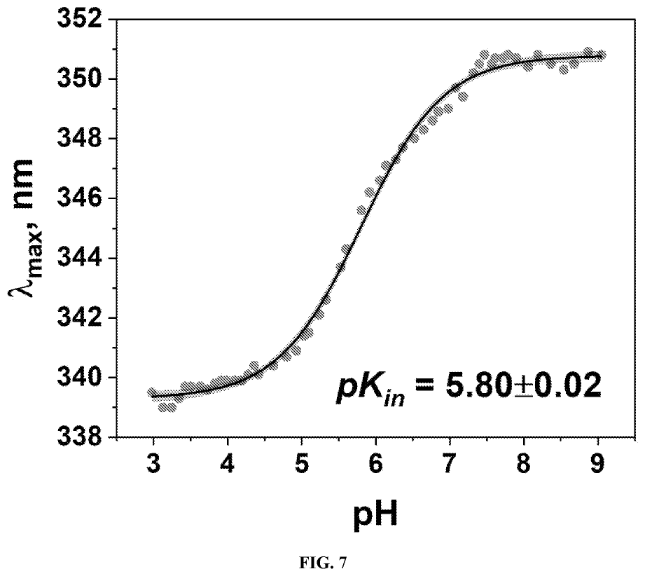

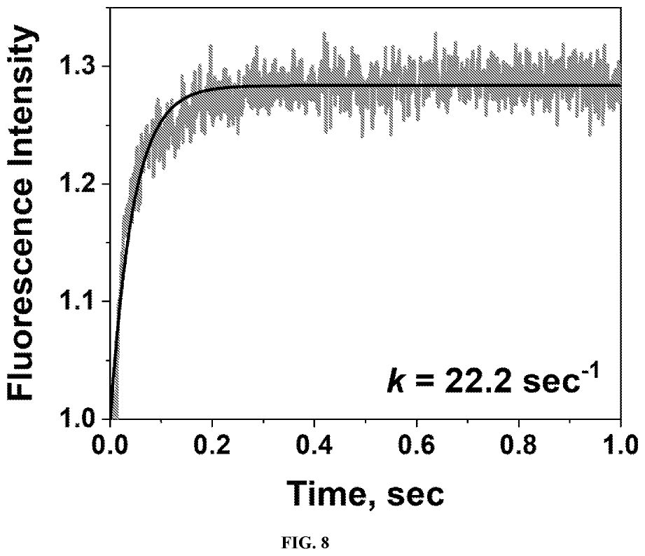

The midpoint of the transition for Zr-DFO-cys-Var3pHLIP insertion in liposomes is at about pH 5.8 (and cooperativity of transition is 0.8) as calculated from graph presented in FIG. 7. Thus, at pH 6.0, pH which is found at the surface of cancer cells within tumors, about 41% of Zr-DFO-cys-Var3pHLIP molecules are expected to be inserted across plasma membrane at equilibrium, while at pH 7.4 (the surface pH of normal cells) about 5.2% of Zr-DFO-cys-Var3pHLIP molecules are expected to be inserted into the membranes. See Anderson M, Moshnikova A, Engelman D M, Reshetnyak Y K, Andreev O A. “Probe for the measurement of cell surface pH in vivo and ex vivo.” Proc Natl Acad Sci USA. 2016; 113(29):8177-81 (doi: 10.1073/pnas.1608247113; PubMed PMID: 27382181; PMCID: PMC4961157); and Wei D, Engelman D M, Reshetnyak Y K, Andreev O A. “Mapping pH at Cancer Cell Surfaces.” Mol Imaging Biol. 2019; 21(6):1020-5 (Epub 2019/04/17; doi: 10.1007/s11307-019-01335-4; PubMed PMID: 30989440). Since the rate constant of Zr-DFO-cys-Var3pHLIP insertion into a membrane is high (approximately 22.2 sec⁻¹; see FIG. 8), the insertion will occur even under conditions of fast blood flow and limited exposure time of Zr-DFO-cys-Var3pHLIP to acidic cancer cells.

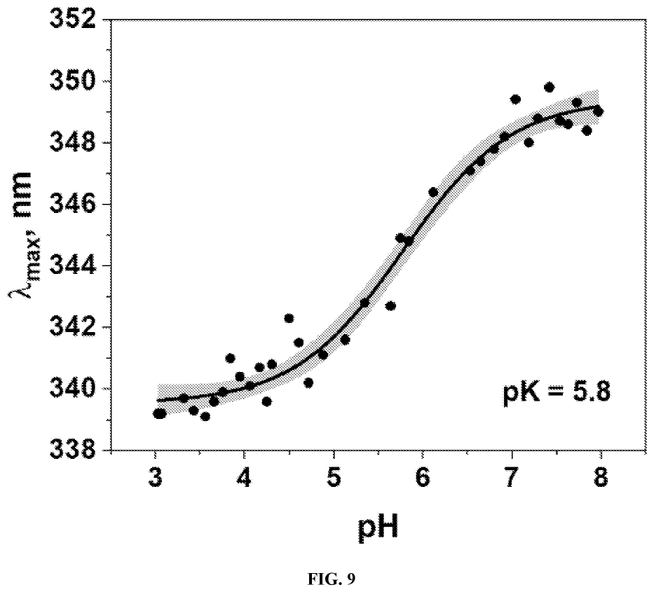

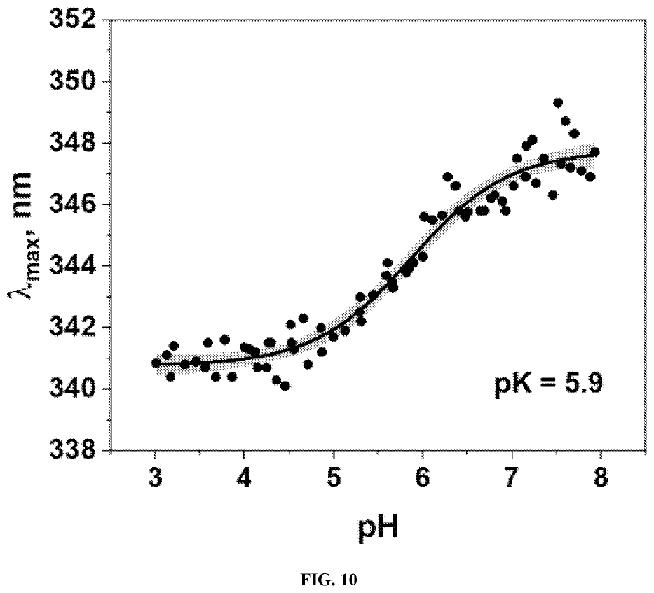

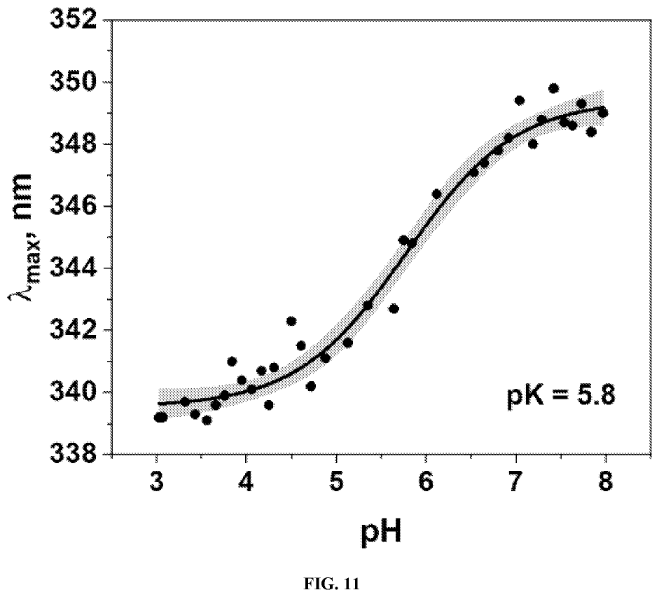

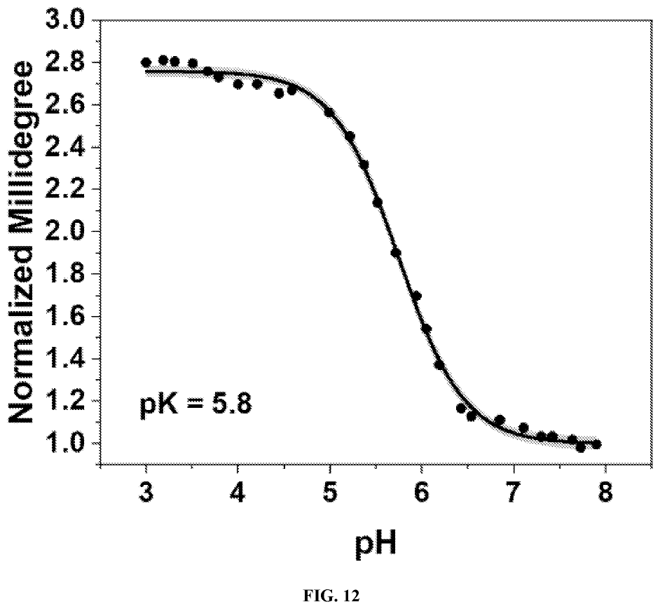

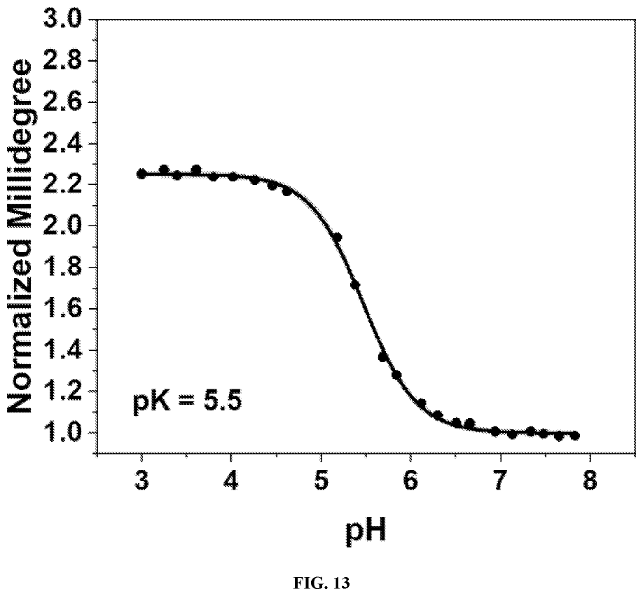

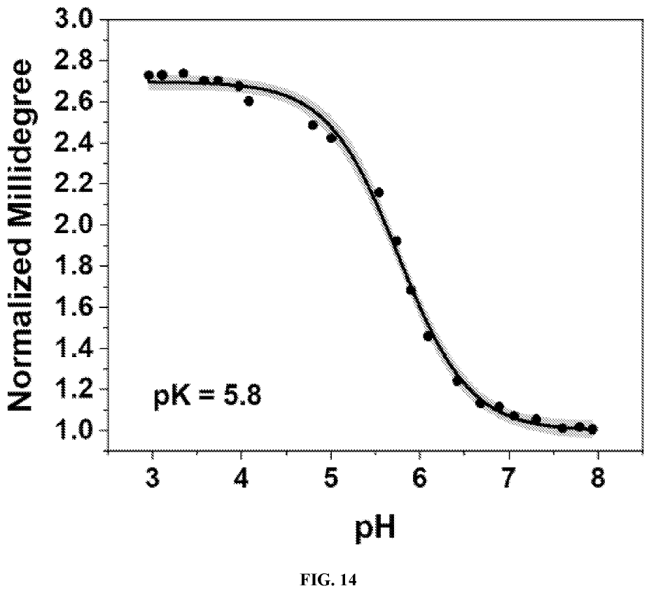

The midpoint of the transition for each of Zr-DFOsqa-lys-Var3pHLIP, Zr-DFO*-lys-Var3pHLIP, and Zr-DFO-lys-Var3pHLIP insertion into the bilayers of POPC liposomes is at about pH 5.8 for Zr-DFOsqa-lys-Var3pHLIP, about 5.9 for Zr-DFO*-lys-Var3pHLIP, and about 5.9 for Zr-DFO-lys-Var3pHLIP, as respectively calculated from graphs presented in FIG. 9, FIG. 10, and FIG. 11. pH-dependent insertions of the Zr-DFOsqa-lys-Var3pHLIP, Zr-DFO*-lys-Var3pHLIP, and Zr-DFO-lys-Var3pHLIP into the bilayers of POPC liposomes were also monitored by changes of the circular dichroism measured in millidegree indicating on coil-helix transition induced by drop of pH from 8 to 3, where the data were fitted with the Henderson-Hasselbach equation (black line with 95% confidence interval) to establish the midpoint of the transition (pK) and the data provided in FIG. 12 for Zr-DFOsqa-lys-Var3pHLIP, in FIG. 13 for Zr-DFO*-lys-Var3pHLIP, and in FIG. 14 for Zr-DFO-lys-Var3pHLIP. Zr-DFOsqa-lys-Var3pHLIP demonstrates lower affinity to the membrane at high pH compared to the other Cold-Zr-pHLIP (less of potential off targeting), while Zr-DFOsqa-lys-Var3pHLIP also inserts into membrane at low pH with pK=5.8 (please note, pK of insertion into plasma membrane of live cells is expected to be shifted to about 0.2-0.4 pH units to higher pH values of about 6.0-6.2), where such properties evidence that agents such as [⁸⁹Zr]Zr-DFOsqa-lys-Var3pHLIP will peform well PET imaging.

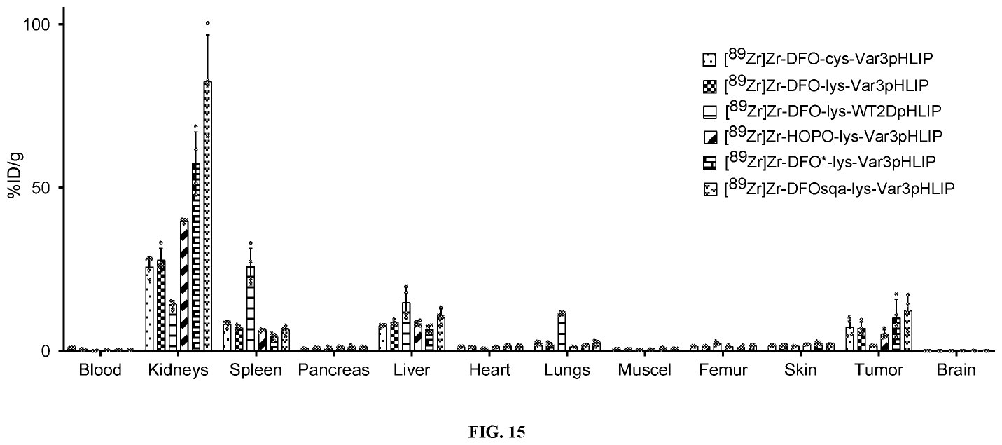

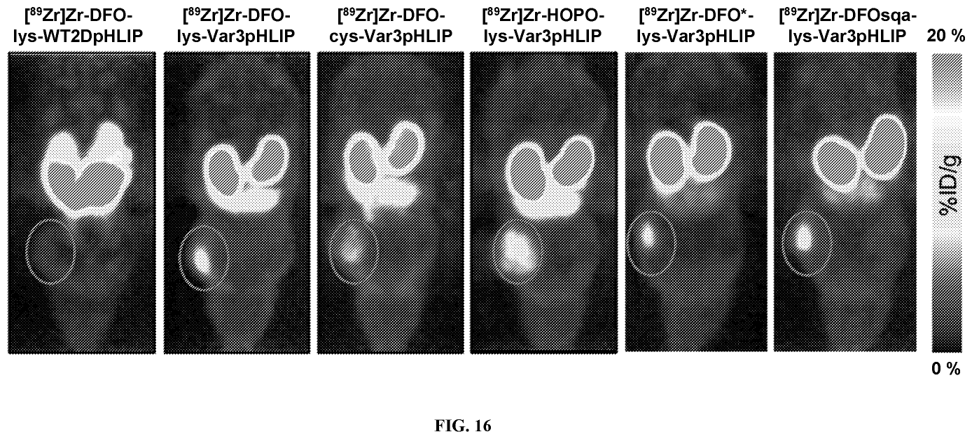

Further Mouse Model Studies

[⁸⁹Zr]Zr-DFO-lys-WT2DpHLIP, [⁸⁹Zr]Zr-DFO-cys-Var3pHLIP, [⁸⁹Zr]Zr-DFOsqa-lys-Var3pHLIP, [⁸⁹Zr]Zr-HOPO-lys-Var3pHLIP, [⁸⁹Zr]Zr-DFO*-lys-Var3pHLIP, and [⁸⁹Zr]Zr-DFO-lys-Var3pHLIP were compared for their use as PET probes for cancer. Groups of male athymic nude mice (4 mice per group) bearing orthotopic RM-1 tumor allografts were used as a model for aggressive end-stage prostate cancer. Each group of male athymic nude mice (4 mice per group) were injected with 200 μCi (1.2 nmol) of one of [⁸⁹Zr]Zr-DFO-lys-WT2DpHLIP, [⁸⁹Zr]Zr-DFO-cys-Var3pHLIP, [⁸⁹Zr]Zr-DFOsqa-lys-Var3pHLIP, [⁸⁹Zr]Zr-HOPO-lys-Var3pHLIP, [⁸⁹Zr]Zr-DFO*-lys-Var3pHLIP, and [⁸⁹Zr]Zr-DFO-lys-Var3pHLIP. The groups of mice were imaged 24, 48, and 72 h p.i. to determine the biodistribution in vivo. Tissue samples were collected for additional ex vivo biodistribution studies to confirm the PET data results. Autoradiograms and hematoxylin & eosin staining of tissue-slices were used to illuminate the conjugate's distribution inside the tumor. Tables 9 and 10 provide the ex vivo distribution in selected organs (mean % ID/g±standard deviation) at 48 p.i. for each tested complex, where such data is also graphically illustrated in FIG. 15. FIG. 16 provides coronal in vivo PET images (maximum intensity projection) at 48 h p.i., for each of the tested complexes, where the circles indicate the tumor's location (scale: percent injected dose per gram of tissue (% ID/g)). The highest tumor uptake of (12.4±4.7) % ID/g at 48 h p.i. was obtained for the [⁸⁹Zr]Zr-DFOsqa-lys-Var3pHLIP.

TABLE 9

| | [⁸⁹Zr]Zr- | [⁸⁹Zr]Zr- | [⁸⁹Zr]Zr- |
|---|---|---|---|
| | DFO-lys- | DFO-cys- | DFO-lys- |
| Tissue | WT2DpHLIP | Var3pHLIP | Var3pHLIP |
| (n = 4) | (RM-1) | (RM-1) | (RM-1) |
| Blood | 0.1 ± 003 | 1.1 ± 0.1 | 0.5 ± 0.1 |
| Kidneys | 14.2 ± 1.2 | 25.7 ± 3.0 | 27.9 ± 3.6 |
| Spleen | 25.8 ± 5.5 | 8.2 ± 1.1 | 7.2 ± 0.7 |
| Pancreas | 0.9 ± 0.4 | 0.7 ± 0.1 | 1.0 ± 0.2 |
| Liver | 14.8 ± 4.7 | 7.7 ± 0.5 | 8.7 ± 0.8 |
| Heart | 0.7 ± 0.1 | 1.3 ± 0.4 | 1.3 ± 0.03 |
| Lungs | 11.5 ± 0.4 | 2.3 ± 0.2 | 2.0 ± 0.6 |
| Muscle | 0.3 ± 0.08 | 0.6 ± 0.7 | 0.5 ± 0.1 |
| Femur | 2.3 ± 0.5 | 1.4 ± 0.1 | 1.4 ± 0.2 |
| Skin | 1.5 ± 0.2 | 1.7 ± 0.2 | 1.7 ± 0.3 |
| Tumor | 1.6 ± 0.1 | 7.3 ± 2.9 | 6.9 ± 1.9 |
| Brain | 0.03 ± 0.03 | 0.08 ± 0.01 | 0.06 ± 0.01 |

TABLE 10

| | [⁸⁹Zr]Zr- | [⁸⁹Zr]Zr- | [⁸⁹Zr]Zr- |
|---|---|---|---|
| | DFO*-lys- | HOPO-lys- | DFOsqa-lys- |
| Tissue | Var3pHLIP | Var3pHLIP | Var3pHLIP |
| (n = 4) | (RM-1) | (RM-1) | (RM-1) |
| Blood | 0.4 ± 0.09 | 0.2 ± 0.03 | 0.3 ± 0.03 |
| Kidneys | 57.5 ± 9.6 | 39.8 ± 0.7 | 82.5 ± 14.2 |
| Spleen | 4.4 ± 0.9 | 6.4 ± 0.3 | 6.4 ± 1.4 |
| Pancreas | 1.1 ± 0.4 | 1.2 ± 0.2 | 1.2 ± 0.1 |
| Liver | 6.7 ± 1.2 | 8.4 ± 0.8 | 10.8 ± 2.1 |
| Heart | 1.5 ± 0.3 | 1.2 ± 0.1 | 1.6 ± 0.1 |
| Lungs | 1.7 ± 0.2 | 1.3 ± 0.08 | 2.5 ± 0.6 |
| Muscle | 0.8 ± 0.2 | 0.5 ± 0.03 | 0.7 ± 0.09 |
| Femur | 1.1 ± 0.5 | 1.4 ± 0.3 | 1.6 ± 0.1 |
| Skin | 2.4 ± 0.5 | 2.0 ± 0.1 | 2.1 ± 0.07 |
| Tumor | 10.2 ± 5.6 | 5.2 ± 1.3 | 12.4 ± 4.7 |
| Brain | 0.07 ± 0.02 | 0.03 ± 0.01 | 0.06 ± 0.02 |

This study evidences that each of [⁸⁹Zr]Zr-DFO-cys-Var3pHLIP, [⁸⁹Zr]Zr-DFOsqa-lys-Var3pHLIP, [⁸⁹Zr]Zr-HOPO-lys-Var3pHLIP, [⁸⁹Zr]Zr-DFO*-lys-Var3pHLIP, and [⁸⁹Zr]Zr-DFO-lys-Var3pHLIP targeted the tumor. Autoradiography and hematoxylin & eosin staining of the tumor tissue revealed a correlation between its vascularization and the drug uptake. For all complexes of the present technology, a high accumulation in the kidneys' cortex (13 to 55% ID/g) was observed, most likely due to their acid-base regulatory function.

### EQUIVALENTS

While certain embodiments have been illustrated and described, a person with ordinary skill in the art, after reading the foregoing specification, can effect changes, substitutions of equivalents and other types of alterations to the compounds and complexes of the present technology as set forth herein. Each aspect and embodiment described above can also have included or incorporated therewith such variations or aspects as disclosed in regard to any or all of the other aspects and embodiments.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A compound or pharmaceutically acceptable salt thereof comprising
   a pH (low) insertion peptide (“pHLIP”) configured to localize to an extracellular environment having a pH that is lower than 7.4, wherein the pHLIP comprises a C-terminus and an N-terminus; and
   X¹ covalently attached to a heteroatom of a side chain of an amino acid residue of the pHLIP, where the amino acid residue is from 0, 1, 2, 3, 4, 5, 6, 7, 8, 9, or 10 residues from the C-terminus or the N-terminus;
   wherein
   X¹ is of Formula I, Formula II, or Formula III

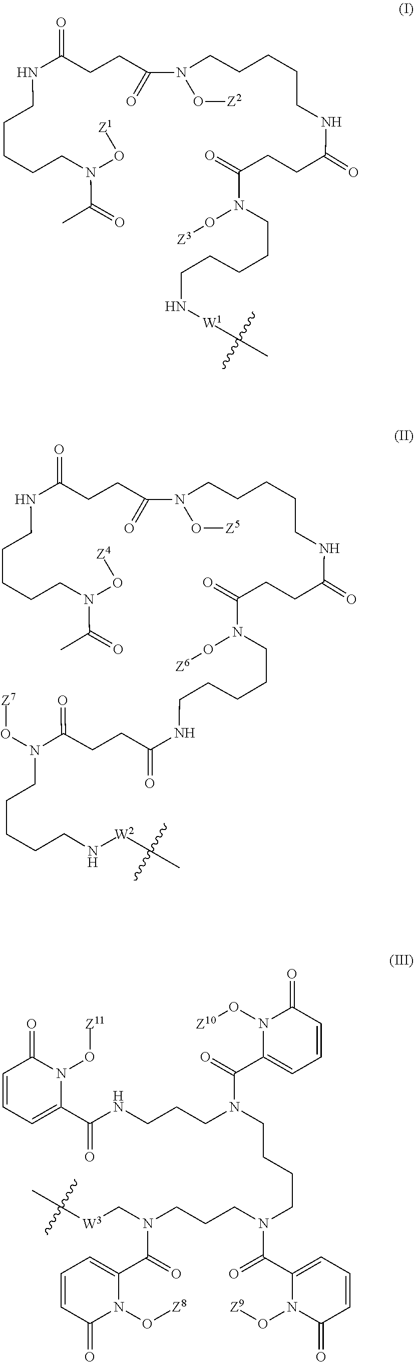

where Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and
W¹ and W² are each independently

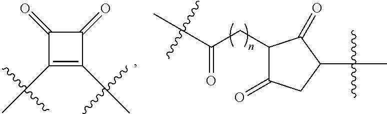

where n is 1, 2, 3, 4, 5, 6, 7, 8, 9, 10, 11, or 12,

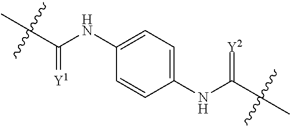

where Y¹ and Y² are each independently O or S, alkylene, alkanoylene, poly(alkylene glycol), —C(O)CH₂—((poly(alkylene glycol))-, or a bond; and
W³ is

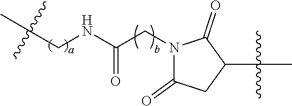

where a is 0, 1, 2, 3, 4, 5, 6, 7, 8, 9, 10, 11, or 12, and b is 1, 2, 3, 4, 5, 6, 7, 8, 9, 10, 11, or 12,

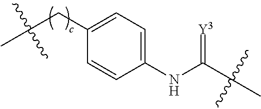

where Y³ is O or S, and c is 0, 1, 2, 3, 4, or 5,

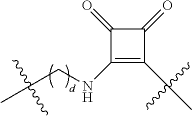

where d is 0, 1, 2, 3, 4, 5, 6, 7, 8, 9, 10, 11, or 12,

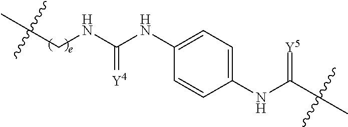

where
   e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond.
B. The compound of Paragraph A, wherein the pHLIP is configured to localize to an extracellular environment having a pH that is lower than 7.1.
C. The compound of Paragraph A or Paragraph B, wherein the pHLIP is configured to localize to a tissue comprising the extracellular environment.
D. The compound of any one of Paragraphs A-C, wherein W¹ and W² are each independently

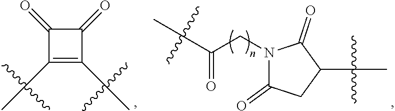

(CH₂)ₘ—, —C(O)(CH₂)ₚ—, —CH₂CH₂—(O—CH₂CH₂)_{q}—, —C(O)CH₂—(O—CH₂CH₂)ᵣ—, or a bond, where
   n is 1, 2, 3, 4, 5, 6, 7, 8, 9, 10, 11, or 12;
   m is 1, 2, 3, 4, 5, 6, 7, 8, 9, 10, 11, or 12;
   p is 1, 2, 3, 4, 5, 6, 7, 8, 9, 10, 11, or 12;
   q is 1, 2, 3, 4, 5, 6, 7, 8, 9, 10, 11, or 12; and
   r is 1, 2, 3, 4, 5, 6, 7, 8, 9, 10, 11, or 12.
E. The compound of any one of Paragraphs A-D, wherein W³ is

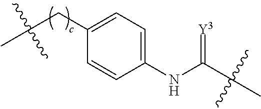

where Y³ is O or S, and c is 0, 1, 2, 3, 4, or 5,

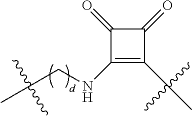

where d is 0, 1, 2, 3, 4, 5, 6, 7, 8, 9, 10, 11, or 12, —(CH₂)ₚ—, —C(O)(CH₂)_{g}—, —CH₂CH₂—(O—CH₂CH₂)ₕ—, —C(O)CH₂—(O—CH₂CH₂)ᵢ—, or a bond, where
   f is 1, 2, 3, 4, 5, 6, 7, 8, 9, 10, 11, or 12;
   g is 1, 2, 3, 4, 5, 6, 7, 8, 9, 10, 11, or 12;
   h is 1, 2, 3, 4, 5, 6, 7, 8, 9, 10, 11, or 12; and
   i is 1, 2, 3, 4, 5, 6, 7, 8, 9, 10, 11, or 12.
F. The compound of any one of Paragraphs A-E, wherein the pHLIP is

(SEQ ID NO. 239)
Ac-(D-Ala)-(D-Lys)-(D-Asp)-(D-Asp)-(D-Gln)-(D-Asn)-(D-Pro)-(D-Trp)-(D-Arg)-
(D-Ala)-(D-Tyr)-(D-Leu)-(D-Asp)-(D-Leu)-(D-Leu)-(D-Phe)-(D-Pro)-(D-
Thr)-(D-Asp)-(D-Thr)-(D-Leu)-(D-Leu)-(D-Leu)-(D-Asp)-(D-Leu)-(D-Leu)-
(D-Trp)-(D-Ala),
(SEQ ID NO. 1)
(D-Ala)-(D-Cys)-(D-Asp)-(D-Asp)-(D-Gln)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp),
(SEQ ID NO. 2)
(D-Ala)-(D-Cys)-(D-Asp)-(D-Gln)-(D-Asp)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp),
(SEQ ID NO. 3)
(D-Ala)-(D-Lys)-(D-Asp)-(D-Asp)-(D-Gln)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp),
(SEQ ID NO. 4)
(D-Ala)-(D-Lys)-(D-Asp)-(D-Gln)-(D-Asn)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp),
(SEQ ID NO. 5
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys)-(Gly),
(SEQ ID NO. 6
(D-Ala)-(D-Asp)-(D-Gln)-(D-Asp)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Cys)-(Gly),
(SEQ ID NO. 7)
(D-Ala)-(D-Asp)-(D-Asp)-(D-Gln)-(D-Asn)-(D-Pro)-(D-Trp)-(D-Arg)-(D-Ala)-(D-
Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-
Lys)-(Gly),
or
(SEQ ID NO. 8)
(D-Ala)-(D-Lys)-(D-Asp)-(D-Gln)-(D-Asn)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-
(D-Asp)-(D-Thr)-(D-Leu)-(D-Leu)-(D-Leu)-(D-Asp)-(D-Leu)-(D-Leu)-(D-
Trp)-(D-Lys)-(Gly).

G. The compound of any one of Paragraphs A-F, wherein the amino acid residue is a cysteine or lysine.
H. The compound of any one of Paragraphs A-G, wherein X¹ is covalently attached to a sulfur atom of a cysteine residue of the pHLIP or is covalently attached to a F-nitrogen atom of a lysine residue of the pHLIP.
I. The compound of any one of Paragraphs A-H, wherein the amino acid residue is a D-cysteine or D-lysine.
J. The compound of any one of Paragraphs A-I, wherein X¹ is covalently attached to a sulfur atom of a D-cysteine residue of the pHLIP or is covalently attached to a F-nitrogen atom of a D-lysine residue of the pHLIP.
K. The compound of any one of Paragraphs A-J, wherein the compound is
   Ac-(D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 240),
   (D-Ala)-(D-Cys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 9),
   (D-Ala)-(D-Cys[X¹])-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 10),
   (D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 11),
   (D-Ala)-(D-Lys[X¹])-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 12),
   (D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X¹])-(Gly) (SEQ ID NO. 13),
   (D-Ala)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X¹])-(Gly) (SEQ ID NO. 14),
   (D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X¹])-(Gly) (SEQ ID NO. 15),
   (D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X¹])-(Gly) (SEQ ID NO. 16), or
   a pharmaceutically acceptable salt of any one thereof.
L. The compound of any one of Paragraphs A-K, wherein the compound is

(SEQ ID NO. 240)
Ac-(D-Ala)-(D-Lys[X¹])-(D-Asp)-(D-Asp)-(D-Gln)-
(D-Asn)-(D-Pro)-(D-Trp)-(D-Arg)-(D-Ala)-(D-Tyr)-
(D-Leu)-(D-Asp)-(D-Leu)-(D-Leu)-(D-Phe)-(D-Pro)-
(D-Thr)-(D-Asp)-(D-Thr)-(D-Leu)-(D-Leu)-(D-Leu)-
(D-Asp)-(D-Leu)-(D-Leu)-(D-Trp)-(D-Ala)

or a pharmaceutically acceptable salt thereof, or
(D-Ala)-(D-Cys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 9) or a pharmaceutically acceptable salt thereof.
M. The compound of any one of Paragraphs A-L, wherein X¹ is of Formula Ia, Formula Ib, or Formula IIIa

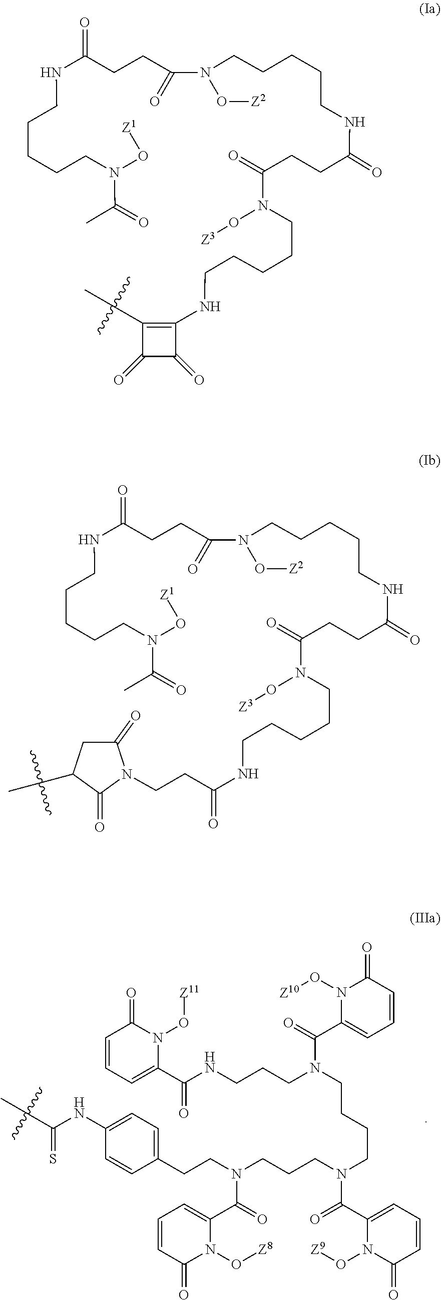

N. A complex comprising the compound of any one of Paragraphs A-M and a radionuclide chelated by the X¹ group.
O. The complex of Paragraph N, wherein X² is the X¹ and the radionuclide together, and wherein X² is represented by Formula IV, Formula V, or Formula VI

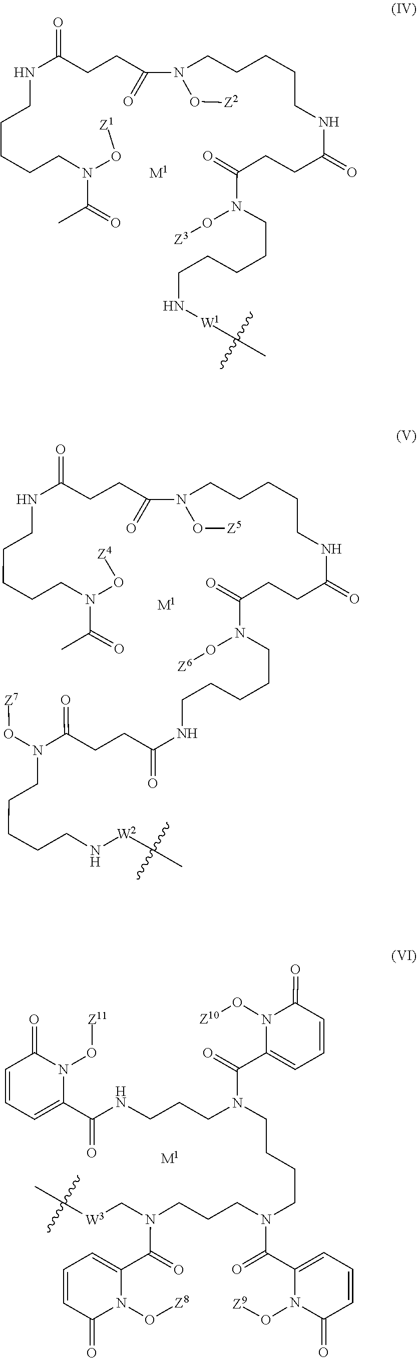

wherein
M¹ is independently at each occurrence the radionuclide (e.g., ⁸⁹Zr⁴⁺, ⁶⁷Ga³⁺ or ⁶⁸Ga³⁺)
Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and
W¹ and W² are each independently

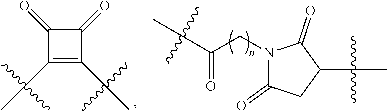

where n is 1, 2, 3, 4, 5, 6, 7, 8, 9, 10, 11, or 12,

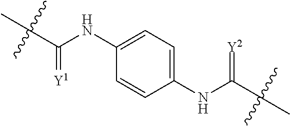

where Y¹ and Y² are each independently O or S, alkylene, alkanoylene, poly(alkylene glycol), —C(O)CH₂-(poly(alkylene glycol))-, or a bond; and
W³ is

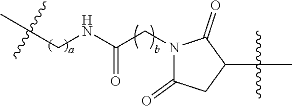

where a is 0, 1, 2, 3, 4, 5, 6, 7, 8, 9, 10, 11, or 12, and bis 1, 2, 3, 4, 5, 6, 7, 8, 9, 10, 11, or 12,

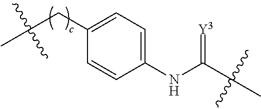

where Y³ is O or S, and c is 0, 1, 2, 3, 4, or 5

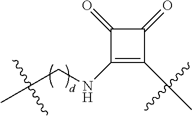

where d is 0, 1, 2, 3, 4, 5, 6, 7, 8, 9, 10, 11, or 12,

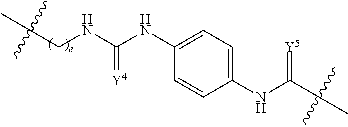

where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond.
P. The complex of Paragraph O, wherein W¹ and W² are each independently

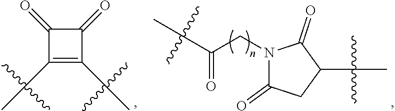

—(CH₂)ₘ—, —C(O)(CH₂)ₚ—, —CH₂CH₂—(O—CH₂CH₂)_{q}—, —C(O)CH₂—(O—CH₂CH₂)ᵣ—, or a bond, where
   n is 1, 2, 3, 4, 5, 6, 7, 8, 9, 10, 11, or 12;
   m is 1, 2, 3, 4, 5, 6, 7, 8, 9, 10, 11, or 12;
   p is 1, 2, 3, 4, 5, 6, 7, 8, 9, 10, 11, or 12;
   q is 1, 2, 3, 4, 5, 6, 7, 8, 9, 10, 11, or 12; and
   r is 1, 2, 3, 4, 5, 6, 7, 8, 9, 10, 11, or 12.
Q. The complex of Paragraph O or Paragraph P, wherein W³ is

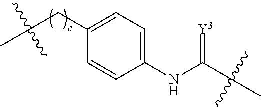

where Y³ is O or S, and c is 0, 1, 2, 3, 4, or 5,

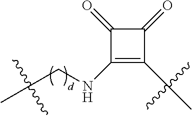

where d is 0, 1, 2, 3, 4, 5, 6, 7, 8, 9, 10, 11, or 12, —(CH₂)—, —C(O)(CH₂)_{g}—, —CH₂CH₂—(O—CH₂CH₂)ₕ—, —C(O)CH₂—(O—CH₂CH₂)—, or a bond, where
   f is 1, 2, 3, 4, 5, 6, 7, 8, 9, 10, 11, or 12;
   g is 1, 2, 3, 4, 5, 6, 7, 8, 9, 10, 11, or 12;
   h is 1, 2, 3, 4, 5, 6, 7, 8, 9, 10, 11, or 12; and
   i is 1, 2, 3, 4, 5, 6, 7, 8, 9, 10, 11, or 12.
R. The complex of any one of Paragraphs O-Q, wherein X² is of Formula IVa, Formula IVb, or Formula VIa

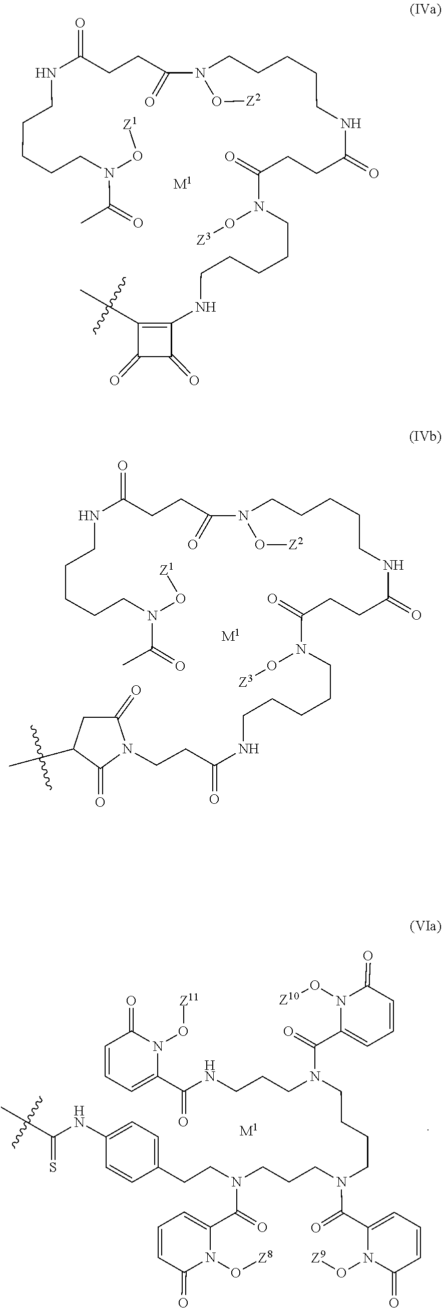

S. The complex of any one of Paragraphs O-R, wherein the complex is
   Ac-(D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 241),
   (D-Ala)-(D-Cys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 17),
   (D-Ala)-(D-Cys[X²])-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 18),
   (D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 19),
   (D-Ala)-(D-Lys[X²])-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 20),
   (D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X²])-(Gly) (SEQ ID NO. 21),
   (D-Ala)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X²])-(Gly) (SEQ ID NO. 22),
   (D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X²])-(Gly) (SEQ ID NO. 23),
   (D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X²])-(Gly) (SEQ ID NO. 24), or
   a pharmaceutically acceptable salt of any one thereof.
T. The complex of any one of Paragraphs O-S, wherein the complex is
   Ac-(D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 241) or a pharmaceutically acceptable salt thereof; or
   (D-Ala)-(D-Cys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 17) or a pharmaceutically acceptable salt thereof.
U. A composition comprising a compound of any one of Paragraphs A-M and a pharmaceutically acceptable carrier.
V. A composition comprising a complex of any one of Paragraphs N-T and a pharmaceutically acceptable carrier.
W. A pharmaceutical composition for detecting a tissue comprising an extracellular environment having a pH that is lower than 7.4, the pharmaceutical composition comprising an effective amount of a complex of any one of Paragraphs N-T and a pharmaceutically acceptable carrier.
X. The pharmaceutical composition of Paragraph W, wherein the tissue comprises one or more of the group consisting of an atherosclerotic plaque, ischemic myocardium, a tissue impacted by stroke, an inflamed tissue, and a cancer tissue.
Y. The pharmaceutical composition of Paragraph X, wherein the cancer tissue is selected from the group consisting of breast cancer, a brain tumor, prostate cancer, melanoma, and a metastatic cancer of any one thereof.
Z. The pharmaceutical composition of Paragraph X or Paragraph Y, wherein the cancer tissue is a brain tumor.
AA. A method for detecting diseased tissue in a subject in need thereof comprising
   (a) administering an effective amount of a complex of any one of Paragraphs N-T to the subject; and
   (b) detecting the presence of a tissue comprising an extracellular environment having a pH that is lower than 7.4 in the subject by detecting radioactive levels emitted by the complex that are higher than a reference value, thereby detecting the diseased tissue.
AB. The method of Paragraph AA, wherein the radioactive levels emitted by the complex are detected using positron emission tomography or single photon emission computed tomography.
AC. The method of Paragraph AA or Paragraph AB, wherein the subject is diagnosed with, or is suspected of having, an atherosclerotic plaque, ischemic myocardium, a tissue impacted by stroke, an inflamed tissue, or cancer.
AD. The method of Paragraph AC, wherein the cancer is selected from the group consisting of breast cancer, a brain tumor, prostate cancer, melanoma, and a metastatic cancer of any one thereof.
AE. The method of Paragraph AC or Paragraph AD, wherein the cancer is a brain tumor.
AF. The method of any one of Paragraphs AA-AE, wherein the complex is administered into the cerebral spinal fluid or blood of the subject.
AG. The method of any one of Paragraphs AA-AF, wherein the complex is administered intravenously, intramuscularly, intraarterially, intrathecally, intracapsularly, intradermally, intraperitoneally, intralesionally, transtracheally, subcutaneously, intracerebroventricularly, orally, intranasally, rectally, topically, or via inhalation.
AH. The method of any one of Paragraphs AA-AG, wherein the radioactive levels emitted by the complex are detected between 4 to 24 hours after the complex is administered.
AI. The method of any one of Paragraphs AA-AH, wherein the radioactive levels emitted by the complex are expressed as the percentage injected dose per gram tissue (% ID/g).
AJ. The method of any one of Paragraphs AA-AI, wherein a ratio of radioactive levels between the solid tumor and normal tissue is about 2:1 to about 100:1.
AK. A method for detecting solid tumors in a subject in need thereof comprising
   (a) administering an effective amount of a complex of any one of Paragraphs N-T to the subject; and
   (b) detecting the presence of one or more solid tumors in the subject by detecting radioactive levels emitted by the complex that are higher than a reference value.
AL. The method of Paragraph AK, wherein the radioactive levels emitted by the complex are detected using positron emission tomography or single photon emission computed tomography.
AM. The method of Paragraph AK or Paragraph AL, wherein the one or more solid tumors of a cancer selected from the group consisting of breast cancer, a brain tumor, prostate cancer, melanoma, and a metastatic cancer of any one thereof.
AN. The method of any one of Paragraphs AK-AM, wherein the one or more solid tumors comprise a brain tumor.
AO. The method of any one of Paragraphs AK-AN, wherein the complex is administered into the cerebral spinal fluid or blood of the subject.
AP. The method of any one of Paragraphs AK-AO, wherein the complex is administered intravenously, intramuscularly, intraarterially, intrathecally, intracapsularly, intradermally, intraperitoneally, intralesionally, transtracheally, subcutaneously, intracerebroventricularly, orally, intranasally, rectally, topically, or via inhalation.
AQ. The method of any one of Paragraphs AK-AP, wherein the radioactive levels emitted by the complex are detected between 4 to 24 hours after the complex is administered.
AR. The method of any one of Paragraphs AK-AQ, wherein the radioactive levels emitted by the complex are expressed as the percentage injected dose per gram tissue (% ID/g).
AS. The method of any one of Paragraphs AK-AR, wherein a ratio of radioactive levels between the solid tumor and normal tissue is about 2:1 to about 100:1.
AT. A kit comprising a compound of any one of Paragraphs A-M and instructions for use.
AU. The kit of Paragraph AT, wherein the instructions for use comprise instructions for generating a complex of any one of Paragraphs N-T.
AV. The kit of Paragraph AT or Paragraph AU, wherein the instructions for use comprise instructions for performing a method of any one of Paragraphs AA-AJ.
AW. The kit of any one of Paragraphs AT-AV, wherein the instructions for use comprise instructions for performing a method of any one of Paragraphs AK-AS.

Other embodiments are set forth in the following claims, along with the full scope of equivalents to which such claims are entitled.

1. A compound or pharmaceutically acceptable salt thereof comprising
a pH (low) insertion peptide (“pHLIP”) configured to localize to an extracellular environment having a pH that is lower than 7.4, wherein the pHLIP comprises a C-terminus and an N-terminus; and
X¹ covalently attached to a heteroatom of a side chain of an amino acid residue of the pHLIP, where the amino acid residue is from 0, 1, 2, 3, 4, 5, 6, 7, 8, 9, or 10 residues from the C-terminus or the N-terminus;
wherein
X¹ is of Formula I, Formula II, or Formula III 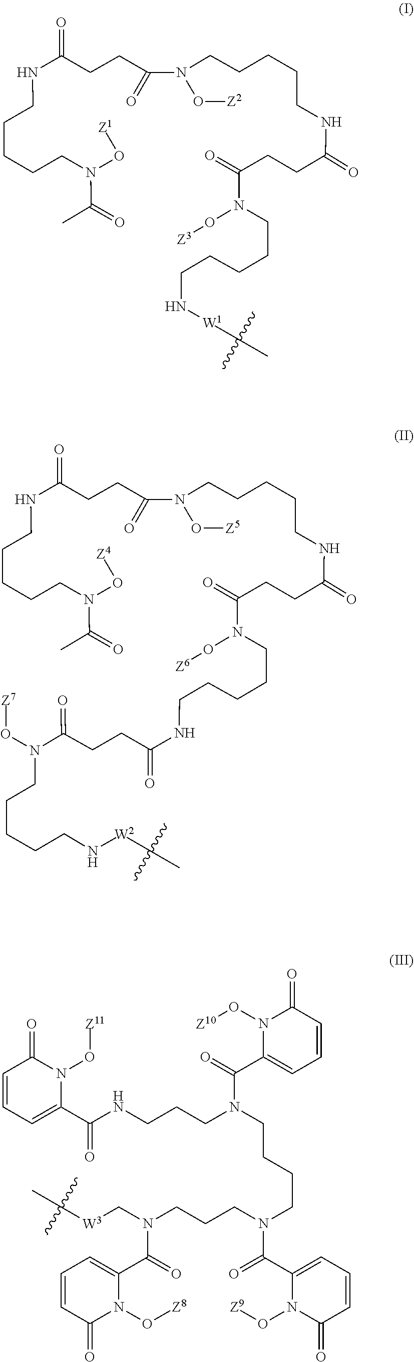
where Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and
W¹ and W² are each independently 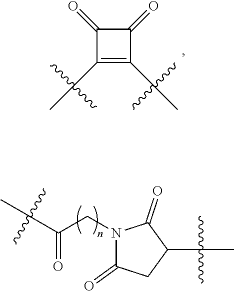
where n is 1, 2, 3, 4, 5, 6, 7, 8, 9, 10, 11, or 12, 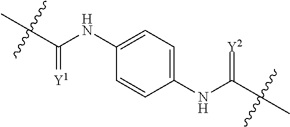
where Y¹ and Y² are each independently O or S, alkylene, alkanoylene, poly(alkylene glycol), —C(O)CH₂—((poly(alkylene glycol))-, or a bond; and
W³ is 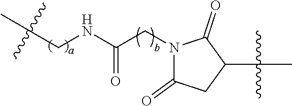
where a is 0, 1, 2, 3, 4, 5, 6, 7, 8, 9, 10, 11, or 12, and b is 1, 2, 3, 4, 5, 6, 7, 8, 9, 10, 11, or 12, 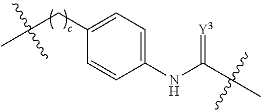
where Y³ is O or S, and c is 0, 1, 2, 3, 4, or 5, 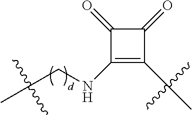
where d is 0, 1, 2, 3, 4, 5, 6, 7, 8, 9, 10, 11, or 12, 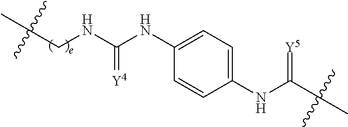
where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond.

2. The compound of claim 1, wherein the pHLIP is configured to localize to an extracellular environment having a pH that is lower than 7.1.

3. The compound of claim 1, wherein the pHLIP is configured to localize to a tissue comprising the extracellular environment.

4. The compound of claim 1, wherein W¹ and W² are each independently 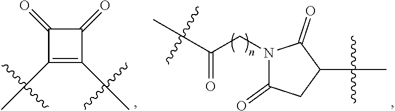
—(CH₂)ₘ, C(O)(CH₂)—, —CH₂CH₂—(O—CH₂CH₂)_{q}—, —C(O)CH₂—(O—CH₂CH₂)ᵣ—, or a bond, where
n is 1, 2, 3, 4, 5, 6, 7, 8, 9, 10, 11, or 12;
m is 1, 2, 3, 4, 5, 6, 7, 8, 9, 10, 11, or 12;
p is 1, 2, 3, 4, 5, 6, 7, 8, 9, 10, 11, or 12;
q is 1, 2, 3, 4, 5, 6, 7, 8, 9, 10, 11, or 12; and
r is 1, 2, 3, 4, 5, 6, 7, 8, 9, 10, 11, or 12.

5. The compound of claim 1, wherein W³ is 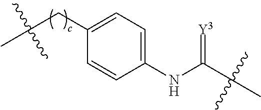
where Y³ is O or S, and c is 0, 1, 2, 3, 4, or 5, 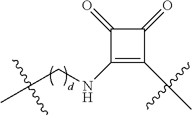
where d is 0, 1, 2, 3, 4, 5, 6, 7, 8, 9, 10, 11, or 12, —(CH₂)ᵣ—, —C(O)(CH₂)_{g}—, —CH₂CH₂—(O—CH₂CH₂)ₕ—, —C(O)CH₂—(O—CH₂CH₂)ᵣ—, or a bond, where
f is 1, 2, 3, 4, 5, 6, 7, 8, 9, 10, 11, or 12;
g is 1, 2, 3, 4, 5, 6, 7, 8, 9, 10, 11, or 12;
h is 1, 2, 3, 4, 5, 6, 7, 8, 9, 10, 11, or 12; and
i is 1, 2, 3, 4, 5, 6, 7, 8, 9, 10, 11, or 12.

6. The compound of claim 1, wherein the pHLIP is
(SEQ ID NO. 239)
Ac-(D-Ala)-(D-Lys)-(D-Asp)-(D-Asp)-(D-Gln)-(D-Asn)-(D-Pro)-(D-Trp)-(D-Arg)-(D-
Ala)-(D-Tyr)-(D-Leu)-(D-Asp)-(D-Leu)-(D-Leu)-(D-Phe)-(D-Pro)-(D-Thr)-(D-
Asp)-(D-Thr)-(D-Leu)-(D-Leu)-(D-Leu)-(D-Asp)-(D-Leu)-(D-Leu)-(D-Trp)-(D-
Ala),
(SEQ ID NO. 1)
(D-Ala)-(D-Cys)-(D-Asp)-(D-Asp)-(D-Gln)-(D-Asn)-(D-Pro)-(D-Trp)-(D-Arg)-(D-Ala)-
(D-Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp),
(SEQ ID NO. 2)
(D-Ala)-(D-Cys)-(D-Asp)-(D-Gln)-(D-Asp)-(D-Asn)-(D-Pro)-(D-Trp)-(D-Arg)-(D-Ala)-
(D-Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp),
(SEQ ID NO. 3)
(D-Ala)-(D-Lys)-(D-Asp)-(D-Asp)-(D-Gln)-(D-Asn)-(D-Pro)-(D-Trp)-(D-Arg)-(D-Ala)-
(D-Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp),
(SEQ ID NO. 4)
(D-Ala)-(D-Lys)-(D-Asp)-(D-Gln)-(D-Asn)-(D-Asn)-(D-Pro)-(D-Trp)-(D-Arg)-(D-Ala)-
(D-Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp),
(SEQ ID NO. 5
(D-Ala)-(D-Asp)-(D-Asp)-(D-Gln)-(D-Asn)-(D-Pro)-(D-Trp)-(D-Arg)-(D-Ala)-(D-Tyr)-
(D-Leu)-(D-Asp)-(D-Leu)-(D-Leu)-(D-Phe)-(D-Pro)-(D-Thr)-(D-Asp)-(D-Thr)-
(D-Leu)-(D-Leu)-(D-Leu)-(D-Asp)-(D-Leu)-(D-Leu)-(D-Trp)-(D-Cys)-(Gly),
(SEQ ID NO. 6
(D-Ala)-(D-Asp)-(D-Gln)-(D-Asp)-(D-Asn)-(D-Pro)-(D-Trp)-(D-Arg)-(D-Ala)-(D-Tyr)-
(D-Leu)-(D-Asp)-(D-Leu)-(D-Leu)-(D-Phe)-(D-Pro)-(D-Thr)-(D-Asp)-(D-Thr)-
(D-Leu)-(D-Leu)-(D-Leu)-(D-Asp)-(D-Leu)-(D-Leu)-(D-Trp)-(D-Cys)-(Gly),
(SEQ ID NO. 7)
(D-Ala)-(D-Asp)-(D-Asp)-(D-Gln)-(D-Asn)-(D-Pro)-(D-Trp)-(D-Arg)-(D-Ala)-(D-Tyr)-
(D-Leu)-(D-Asp)-(D-Leu)-(D-Leu)-(D-Phe)-(D-Pro)-(D-Thr)-(D-Asp)-(D-Thr)-
(D-Leu)-(D-Leu)-(D-Leu)-(D-Asp)-(D-Leu)-(D-Leu)-(D-Trp)-(D-Lys)-(Gly),
or
(SEQ ID NO. 8)
(D-Ala)-(D-Lys)-(D-Asp)-(D-Gln)-(D-Asn)-(D-Asn)-(D-Pro)-(D-Trp)-(D-Arg)-(D-Ala)-
(D-Tyr)-(D-Leu)-(D-Asp)-(D-Leu)-(D-Leu)-(D-Phe)-(D-Pro)-(D-Thr)-(D-Asp)-
(D-Thr)-(D-Leu)-(D-Leu)-(D-Leu)-(D-Asp)-(D-Leu)-(D-Leu)-(D-Trp)-(D-Lys)-
(Gly).

7. The compound of claim 1, wherein the amino acid residue is a cysteine or lysine.

8. The compound of claim 1, wherein X¹ is covalently attached to a sulfur atom of a cysteine residue of the pHLIP or is covalently attached to a F-nitrogen atom of a lysine residue of the pHLIP.

9. The compound of claim 1, wherein the amino acid residue is a D-cysteine or D-lysine.

10. The compound of claim 1, wherein X¹ is covalently attached to a sulfur atom of a D-cysteine residue of the pHLIP or is covalently attached to a F-nitrogen atom of a D-lysine residue of the pHLIP.

11. The compound of claim 1, wherein the compound is
Ac-(D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 240),
(D-Ala)-(D-Cys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 9),
(D-Ala)-(D-Cys[X¹])-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 10),
(D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 11),
(D-Ala)-(D-Lys[X¹])-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 12),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X¹])-(Gly) (SEQ ID NO. 13),
(D-Ala)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X¹])-(Gly) (SEQ ID NO. 14),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X¹])-(Gly) (SEQ ID NO. 15),
(D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X¹])-(Gly) (SEQ ID NO. 16), or
a pharmaceutically acceptable salt of any one thereof.

12. The compound of claim 1, wherein the compound is
Ac-(D-Ala)-(D-Lys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 240) or a pharmaceutically acceptable salt thereof, or
(D-Ala)-(D-Cys[X¹])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 9) or a pharmaceutically acceptable salt thereof.

13. The compound of claim 1, wherein X¹ is of Formula Ia, Formula Ib, or Formula IIIa

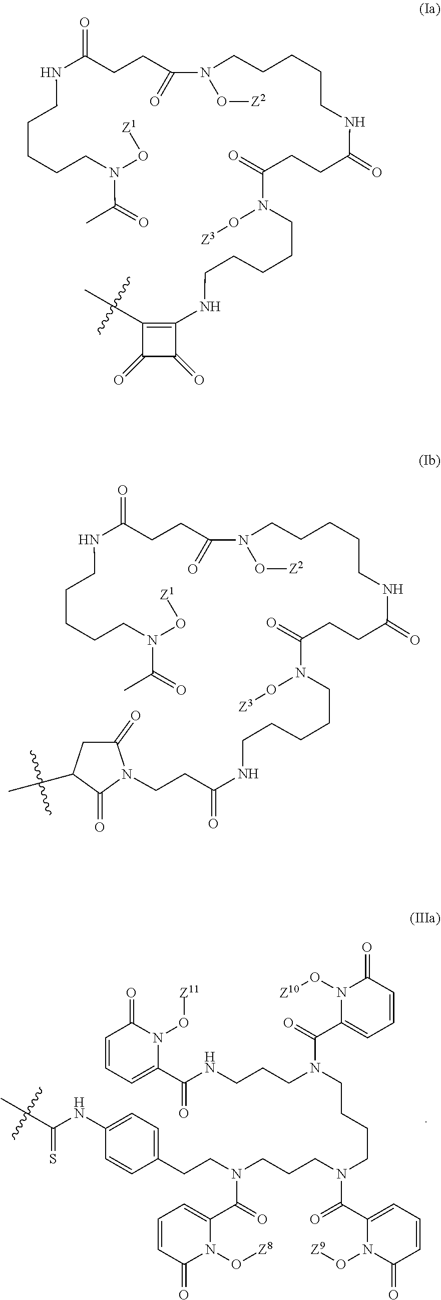

14. A complex comprising the compound of claim 1 and a radionuclide chelated by the X¹ group.

15. The complex of claim 14, wherein X² is the X¹ and the radionuclide together, and wherein X² is represented by Formula IV, Formula V, or Formula VI 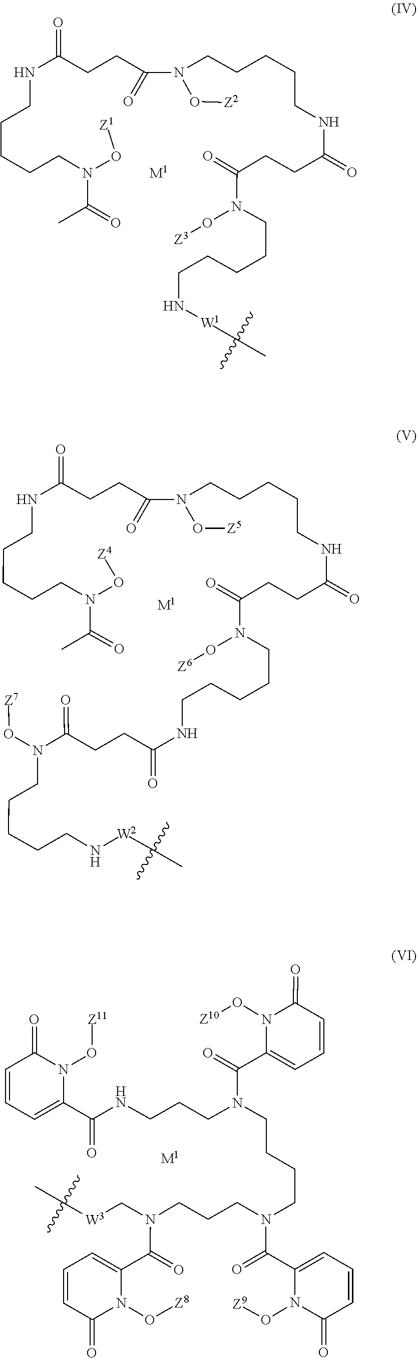
wherein
M¹ is independently at each occurrence the radionuclide (e.g., ⁸⁹Zr⁴⁺, ⁶⁷Ga³⁺, or ⁶⁸Ga³⁺)
Z¹, Z², Z³, Z⁴, Z⁵, Z⁶, Z⁷, Z⁸, Z⁹, Z¹⁰, and Z¹¹ are each independently a lone pair of electrons (i.e., providing an oxygen anion) or H; and
W¹ and W² are each independently 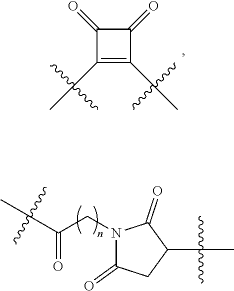
where n is 1, 2, 3, 4, 5, 6, 7, 8, 9, 10, 11, or 12, 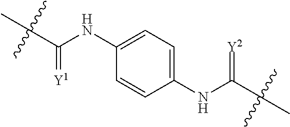
where Y¹ and Y² are each independently O or S, alkylene, alkanoylene, poly(alkylene glycol), —C(O)CH₂-(poly(alkylene glycol))-, or a bond; and
W³ is 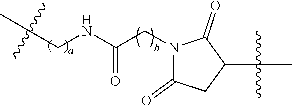
where a is 0, 1, 2, 3, 4, 5, 6, 7, 8, 9, 10, 11, or 12, and bis 1, 2, 3, 4, 5, 6, 7, 8, 9, 10, 11, or 12, 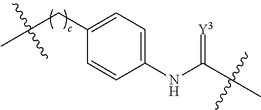
where Y³ is O or S, and c is 0, 1, 2, 3, 4, or 5, 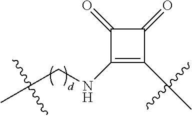
where d is 0, 1, 2, 3, 4, 5, 6, 7, 8, 9, 10, 11, or 12, 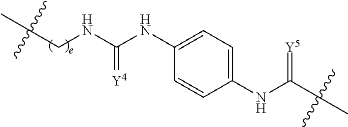
where e is 1, 2, 3, 4, 5, 6, 7, 8, 9, 10, 11, or 12, and Y⁴ and Y⁵ are each independently O or S, alkylene, alkanoylene, poly(alkylene glycol), —C(O)CH₂-(poly(alkylene glycol))-, or a bond.

16.-18. (canceled)

19. The complex of claim 15, wherein the complex is
Ac-(D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D- Leu)-(D-Trp)-(D-Ala) (SEQ ID NO. 241),
(D-Ala)-(D-Cys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 17),
(D-Ala)-(D-Cys[X²])-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 18),
(D-Ala)-(D-Lys[X²])-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 19),
(D-Ala)-(D-Lys[X²])-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)- (D-Trp) (SEQ ID NO. 20),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X²])-(Gly) (SEQ ID NO. 21),
(D-Ala)-(D-Asp)-(D-Gln)-(D-Asp)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Cys[X²])-(Gly) (SEQ ID NO. 22),
(D-Ala)-(D-Asp)-(D-Asp)-(D-Gln)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X²])-(Gly) (SEQ ID NO. 23),
(D-Ala)-(D-Lys)-(D-Asp)-(D-Gln)-(D-Asn)-(D-Asn)-(D-Pro)-(D-Trp)-(D-Arg)-(D-Ala)-(D-Tyr)-(D-Leu)-(D-Asp)-(D-Leu)-(D-Leu)-(D-Phe)-(D-Pro)-(D-Thr)-(D-Asp)-(D-Thr)-(D-Leu)-(D-Leu)-(D-Leu)-(D-Asp)-(D-Leu)-(D-Leu)-(D-Trp)-(D-Lys[X²])-(Gly) (SEQ ID NO. 24), or
a pharmaceutically acceptable salt of any one thereof.

20. (canceled)

21. A composition comprising a compound of claim 1 and a pharmaceutically acceptable carrier.

22. A composition comprising a complex of claim 14 and a pharmaceutically acceptable carrier.

23. A pharmaceutical composition for detecting a tissue comprising an extracellular environment having a pH that is lower than 7.4, the pharmaceutical composition comprising an effective amount of a complex of claim 14 and a pharmaceutically acceptable carrier.

24.-26. (canceled)

27. A method for detecting diseased tissue in a subject in need thereof comprising
(a) administering an effective amount of a complex of claim 14 to the subject; and
(b) detecting the presence of a tissue comprising an extracellular environment having a pH that is lower than 7.4 in the subject by detecting radioactive levels emitted by the complex that are higher than a reference value, thereby detecting the diseased tissue.

28.-36. (canceled)

37. A method for detecting solid tumors in a subject in need thereof comprising
(a) administering an effective amount of a complex of claim 14 to the subject; and
(b) detecting the presence of one or more solid tumors in the subject by detecting radioactive levels emitted by the complex that are higher than a reference value.

38.-45. (canceled)

46. A kit comprising a compound of claim 1 and instructions for use.

47.-49. (canceled)


SEQUENCE LISTING (244 sequences)

SEQ ID NO: 1 - protein, 27 aa, Artificial Sequence
  Description of Artificial Sequence: Synthetic peptide
  MOD_RES (1)..(27): D-amino acid
ACDDQNPWRAYLDLLFPTDTLLLDLLW

SEQ ID NO: 2 - protein, 27 aa, Artificial Sequence
  Description of Artificial Sequence: Synthetic peptide
  MOD_RES (1)..(27): D-amino acid
ACDQDNPWRAYLDLLFPTDTLLLDLLW

SEQ ID NO: 3 - protein, 27 aa, Artificial Sequence
  Description of Artificial Sequence: Synthetic peptide
  MOD_RES (1)..(27): D-amino acid
AKDDQNPWRAYLDLLFPTDTLLLDLLW

SEQ ID NO: 4 - protein, 27 aa, Artificial Sequence
  Description of Artificial Sequence: Synthetic peptide
  MOD_RES (1)..(27): D-amino acid
AKDQNNPWRAYLDLLFPTDTLLLDLLW

SEQ ID NO: 5 - protein, 28 aa, Artificial Sequence
  Description of Artificial Sequence: Synthetic peptide
  MOD_RES (1)..(27): D-amino acid
ADDQNPWRAYLDLLFPTDTLLLDLLWCG

SEQ ID NO: 6 - protein, 28 aa, Artificial Sequence
  Description of Artificial Sequence: Synthetic peptide
  MOD_RES (1)..(27): D-amino acid
ADQDNPWRAYLDLLFPTDTLLLDLLWCG

SEQ ID NO: 7 - protein, 28 aa, Artificial Sequence
  Description of Artificial Sequence: Synthetic peptide
  MOD_RES (1)..(27): D-amino acid
ADDQNPWRAYLDLLFPTDTLLLDLLWKG

SEQ ID NO: 8 - protein, 29 aa, Artificial Sequence
  Description of Artificial Sequence: Synthetic peptide
  MOD_RES (1)..(28): D-amino acid
AKDQNNPWRAYLDLLFPTDTLLLDLLWKG

SEQ ID NO: 9 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (2)..(2): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (2)..(2): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (2)..(2): CONT. FROM ABOVE: through a covalent linker with a heteroatom of a side chain of an amino acid residue of the peptide sequence
  See specification as filed for detailed description of substitutions and preferred embodiments
ACDDQNPWRAYLDLLFPTDTLLLDLLW

SEQ ID NO: 10 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25 -pentaazatriacontan-30-yl)amino group covalently associated by a bond or through a covalent linker with a heteroatom of a side chain of an amino acid residue of the peptide sequence, a
  MOD_RES (2)..(2): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (2)..(2): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (2)..(2): CONT. FROM ABOVE: through a covalent linker with a heteroatom of a side chain of an amino acid residue of the peptide sequence
  See specification as filed for detailed description of substitutions and preferred embodiments
ACDQDNPWRAYLDLLFPTDTLLLDLLW

SEQ ID NO: 11 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (2)..(2): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (2)..(2): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (2)..(2): CONT. FROM ABOVE: through a covalent linker with a heteroatom of a side chain of an amino acid residue of the peptide sequence
  See specification as filed for detailed description of substitutions and preferred embodiments
AKDDQNPWRAYLDLLFPTDTLLLDLLW

SEQ ID NO: 12 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (2)..(2): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (2)..(2): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (2)..(2): CONT. FROM ABOVE: through a covalent linker with a heteroatom of a side chain of an amino acid residue of the peptide sequence
  See specification as filed for detailed description of substitutions and preferred embodiments
AKDQNNPWRAYLDLLFPTDTLLLDLLW

SEQ ID NO: 13 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (27)..(27): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (27)..(27): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (27)..(27): CONT. FROM ABOVE: through a covalent linker with a heteroatom of a side chain of an amino acid residue of the peptide sequence
  See specification as filed for detailed description of substitutions and preferred embodiments
ADDQNPWRAYLDLLFPTDTLLLDLLWCG

SEQ ID NO: 14 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (27)..(27): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (27)..(27): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (27)..(27): CONT. FROM ABOVE: through a covalent linker with a heteroatom of a side chain of an amino acid residue of the peptide sequence
  See specification as filed for detailed description of substitutions and preferred embodiments
ADQDNPWRAYLDLLFPTDTLLLDLLWCG

SEQ ID NO: 15 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (27)..(27): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (27)..(27): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (27)..(27): CONT. FROM ABOVE: through a covalent linker with a heteroatom of a side chain of an amino acid residue of the peptide sequence
  See specification as filed for detailed description of substitutions and preferred embodiments
ADDQNPWRAYLDLLFPTDTLLLDLLWKG

SEQ ID NO: 16 - protein, 29 aa, Artificial Sequence
  Description of Artificial Sequence: Synthetic peptide
  MOD_RES (1)..(28): D-amino acid
  MOD_RES (28)..(28): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (28)..(28): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (28)..(28): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15-tetraazahexadecyl group covalently associated by a bond or
  MOD_RES (28)..(28): CONT. FROM ABOVE: through a covalent linker with a heteroatom of a side chain of an amino acid residue of the peptide sequence
  See specification as filed for detailed description of substitutions and preferred embodiments
AKDQNNPWRAYLDLLFPTDTLLLDLLWKG

SEQ ID NO: 17 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (2)..(2): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (2)..(2): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (2)..(2): CONT. FROM ABOVE: -dihydropyridine-2-carbonyl)- 16-oxo-2,6,11,15 -tetraazahexadecyl chelating a radionuclide where the group is group covalently associated by a bond or through a covalent linker with a heteroatom of a side chain of an amino acid
  MOD_RES (2)..(2): CONT. FROM ABOVE: residue of the peptide sequence
  See specification as filed for detailed description of substitutions and preferred embodiments
ACDDQNPWRAYLDLLFPTDTLLLDLLW

SEQ ID NO: 18 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (2)..(2): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (2)..(2): CONT. FROM ABOVE: associated by a bond or through a covalent linker with a heteroatom of a side chain of an amino acid residue of the peptide sequence, or a 16-(1-hydroxy-6- oxo-1,6 -dihydropyridin-2-yl)-2,6,11-tris(1-hydroxy-6-oxo-1,6
  MOD_RES (2)..(2): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (2)..(2): CONT. FROM ABOVE: residue of the peptide sequence
  See specification as filed for detailed description of substitutions and preferred embodiments
ACDQDNPWRAYLDLLFPTDTLLLDLLW

SEQ ID NO: 19 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (2)..(2): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (2)..(2): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (2)..(2): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (2)..(2): CONT. FROM ABOVE: residue of the peptide sequence
  See specification as filed for detailed description of substitutions and preferred embodiments
AKDDQNPWRAYLDLLFPTDTLLLDLLW

SEQ ID NO: 20 - protein, 27 aa, Artificial Sequence
  Description of Artificial Sequence: Synthetic peptide
  MOD_RES (1)..(27): D-amino acid
  MOD_RES (2)..(2): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (2)..(2): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (2)..(2): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (2)..(2): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (2)..(2): CONT. FROM ABOVE: residue of the peptide sequence
  See specification as filed for detailed description of substitutions and preferred embodiments
AKDQNNPWRAYLDLLFPTDTLLLDLLW

SEQ ID NO: 21 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (27)..(27): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (27)..(27): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (27)..(27): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (27)..(27): CONT. FROM ABOVE: residue of the peptide sequence
  See specification as filed for detailed description of substitutions and preferred embodiments
ADDQNPWRAYLDLLFPTDTLLLDLLWCG

SEQ ID NO: 22 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (27)..(27): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (27)..(27): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (27)..(27): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (27)..(27): CONT. FROM ABOVE: residue of the peptide sequence
  See specification as filed for detailed description of substitutions and preferred embodiments
ADQDNPWRAYLDLLFPTDTLLLDLLWCG

SEQ ID NO: 23 - protein, 28 aa, Artificial Sequence
  Description of Artificial Sequence: Synthetic peptide
  MOD_RES (1)..(27): D-amino acid
  MOD_RES (27)..(27): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (27)..(27): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (27)..(27): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (27)..(27): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (27)..(27): CONT. FROM ABOVE: residue of the peptide sequence
  See specification as filed for detailed description of substitutions and preferred embodiments
ADDQNPWRAYLDLLFPTDTLLLDLLWKG

SEQ ID NO: 24 - protein, 29 aa, Artificial Sequence
  Description of Artificial Sequence: Synthetic peptide
  MOD_RES (1)..(28): D-amino acid
  MOD_RES (28)..(28): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (28)..(28): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (28)..(28): CONT. FROM ABOVE: associated by a bond or through a covalent linker with a heteroatom of a side chain of an amino acid residue of the peptide sequence, or a 16-(1-hydroxy-6- oxo-1,6-dihydropyridin-2-yl)-2,6,11-tris(1-hydroxy-6-oxo-1,6
  MOD_RES (28)..(28): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (28)..(28): CONT. FROM ABOVE: residue of the peptide sequence
  See specification as filed for detailed description of substitutions and preferred embodiments
AKDQNNPWRAYLDLLFPTDTLLLDLLWKG

SEQ ID NO: 25 - protein, 27 aa, Artificial Sequence
  Description of Artificial Sequence: Synthetic peptide
  MOD_RES (1)..(27): D-amino acid
  MOD_RES (2)..(2): 2,5-dioxo-1-(3,14,25-trihydroxy- 2,10,13,21,24,32-hexaoxo -3,9,14,20,25,31-hexaazatetratriacontan- 34-yl)pyrrolidin-3-yl covalently attached to the sulfur atom of the indicated D-cysteine
  See specification as filed for detailed description of substitutions and preferred embodiments
ACDDQNPWRAYLDLLFPTDTLLLDLLW

SEQ ID NO: 26 - protein, 28 aa, Artificial Sequence
  Description of Artificial Sequence: Synthetic peptide
ADQDNPWRAYLDLLFPTDTLLLDLLWCA

SEQ ID NO: 27 - protein, 28 aa, Artificial Sequence
  Description of Artificial Sequence: Synthetic peptide
ADQDNPWRAYLDLLFPTDTLLLDLLWKA

SEQ ID NO: 28 - protein, 36 aa, Artificial Sequence
  Description of Artificial Sequence: Synthetic polypeptide
GGEQNPIYWARYADWLFTTPLLLLDLALLVDADEGT

SEQ ID NO: 29 - protein, 35 aa, Artificial Sequence
  Description of Artificial Sequence: Synthetic polypeptide
AEQNPIYWARYADWLFTTPLLLLDLALLVDADEGT

SEQ ID NO: 30 - protein, 29 aa, Artificial Sequence
  Description of Artificial Sequence: Synthetic peptide
ADDQNPWRAYLDLLFPTDTLLLDLLWCKA

SEQ ID NO: 31 - protein, 32 aa, Artificial Sequence
  Description of Artificial Sequence: Synthetic polypeptide
ADDQNPWRAYLDLLFPTDTLLLDLLWDADECG

SEQ ID NO: 32 - protein, 32 aa, Artificial Sequence
  Description of Artificial Sequence: Synthetic polypeptide
ADDQNPWRAYLDLLFPTDTLLLDLLWDADEKG

SEQ ID NO: 33 - protein, 33 aa, Artificial Sequence
  Description of Artificial Sequence: Synthetic polypeptide
ADDQNPWRAYLDLLFPTDTLLLDLLWDADEKCG

SEQ ID NO: 34 - protein, 32 aa, Artificial Sequence
  Description of Artificial Sequence: Synthetic polypeptide
ACDDQNPWRAYLDLLFPTDTLLLDLLWDADEG

SEQ ID NO: 35 - protein, 32 aa, Artificial Sequence
  Description of Artificial Sequence: Synthetic polypeptide
AKDDQNPWRAYLDLLFPTDTLLLDLLWDADEG

SEQ ID NO: 36 - protein, 38 aa, Artificial Sequence
  Description of Artificial Sequence: Synthetic polypeptide
AAEQNPIYWARYADWLFTTPLLLLDLALLVDADEGTCG

SEQ ID NO: 37 - protein, 36 aa, Artificial Sequence
  Description of Artificial Sequence: Synthetic polypeptide
AEQNPIYWARYADWLFTTPLLLLDLALLVDADEGCT

SEQ ID NO: 38 - protein, 38 aa, Artificial Sequence
  Description of Artificial Sequence: Synthetic polypeptide
GGEQNPIYWARYADWLFTTPLLLLDLALLVDADEGTCG

SEQ ID NO: 39 - protein, 37 aa, Artificial Sequence
  Description of Artificial Sequence: Synthetic polypeptide
ACEQNPIYWARYADWLFTTPLLLLDLALLVDADEGTG

SEQ ID NO: 40 - protein, 36 aa, Artificial Sequence
  Description of Artificial Sequence: Synthetic polypeptide
ACEQNPIYWARYADWLFTTPLLLLDLALLVDADEGT

SEQ ID NO: 41 - protein, 36 aa, Artificial Sequence
  Description of Artificial Sequence: Synthetic polypeptide
AKEQNPIYWARYADWLFTTPLLLLDLALLVDADEGT

SEQ ID NO: 42 - protein, 37 aa, Artificial Sequence
  Description of Artificial Sequence: Synthetic polypeptide
AKEQNPIYWARYADWLFTTPLLLLDLALLVDADEGTG

SEQ ID NO: 43 - protein, 39 aa, Artificial Sequence
  Description of Artificial Sequence: Synthetic polypeptide
AAEQNPIYWARYADWLFTTPLLLLDLALLVDADEGTKCG

SEQ ID NO: 44 - protein, 36 aa, Artificial Sequence
  Description of Artificial Sequence: Synthetic polypeptide
AKEQNPIYWARYADWLFTTPLLLLDLALLVDADECT

SEQ ID NO: 45 - protein, 37 aa, Artificial Sequence
  Description of Artificial Sequence: Synthetic polypeptide
ACEQNPIYWARYANWLFTTPLLLLNLALLVDADEGTG

SEQ ID NO: 46 - protein, 36 aa, Artificial Sequence
  Description of Artificial Sequence: Synthetic polypeptide
ACEQNPIYWARYANWLFTTPLLLLNLALLVDADEGT

SEQ ID NO: 47 - protein, 37 aa, Artificial Sequence
  Description of Artificial Sequence: Synthetic polypeptide
ACEQNPIYWARYAKWLFTTPLLLLKLALLVDADEGTG

SEQ ID NO: 48 - protein, 36 aa, Artificial Sequence
  Description of Artificial Sequence: Synthetic polypeptide
GGEQNPIYWARYADWLFTTPLLLLDLALLVNANQGT

SEQ ID NO: 49 - protein, 36 aa, Artificial Sequence
  Description of Artificial Sequence: Synthetic polypeptide
AAEQNPIYWARYADWLFTTPLLLLALALLVDADEGT

SEQ ID NO: 50 - protein, 39 aa, Artificial Sequence
  Description of Artificial Sequence: Synthetic polypeptide
AAEQNPIYWARYADWLFTTPLLLLELALLVDADEGTKCG

SEQ ID NO: 51 - protein, 36 aa, Artificial Sequence
  Description of Artificial Sequence: Synthetic polypeptide
AAEQNPIYWARYAAWLFTTPLLLLDLALLVDADEGT

SEQ ID NO: 52 - protein, 36 aa, Artificial Sequence
  Description of Artificial Sequence: Synthetic polypeptide
AAEQNPIYWARYADWLFTTALLLLDLALLVDADEGT

SEQ ID NO: 53 - protein, 36 aa, Artificial Sequence
  Description of Artificial Sequence: Synthetic polypeptide
AAEQNPIYWARYADWLFTTPLLLLELALLVDADEGT

SEQ ID NO: 54 - protein, 36 aa, Artificial Sequence
  Description of Artificial Sequence: Synthetic polypeptide
AAEQNPIYWARYAEWLFTTPLLLLDLALLVDADEGT

SEQ ID NO: 55 - protein, 39 aa, Artificial Sequence
  Description of Artificial Sequence: Synthetic polypeptide
AAEQNPIIYWARYADWLFTDLPLLLLDLLALLVDADEGT

SEQ ID NO: 56 - protein, 37 aa, Artificial Sequence
  Description of Artificial Sequence: Synthetic polypeptide
GEQNPIYWAQYADWLFTTPLLLLDLALLVDADEGTCG

SEQ ID NO: 57 - protein, 38 aa, Artificial Sequence
  Description of Artificial Sequence: Synthetic polypeptide
GGEQNPIYWARYADWLFTTPLLLDLLALLVDADEGTCG

SEQ ID NO: 58 - protein, 38 aa, Artificial Sequence
  Description of Artificial Sequence: Synthetic polypeptide
GGEQNPIYWARYADWLFTTPLLLLLDALLVDADEGTCG

SEQ ID NO: 59 - protein, 38 aa, Artificial Sequence
  Description of Artificial Sequence: Synthetic polypeptide
GGEQNPIYWARYDAWLFTTPLLLLDLALLVDADEGTCG

SEQ ID NO: 60 - protein, 38 aa, Artificial Sequence
  Description of Artificial Sequence: Synthetic polypeptide
GGEQNPIYWARYAWDLFTTPLLLLDLALLVDADEGTCG

SEQ ID NO: 61 - protein, 36 aa, Artificial Sequence
  Description of Artificial Sequence: Synthetic polypeptide
AAEQNPIYWARYADWLFTTGLLLLDLALLVDADEGT

SEQ ID NO: 62 - protein, 37 aa, Artificial Sequence
  Description of Artificial Sequence: Synthetic polypeptide
DDDEDNPIYWARYADWLFTTPLLLLHGALLVDADECT

SEQ ID NO: 63 - protein, 36 aa, Artificial Sequence
  Description of Artificial Sequence: Synthetic polypeptide
DDDEDNPIYWARYADWLFTTPLLLLHGALLVDADET

SEQ ID NO: 64 - protein, 38 aa, Artificial Sequence
  Description of Artificial Sequence: Synthetic polypeptide
DDDEDNPIYWARYAHWLFTTPLLLLHGALLVDADEGCT

SEQ ID NO: 65 - protein, 37 aa, Artificial Sequence
  Description of Artificial Sequence: Synthetic polypeptide
CDDDEDNPIYWARYAHWLFTTPLLLLHGALLVDADET

SEQ ID NO: 66 - protein, 37 aa, Artificial Sequence
  Description of Artificial Sequence: Synthetic polypeptide
DDDEDNPIYWARYAHWLFTTPLLLLHGALLVDADEGT

SEQ ID NO: 67 - protein, 37 aa, Artificial Sequence
  Description of Artificial Sequence: Synthetic polypeptide
DDDEDNPIYWARYAHWLFTTPLLLLHGALLVNADECT

SEQ ID NO: 68 - protein, 37 aa, Artificial Sequence
  Description of Artificial Sequence: Synthetic polypeptide
DDDEDNPIYWARYAHWLFTTPLLLLHGALLVNADEGT

SEQ ID NO: 69 - protein, 37 aa, Artificial Sequence
  Description of Artificial Sequence: Synthetic polypeptide
DDDEDNPIYWARYAHWLFTTPLLLLHGALLVNANECT

SEQ ID NO: 70 - protein, 37 aa, Artificial Sequence
  Description of Artificial Sequence: Synthetic polypeptide
DDDEDNPIYWARYAHWLFTTPLLLLHGALLVNANEGT

SEQ ID NO: 71 - protein, 34 aa, Artificial Sequence
  Description of Artificial Sequence: Synthetic polypeptide
AEQNPIYWARYADFLFTTPLLLLDLALLVDADET

SEQ ID NO: 72 - protein, 35 aa, Artificial Sequence
  Description of Artificial Sequence: Synthetic polypeptide
AEQNPIYFARYADWLFTTPLLLLDLALLVDADEGT

SEQ ID NO: 73 - protein, 34 aa, Artificial Sequence
  Description of Artificial Sequence: Synthetic polypeptide
AEQNPIYFARYADFLFTTPLLLLDLALLWDADET

SEQ ID NO: 74 - protein, 32 aa, Artificial Sequence
  Description of Artificial Sequence: Synthetic polypeptide
AKEDQNPYWARYADWLFTTPLLLLDLALLVDG

SEQ ID NO: 75 - protein, 32 aa, Artificial Sequence
  Description of Artificial Sequence: Synthetic polypeptide
ACEDQNPYWARYADWLFTTPLLLLDLALLVDG

SEQ ID NO: 76 - protein, 32 aa, Artificial Sequence
  Description of Artificial Sequence: Synthetic polypeptide
AEDQNPYWARYADWLFTTPLLLLDLALLVDCG

SEQ ID NO: 77 - protein, 32 aa, Artificial Sequence
  Description of Artificial Sequence: Synthetic polypeptide
AEDQNPYWARYADWLFTTPLLLLELALLVECG

SEQ ID NO: 78 - protein, 32 aa, Artificial Sequence
  Description of Artificial Sequence: Synthetic polypeptide
AKEDQNDPYWARYADWLFTTPLLLLDLALLVG

SEQ ID NO: 79 - protein, 30 aa, Artificial Sequence
  Description of Artificial Sequence: Synthetic polypeptide
AKEDQNPYWRAYADLFTPLTLLDLLALWDG

SEQ ID NO: 80 - protein, 31 aa, Artificial Sequence
  Description of Artificial Sequence: Synthetic polypeptide
AEDQNPYWARYADWLFTTPLLLLELALLVCG

SEQ ID NO: 81 - protein, 30 aa, Artificial Sequence
  Description of Artificial Sequence: Synthetic polypeptide
ACEDQNPYWRAYADLFTPLTLLDLLALWDG

SEQ ID NO: 82 - protein, 27 aa, Artificial Sequence
  Description of Artificial Sequence: Synthetic peptide
ACDDQNPWRAYLDLLFPTDTLLLDLLW

SEQ ID NO: 83 - protein, 28 aa, Artificial Sequence
  Description of Artificial Sequence: Synthetic peptide
AKDDQNPWRAYLDLLFPTDTLLLDLLWC

SEQ ID NO: 84 - protein, 27 aa, Artificial Sequence
  Description of Artificial Sequence: Synthetic peptide
ACEEQNPWRAYLELLFPTETLLLELLW

SEQ ID NO: 85 - protein, 25 aa, Artificial Sequence
  Description of Artificial Sequence: Synthetic peptide
ACDDQNPWARYLDWLFPTDTLLLDL

SEQ ID NO: 86 - protein, 24 aa, Artificial Sequence
  Description of Artificial Sequence: Synthetic peptide
CDNNNPWRAYLDLLFPTDTLLLDW

SEQ ID NO: 87 - protein, 24 aa, Artificial Sequence
  Description of Artificial Sequence: Synthetic peptide
CEEQQPWAQYLELLFPTETLLLEW

SEQ ID NO: 88 - protein, 24 aa, Artificial Sequence
  Description of Artificial Sequence: Synthetic peptide
CEEQQPWRAYLELLFPTETLLLEW

SEQ ID NO: 89 - protein, 37 aa, Artificial Sequence
  Description of Artificial Sequence: Synthetic polypeptide
CDDDDDNPNYWARYANWLFTTPLLLLNGALLVEAEET

SEQ ID NO: 90 - protein, 36 aa, Artificial Sequence
  Description of Artificial Sequence: Synthetic polypeptide
CDDDDDNPNYWARYAPWLFTTPLLLLPGALLVEAEE

SEQ ID NO: 91 - protein, 34 aa, Artificial Sequence
  Description of Artificial Sequence: Synthetic polypeptide
TEDADVLLALDLLLLPTTFLWDAYRAWYPNQECA

SEQ ID NO: 92 - protein, 20 aa, Artificial Sequence
  Description of Artificial Sequence: Synthetic peptide
AEQNPIYWARYADWLFTTPL

SEQ ID NO: 93 - protein, 21 aa, Artificial Sequence
  Description of Artificial Sequence: Synthetic peptide
AEQNPIYWARYADWLFTTPCL

SEQ ID NO: 94 - protein, 21 aa, Artificial Sequence
  Description of Artificial Sequence: Synthetic peptide
ACEQNPIYWARYADWLFTTPL

SEQ ID NO: 95 - protein, 20 aa, Artificial Sequence
  Description of Artificial Sequence: Synthetic peptide
AEQNPIYFARYADWLFTTPL

SEQ ID NO: 96 - protein, 22 aa, Artificial Sequence
  Description of Artificial Sequence: Synthetic peptide
AEQNPIYFARYADLLFPTTLAW

SEQ ID NO: 97 - protein, 22 aa, Artificial Sequence
  Description of Artificial Sequence: Synthetic peptide
AEQNPIYWARYADLLFPTTLAF

SEQ ID NO: 98 - protein, 22 aa, Artificial Sequence
  Description of Artificial Sequence: Synthetic peptide
AEQNPIYWARYADLLFPTTLAW

SEQ ID NO: 99 - protein, 21 aa, Artificial Sequence
  Description of Artificial Sequence: Synthetic peptide
KEDQNPWARYADLLFPTTLAW

SEQ ID NO: 100 - protein, 20 aa, Artificial Sequence
  Description of Artificial Sequence: Synthetic peptide
KEDQNPWARYADLLFPTTLW

SEQ ID NO: 101 - protein, 22 aa, Artificial Sequence
  Description of Artificial Sequence: Synthetic peptide
ACEDQNPWARYADLLFPTTLAW

SEQ ID NO: 102 - protein, 24 aa, Artificial Sequence
  Description of Artificial Sequence: Synthetic peptide
ACEDQNPWARYADWLFPTTLLLLD

SEQ ID NO: 103 - protein, 22 aa, Artificial Sequence
  Description of Artificial Sequence: Synthetic peptide
ACEEQNPWARYAELLFPTTLAW

SEQ ID NO: 104 - protein, 24 aa, Artificial Sequence
  Description of Artificial Sequence: Synthetic peptide
ACEEQNPWARYAEWLFPTTLLLLE

SEQ ID NO: 105 - protein, 25 aa, Artificial Sequence
  Description of Artificial Sequence: Synthetic peptide
ACEEQNPWARYLEWLFPTETLLLEL

SEQ ID NO: 106 - protein, 24 aa, Artificial Sequence
  Description of Artificial Sequence: Synthetic peptide
ACEEQNPQAEYAEWLFPTTLLLLE

SEQ ID NO: 107 - protein, 21 aa, Artificial Sequence
  Description of Artificial Sequence: Synthetic peptide
WARYADWLFTTPLLLLDLALL

SEQ ID NO: 108 - protein, 21 aa, Artificial Sequence
  Description of Artificial Sequence: Synthetic peptide
YARYADWLFTTPLLLLDLALL

SEQ ID NO: 109 - protein, 21 aa, Artificial Sequence
  Description of Artificial Sequence: Synthetic peptide
WARYSDWLFTTPLLLYDLGLL

SEQ ID NO: 110 - protein, 21 aa, Artificial Sequence
  Description of Artificial Sequence: Synthetic peptide
WARYTDWFTTPLLLYDLALLA

SEQ ID NO: 111 - protein, 21 aa, Artificial Sequence
  Description of Artificial Sequence: Synthetic peptide
WARYTDWLFTTPLLLYDLGLL

SEQ ID NO: 112 - protein, 21 aa, Artificial Sequence
  Description of Artificial Sequence: Synthetic peptide
WARYADWLFTTPLLLLDLSLL

SEQ ID NO: 113 - protein, 21 aa, Artificial Sequence
  Description of Artificial Sequence: Synthetic peptide
LLALDLLLLPTTFLWDAYRAW

SEQ ID NO: 114 - protein, 21 aa, Artificial Sequence
  Description of Artificial Sequence: Synthetic peptide
LLALDLLLLPTTFLWDAYRAY

SEQ ID NO: 115 - protein, 21 aa, Artificial Sequence
  Description of Artificial Sequence: Synthetic peptide
LLGLDYLLLPTTFLWDSYRAW

SEQ ID NO: 116 - protein, 21 aa, Artificial Sequence
  Description of Artificial Sequence: Synthetic peptide
ALLALDYLLLPTTFWDTYRAW

SEQ ID NO: 117 - protein, 21 aa, Artificial Sequence
  Description of Artificial Sequence: Synthetic peptide
LLGLDYLLLPTTFLWDTYRAW

SEQ ID NO: 118 - protein, 21 aa, Artificial Sequence
  Description of Artificial Sequence: Synthetic peptide
LLSLDLLLLPTTFLWDAYRAW

SEQ ID NO: 119 - protein, 26 aa, Artificial Sequence
  Description of Artificial Sequence: Synthetic peptide
GLAGLLGLEGLLGLPLGLLEGLWLGL

SEQ ID NO: 120 - protein, 26 aa, Artificial Sequence
  Description of Artificial Sequence: Synthetic peptide
LGLWLGELLGLPLGLLGELGLLGALG

SEQ ID NO: 121 - protein, 20 aa, Artificial Sequence
  Description of Artificial Sequence: Synthetic peptide
WRAYLDLLFPTDTLLLDLLW

SEQ ID NO: 122 - protein, 20 aa, Artificial Sequence
  Description of Artificial Sequence: Synthetic peptide
WLLDLLLTDTPFLLDLYARW

SEQ ID NO: 123 - protein, 18 aa, Artificial Sequence
  Description of Artificial Sequence: Synthetic peptide
WARYLEWLFPTETLLLEL

SEQ ID NO: 124 - protein, 18 aa, Artificial Sequence
  Description of Artificial Sequence: Synthetic peptide
WAQYLELLFPTETLLLEW

SEQ ID NO: 125 - protein, 18 aa, Artificial Sequence
  Description of Artificial Sequence: Synthetic peptide
LELLLTETPFLWELYRAW

SEQ ID NO: 126 - protein, 18 aa, Artificial Sequence
  Description of Artificial Sequence: Synthetic peptide
WELLLTETPFLLELYQAW

SEQ ID NO: 127 - protein, 17 aa, Artificial Sequence
  Description of Artificial Sequence: Synthetic peptide
WLFTTPLLLLNGALLVE

SEQ ID NO: 128 - protein, 17 aa, Artificial Sequence
  Description of Artificial Sequence: Synthetic peptide
WLFTTPLLLLPGALLVE

SEQ ID NO: 129 - protein, 15 aa, Artificial Sequence
  Description of Artificial Sequence: Synthetic peptide
WARYADLLFPTTLAW

SEQ ID NO: 130 - protein, 17 aa, Artificial Sequence
  Description of Artificial Sequence: Synthetic peptide
EVLLAGNLLLLPTTFLW

SEQ ID NO: 131 - protein, 17 aa, Artificial Sequence
  Description of Artificial Sequence: Synthetic peptide
EVLLAGPLLLLPTTFLW

SEQ ID NO: 132 - protein, 15 aa, Artificial Sequence
  Description of Artificial Sequence: Synthetic peptide
WALTTPFLLDAYRAW

SEQ ID NO: 133 - protein, 24 aa, Artificial Sequence
  Description of Artificial Sequence: Synthetic peptide
NLEGFFATLGGEIALWSLVVLAIE

SEQ ID NO: 134 - protein, 22 aa, Artificial Sequence
  Description of Artificial Sequence: Synthetic peptide
EGFFATLGGEIALWSDVVLAIE

SEQ ID NO: 135 - protein, 22 aa, Artificial Sequence
  Description of Artificial Sequence: Synthetic peptide
EGFFATLGGEIPLWSDVVLAIE

SEQ ID NO: 136 - protein, 24 aa, Artificial Sequence
  Description of Artificial Sequence: Synthetic peptide
EIALVVLSWLAIEGGLTAFFGELN

SEQ ID NO: 137 - protein, 22 aa, Artificial Sequence
  Description of Artificial Sequence: Synthetic peptide
EIALVVDSWLAIEGGLTAFFGE

SEQ ID NO: 138 - protein, 22 aa, Artificial Sequence
  Description of Artificial Sequence: Synthetic peptide
EIALVVDSWLPIEGGLTAFFGE

SEQ ID NO: 139 - protein, 21 aa, Artificial Sequence
  Description of Artificial Sequence: Synthetic peptide
ILDLVFGLLFAVTSVDFLVQW

SEQ ID NO: 140 - protein, 21 aa, Artificial Sequence
  Description of Artificial Sequence: Synthetic peptide
WQVLFDVSTVAFLLGFVLDLI

SEQ ID NO: 141 - protein, 34 aa, Artificial Sequence
  Description of Artificial Sequence: Synthetic polypeptide
AEQNPIYWARYADWLFTTPLLLLDLALLVDADET

SEQ ID NO: 142 - protein, 34 aa, Artificial Sequence
  Description of Artificial Sequence: Synthetic polypeptide
AEQNPIYWARYAEWLFTTPLLLLELALLVEAEET

SEQ ID NO: 143 - protein, 33 aa, Artificial Sequence
  Description of Artificial Sequence: Synthetic polypeptide
ADDQNPWRAYLDLLFPDTTDLLLLDLLWDADET

SEQ ID NO: 144 - protein, 33 aa, Artificial Sequence
  Description of Artificial Sequence: Synthetic polypeptide
AEEQNPWRAYLELLFPETTELLLLELLWEAEET

SEQ ID NO: 145 - protein, 34 aa, Artificial Sequence
  Description of Artificial Sequence: Synthetic polypeptide
  MOD_RES (13)..(13): Gamma-carboxyglutamic acid
AEQNPIYWARYAXWLFTTPLLLLDLALLVDADET

SEQ ID NO: 146 - protein, 34 aa, Artificial Sequence
  Description of Artificial Sequence: Synthetic polypeptide
  MOD_RES (24)..(24): Gamma-carboxyglutamic acid
AEQNPIYWARYADWLFTTPLLLLXLALLVDADET

SEQ ID NO: 147 - protein, 34 aa, Artificial Sequence
  Description of Artificial Sequence: Synthetic polypeptide
  MOD_RES (13)..(13): Gamma-carboxyglutamic acid
  MOD_RES (24)..(24): Gamma-carboxyglutamic acid
AEQNPIYWARYAXWLFTTPLLLLXLALLVDADET

SEQ ID NO: 148 - protein, 34 aa, Artificial Sequence; identical to SEQ ID NO: 145
  Description of Artificial Sequence: Synthetic polypeptide
  MOD_RES (13)..(13): Alpha-aminoadipic acid

SEQ ID NO: 149 - protein, 34 aa, Artificial Sequence; identical to SEQ ID NO: 146
  Description of Artificial Sequence: Synthetic polypeptide
  MOD_RES (24)..(24): Alpha-aminoadipic acid

SEQ ID NO: 150 - protein, 34 aa, Artificial Sequence; identical to SEQ ID NO: 147
  Description of Artificial Sequence: Synthetic polypeptide
  MOD_RES (13)..(13): Alpha-aminoadipic acid
  MOD_RES (24)..(24): Alpha-aminoadipic acid

SEQ ID NO: 151 - protein, 34 aa, Artificial Sequence; identical to SEQ ID NO: 147
  Description of Artificial Sequence: Synthetic polypeptide
  MOD_RES (13)..(13): Gamma-carboxyglutamic acid
  MOD_RES (24)..(24): Alpha-aminoadipic acid

SEQ ID NO: 152 - protein, 34 aa, Artificial Sequence; identical to SEQ ID NO: 147
  Description of Artificial Sequence: Synthetic polypeptide
  MOD_RES (13)..(13): Alpha-aminoadipic acid
  MOD_RES (24)..(24): Gamma-carboxyglutamic acid

SEQ ID NO: 153 - protein, 36 aa, Artificial Sequence; identical to SEQ ID NO: 28
  Description of Artificial Sequence: Synthetic polypeptide

SEQ ID NO: 154 - protein, 36 aa, Artificial Sequence; identical to SEQ ID NO: 28
  Description of Artificial Sequence: Synthetic polypeptide

SEQ ID NO: 155 - protein, 36 aa, Artificial Sequence
  Description of Artificial Sequence: Synthetic polypeptide
AAEQNPIYWARYADWLFTTPLLLLDLALLVDADEGT

SEQ ID NO: 156 - protein, 35 aa, Artificial Sequence; identical to SEQ ID NO: 29
  Description of Artificial Sequence: Synthetic polypeptide

SEQ ID NO: 157 - protein, 35 aa, Artificial Sequence
  Description of Artificial Sequence: Synthetic polypeptide
AEQNPIYWARYANWLFTTPLLLLNLALLVDADEGT

SEQ ID NO: 158 - protein, 35 aa, Artificial Sequence
  Description of Artificial Sequence: Synthetic polypeptide
AEQNPIYWARYAKWLFTTPLLLLKLALLVDADEGT

SEQ ID NO: 159 - protein, 36 aa, Artificial Sequence; identical to SEQ ID NO: 48
  Description of Artificial Sequence: Synthetic polypeptide

SEQ ID NO: 160 - protein, 36 aa, Artificial Sequence; identical to SEQ ID NO: 49
  Description of Artificial Sequence: Synthetic polypeptide

SEQ ID NO: 161 - protein, 36 aa, Artificial Sequence; identical to SEQ ID NO: 51
  Description of Artificial Sequence: Synthetic polypeptide

SEQ ID NO: 162 - protein, 36 aa, Artificial Sequence; identical to SEQ ID NO: 52
  Description of Artificial Sequence: Synthetic polypeptide

SEQ ID NO: 163 - protein, 36 aa, Artificial Sequence; identical to SEQ ID NO: 53
  Description of Artificial Sequence: Synthetic polypeptide

SEQ ID NO: 164 - protein, 36 aa, Artificial Sequence; identical to SEQ ID NO: 54
  Description of Artificial Sequence: Synthetic polypeptide

SEQ ID NO: 165 - protein, 39 aa, Artificial Sequence; identical to SEQ ID NO: 55
  Description of Artificial Sequence: Synthetic polypeptide

SEQ ID NO: 166 - protein, 34 aa, Artificial Sequence
  Description of Artificial Sequence: Synthetic polypeptide
GEQNPIYWAQYADWLFTTPLLLLDLALLVDADEG

SEQ ID NO: 167 - protein, 35 aa, Artificial Sequence
  Description of Artificial Sequence: Synthetic polypeptide
GGEQNPIYWARYADWLFTTPLLLDLLALLVDADEG

SEQ ID NO: 168 - skipped

SEQ ID NO: 169 - protein, 35 aa, Artificial Sequence
  Description of Artificial Sequence: Synthetic polypeptide
GGEQNPIYWARYADWLFTTPLLLLLDALLVDADEG

SEQ ID NO: 170 - protein, 36 aa, Artificial Sequence
  Description of Artificial Sequence: Synthetic polypeptide
GGEQNPIYWARYDAWLFTTPLLLLDLALLVDADEGT

SEQ ID NO: 171 - protein, 35 aa, Artificial Sequence
  Description of Artificial Sequence: Synthetic polypeptide
GGEQNPIYWARYAWDLFTTPLLLLDLALLVDADEG

SEQ ID NO: 172 - protein, 36 aa, Artificial Sequence; identical to SEQ ID NO: 61
  Description of Artificial Sequence: Synthetic polypeptide

SEQ ID NO: 173 - protein, 34 aa, Artificial Sequence
  Description of Artificial Sequence: Synthetic polypeptide
DDDEDNPIYWARYADWLFTTPLLLLHGALLVDAD

SEQ ID NO: 174 - protein, 35 aa, Artificial Sequence
  Description of Artificial Sequence: Synthetic polypeptide
DDDEDNPIYWARYAHWLFTTPLLLLHGALLVDADE

SEQ ID NO: 175 - protein, 33 aa, Artificial Sequence
  Description of Artificial Sequence: Synthetic polypeptide
CEQNPIYWARYADWHFTTPLLLLDLALLVDADE

SEQ ID NO: 176 - protein, 34 aa, Artificial Sequence; identical to SEQ ID NO: 71
  Description of Artificial Sequence: Synthetic polypeptide

SEQ ID NO: 177 - protein, 33 aa, Artificial Sequence
  Description of Artificial Sequence: Synthetic polypeptide
AEQNPIYFARYADWLFTTPLLLLDLALLVDADE

SEQ ID NO: 178 - protein, 34 aa, Artificial Sequence; identical to SEQ ID NO: 73
  Description of Artificial Sequence: Synthetic polypeptide

SEQ ID NO: 179 - protein, 32 aa, Artificial Sequence
  Description of Artificial Sequence: Synthetic polypeptide
ADNNPWIYARYADLTTFPLLLLDLALLVDFDD

SEQ ID NO: 180 - protein, 32 aa, Artificial Sequence
  Description of Artificial Sequence: Synthetic polypeptide
ADNNPFIYARYADLTTWPLLLLDLALLVDFDD

SEQ ID NO: 181 - protein, 32 aa, Artificial Sequence
  Description of Artificial Sequence: Synthetic polypeptide
ADNNPFIYARYADLTTFPLLLLDLALLVDWDD

SEQ ID NO: 182 - protein, 32 aa, Artificial Sequence
  Description of Artificial Sequence: Synthetic polypeptide
ADNNPFPYARYADLTTWILLLLDLALLVDFDD

SEQ ID NO: 183 - protein, 32 aa, Artificial Sequence
  Description of Artificial Sequence: Synthetic polypeptide
ADNNPFIYAYRADLTTFPLLLLDLALLVDWDD

SEQ ID NO: 184 - protein, 32 aa, Artificial Sequence
  Description of Artificial Sequence: Synthetic polypeptide
ADNNPFIYATYADLRTFPLLLLDLALLVDWDD

SEQ ID NO: 185 - protein, 33 aa, Artificial Sequence
  Description of Artificial Sequence: Synthetic polypeptide
TEDADVLLALDLLLLPTTFLWDAYRAWYPNQEA

SEQ ID NO: 186 - protein, 30 aa, Artificial Sequence
  Description of Artificial Sequence: Synthetic polypeptide
AEDQNPYWARYADWLFTTPLLLLDLALLVD

SEQ ID NO: 187 - protein, 30 aa, Artificial Sequence
  Description of Artificial Sequence: Synthetic polypeptide
AEDQNPYWARYADWLFTTPLLLLELALLVE

SEQ ID NO: 188 - protein, 28 aa, Artificial Sequence
  Description of Artificial Sequence: Synthetic peptide
AEDQNPYWRAYADLFTPLTLLDLLALWD

SEQ ID NO: 189 - protein, 26 aa, Artificial Sequence
  Description of Artificial Sequence: Synthetic peptide
ADDQNPWRAYLDLLFPTDTLLLDLLW

SEQ ID NO: 190 - protein, 30 aa, Artificial Sequence
  Description of Artificial Sequence: Synthetic polypeptide
ADDQNPWRAYLDLLFPTDTLLLDLLWDADE

SEQ ID NO: 191 - protein, 26 aa, Artificial Sequence
  Description of Artificial Sequence: Synthetic peptide
  MOD_RES (12)..(12): Gamma-carboxyglutamic acid
ADDQNPWRAYLXLLFPTDTLLLDLLW

SEQ ID NO: 192 - protein, 26 aa, Artificial Sequence
  Description of Artificial Sequence: Synthetic peptide
  MOD_RES (18)..(18): Gamma-carboxyglutamic acid
ADDQNPWRAYLDLLFPTXTLLLDLLW

SEQ ID NO: 193 - protein, 26 aa, Artificial Sequence
  Description of Artificial Sequence: Synthetic peptide
  MOD_RES (23)..(23): Gamma-carboxyglutamic acid
ADDQNPWRAYLDLLFPTDTLLLXLLW

SEQ ID NO: 194 - protein, 26 aa, Artificial Sequence
  Description of Artificial Sequence: Synthetic peptide
  MOD_RES (12)..(12): Gamma-carboxyglutamic acid
  MOD_RES (18)..(18): Gamma-carboxyglutamic acid
ADDQNPWRAYLXLLFPTXTLLLDLLW

SEQ ID NO: 195 - protein, 26 aa, Artificial Sequence
  Description of Artificial Sequence: Synthetic peptide
  MOD_RES (12)..(12): Gamma-carboxyglutamic acid
  MOD_RES (23)..(23): Gamma-carboxyglutamic acid
ADDQNPWRAYLXLLFPTDTLLLXLLW

SEQ ID NO: 196 - protein, 26 aa, Artificial Sequence
  Description of Artificial Sequence: Synthetic peptide
  MOD_RES (18)..(18): Gamma-carboxyglutamic acid
  MOD_RES (23)..(23): Gamma-carboxyglutamic acid
ADDQNPWRAYLDLLFPTXTLLLXLLW

SEQ ID NO: 197 - protein, 26 aa, Artificial Sequence
  Description of Artificial Sequence: Synthetic peptide
  MOD_RES (12)..(12): Gamma-carboxyglutamic acid
  MOD_RES (18)..(18): Gamma-carboxyglutamic acid
  MOD_RES (23)..(23): Gamma-carboxyglutamic acid
ADDQNPWRAYLXLLFPTXTLLLXLLW

SEQ ID NO: 198 - protein, 26 aa, Artificial Sequence
  Description of Artificial Sequence: Synthetic peptide
  MOD_RES (12)..(12): Alpha-aminoadipic acid
ADDQNPWRAYLXLLFPTDTLLLDLLW

SEQ ID NO: 199 - protein, 26 aa, Artificial Sequence
  Description of Artificial Sequence: Synthetic peptide
  MOD_RES (18)..(18): Alpha-aminoadipic acid
ADDQNPWRAYLDLLFPTXTLLLDLLW

SEQ ID NO: 200 - protein, 26 aa, Artificial Sequence
  Description of Artificial Sequence: Synthetic peptide
  MOD_RES (23)..(23): Alpha-aminoadipic acid
ADDQNPWRAYLDLLFPTDTLLLXLLW

SEQ ID NO: 201 - protein, 26 aa, Artificial Sequence
  Description of Artificial Sequence: Synthetic peptide
  MOD_RES (12)..(12): Alpha-aminoadipic acid
  MOD_RES (18)..(18): Alpha-aminoadipic acid
ADDQNPWRAYLXLLFPTXTLLLDLLW

SEQ ID NO: 202 - protein, 26 aa, Artificial Sequence
  Description of Artificial Sequence: Synthetic peptide
  MOD_RES (12)..(12): Alpha-aminoadipic acid
  MOD_RES (23)..(23): Alpha-aminoadipic acid
ADDQNPWRAYLXLLFPTDTLLLXLLW

SEQ ID NO: 203 - protein, 26 aa, Artificial Sequence
  Description of Artificial Sequence: Synthetic peptide
  MOD_RES (18)..(18): Alpha-aminoadipic acid
  MOD_RES (23)..(23): Alpha-aminoadipic acid
ADDQNPWRAYLDLLFPTXTLLLXLLW

SEQ ID NO: 204 - protein, 26 aa, Artificial Sequence
  Description of Artificial Sequence: Synthetic peptide
  MOD_RES (12)..(12): Alpha-aminoadipic acid
  MOD_RES (18)..(18): Alpha-aminoadipic acid
  MOD_RES (23)..(23): Alpha-aminoadipic acid
ADDQNPWRAYLXLLFPTXTLLLXLLW

SEQ ID NO: 205 - protein, 26 aa, Artificial Sequence
  Description of Artificial Sequence: Synthetic peptide
  MOD_RES (12)..(12): Gamma-carboxyglutamic acid
  MOD_RES (18)..(18): Alpha-aminoadipic acid
ADDQNPWRAYLXLLFPTXTLLLDLLW

SEQ ID NO: 206 - protein, 26 aa, Artificial Sequence
  Description of Artificial Sequence: Synthetic peptide
  MOD_RES (12)..(12): Gamma-carboxyglutamic acid
  MOD_RES (23)..(23): Alpha-aminoadipic acid
ADDQNPWRAYLXLLFPTDTLLLXLLW

SEQ ID NO: 207 - protein, 26 aa, Artificial Sequence
  Description of Artificial Sequence: Synthetic peptide
  MOD_RES (12)..(12): Gamma-carboxyglutamic acid
  MOD_RES (18)..(18): Gamma-carboxyglutamic acid
  MOD_RES (23)..(23): Alpha-aminoadipic acid
ADDQNPWRAYLXLLFPTXTLLLXLLW

SEQ ID NO: 208 - protein, 26 aa, Artificial Sequence
  Description of Artificial Sequence: Synthetic peptide
  MOD_RES (12)..(12): Alpha-aminoadipic acid
  MOD_RES (18)..(18): Gamma-carboxyglutamic acid
ADDQNPWRAYLXLLFPTXTLLLDLLW

SEQ ID NO: 209 - protein, 26 aa, Artificial Sequence
  Description of Artificial Sequence: Synthetic peptide
  MOD_RES (12)..(12): Alpha-aminoadipic acid
  MOD_RES (23)..(23): Gamma-carboxyglutamic acid
ADDQNPWRAYLXLLFPTDTLLLXLLW

SEQ ID NO: 210 - protein, 26 aa, Artificial Sequence
  Description of Artificial Sequence: Synthetic peptide
  MOD_RES (12)..(12): Gamma-carboxyglutamic acid
  MOD_RES (18)..(18): Alpha-aminoadipic acid
  MOD_RES (23)..(23): Gamma-carboxyglutamic acid
ADDQNPWRAYLXLLFPTXTLLLXLLW

SEQ ID NO: 211 - protein, 29 aa, Artificial Sequence
  Description of Artificial Sequence: Synthetic peptide
GEEQNPWLGAYLDLLFPLELLGLLELGLW

SEQ ID NO: 212 - protein, 28 aa, Artificial Sequence
  Description of Artificial Sequence: Synthetic peptide
ADDDDDDPWQAYLDLLFPTDTLLLDLLW

SEQ ID NO: 213 - protein, 26 aa, Artificial Sequence
  Description of Artificial Sequence: Synthetic peptide
AEEQNPWRAYLELLFPTETLLLELLW

SEQ ID NO: 214 - protein, 24 aa, Artificial Sequence
  Description of Artificial Sequence: Synthetic peptide
ADDQNPWARYLDWLFPTDTLLLDL

SEQ ID NO: 215 - protein, 23 aa, Artificial Sequence
  Description of Artificial Sequence: Synthetic peptide
DNNNPWRAYLDLLFPTDTLLLDW

SEQ ID NO: 216 - protein, 24 aa, Artificial Sequence
  Description of Artificial Sequence: Synthetic peptide
AEEQNPWARYLEWLFPTETLLLEL

SEQ ID NO: 217 - protein, 25 aa, Artificial Sequence
  Description of Artificial Sequence: Synthetic peptide
DDDDDDPWQAYLDLFPTDTLALDLW

SEQ ID NO: 218 - protein, 23 aa, Artificial Sequence
  Description of Artificial Sequence: Synthetic peptide
EEQQPWAQYLELLFPTETLLLEW

SEQ ID NO: 219 - protein, 23 aa, Artificial Sequence
  Description of Artificial Sequence: Synthetic peptide
EEQQPWRAYLELLFPTETLLLEW

SEQ ID NO: 220 - protein, 23 aa, Artificial Sequence
  Description of Artificial Sequence: Synthetic peptide
AEDQNPWARYADWLFPTTLLLLD

SEQ ID NO: 221 - protein, 23 aa, Artificial Sequence
  Description of Artificial Sequence: Synthetic peptide
AEEQNPWARYAEWLFPTTLLLLE

SEQ ID NO: 222 - protein, 21 aa, Artificial Sequence
  Description of Artificial Sequence: Synthetic peptide
AEDQNPWARYADLLFPTTLAW

SEQ ID NO: 223 - protein, 21 aa, Artificial Sequence
  Description of Artificial Sequence: Synthetic peptide
AEEQNPWARYAELLFPTTLAW

SEQ ID NO: 224 - protein, 36 aa, Artificial Sequence
  Description of Artificial Sequence: Synthetic polypeptide
DDDDDNPNYWARYANWLFTTPLLLLNGALLVEAEET

SEQ ID NO: 225 - protein, 36 aa, Artificial Sequence
  Description of Artificial Sequence: Synthetic polypeptide
DDDDDNPNYWARYAPWLFTTPLLLLPGALLVEAEET

SEQ ID NO: 226 - protein, 34 aa, Artificial Sequence; identical to SEQ ID NO: 73
  Description of Artificial Sequence: Synthetic polypeptide

SEQ ID NO: 227 - protein, 34 aa, Artificial Sequence; identical to SEQ ID NO: 71
  Description of Artificial Sequence: Synthetic polypeptide

SEQ ID NO: 228 - protein, 20 aa, Artificial Sequence
  Description of Artificial Sequence: Synthetic peptide
AEQNPIYWARYADWLFTTPL

SEQ ID NO: 229 - protein, 22 aa, Artificial Sequence
  Description of Artificial Sequence: Synthetic peptide
AEQNPIYFARYADLLFPTTLAW

SEQ ID NO: 230 - protein, 22 aa, Artificial Sequence
  Description of Artificial Sequence: Synthetic peptide
AEQNPIYWARYADLLFPTTLAF

SEQ ID NO: 231 - protein, 22 aa, Artificial Sequence
  Description of Artificial Sequence: Synthetic peptide
AEQNPIYWARYADLLFPTTLAW

SEQ ID NO: 232 - protein, 20 aa, Artificial Sequence
  Description of Artificial Sequence: Synthetic peptide
AEQNPIYFARYADWLFTTPL

SEQ ID NO: 233 - protein, 20 aa, Artificial Sequence
  Description of Artificial Sequence: Synthetic peptide
EDQNPWARYADLLFPTTLAW

SEQ ID NO: 234 - protein, 34 aa, Artificial Sequence
  Description of Artificial Sequence: Synthetic polypeptide
GLAGLAGLLGLEGLLGLPLGLLEGLWLGLELEGN

SEQ ID NO: 235 - protein, 32 aa, Artificial Sequence
  Description of Artificial Sequence: Synthetic polypeptide
EQNPIYILDLVFGLLFAVTSVDFLVQWDDAGD

SEQ ID NO: 236 - protein, 24 aa, Artificial Sequence
  Description of Artificial Sequence: Synthetic peptide
NLEGFFATLGGEIALWSLVVLAIE

SEQ ID NO: 237 - protein, 24 aa, Artificial Sequence
  Description of Artificial Sequence: Synthetic peptide
NNEGFFATLGGEIALWSDVVLAIE

SEQ ID NO: 238 - protein, 25 aa, Artificial Sequence
  Description of Artificial Sequence: Synthetic peptide
DNNEGFFATLGGEIPLWSDVVLAIE

SEQ ID NO: 239 - protein, 28 aa, Artificial Sequence
  Description of Artificial Sequence: Synthetic peptide
  MOD_RES (1)..(28): D-amino acid
AKDDQNPWRAYLDLLFPTDTLLLDLLWA

SEQ ID NO: 240 - protein, 28 aa, Artificial Sequence
  Description of Artificial Sequence: Synthetic peptide
  MOD_RES (1)..(28): D-amino acid
  MOD_RES (2)..(2): a (3,14,25-trihydroxy-2,10,13,21,24-pentaoxo- 3,9,14,20,25-pentaazatriacontan-30-yl)amino group covalently associated by a bond or through a covalent linker with a heteroatom of a side chain of an amino acid residue of the peptide sequence, a
  MOD_RES (2)..(2): CONT. FROM ABOVE: (3,14,25,36-tetrahydroxy- 2,10,13,21,24,32,35-heptaoxo-3,9,14,20,25,31,36- heptaazahentetracontan-41-yl)amino group covalently associated by a bond or through a covalent linker with a heteroatom of a side chain of an amino acid
  MOD_RES (2)..(2): CONT. FROM ABOVE: residue of the peptide sequence, or a 16-(1-hydroxy-6-oxo-1,6-dihydropyridin-2-yl)- 2,6,11-tris(1-hydroxy-6-oxo-1,6-dihydropyridine-2-carbonyl)-16- oxo-2,6,11,15 -tetraazahexadecyl group covalently associated by a bond or
  MOD_RES (2)..(2): CONT. FROM ABOVE: through a covalent linker with a heteroatom of a side chain of an amino acid residue of the peptide sequence
  See specification as filed for detailed description of substitutions and preferred embodiments
AKDDQNPWRAYLDLLFPTDTLLLDLLWA

SEQ ID NO: 241 - protein, 28 aa, Artificial Sequence
  Description of Artificial Sequence: Synthetic peptide
  MOD_RES (1)..(28): D-amino acid
  MOD_RES (2)..(2): a (3,14,25-trihydroxy-2,10,13,21,24-pentaoxo- 3,9,14,20,25-pentaazatriacontan-30-yl)amino group chelating a radionuclide where the group is covalently associated by a bond or through a covalent linker with a heteroatom of a side chain of an amino
  MOD_RES (2)..(2): CONT. FROM ABOVE: acid residue of the peptide sequence, a (3,14,25,36-tetrahydroxy-2,10,13,21,24,32,35-heptaoxo- 3,9,14,20,25,31,36-heptaazahentetracontan-41-yl)amino group chelating a radionuclide where the group is covalently
  MOD_RES (2)..(2): CONT. FROM ABOVE: associated by a bond or through a covalent linker with a heteroatom of a side chain of an amino acid residue of the peptide sequence, or a 16-(1-hydroxy-6- oxo-1,6 -dihydropyridin-2-yl)-2,6,11-tris(1-hydroxy-6-oxo-1,6
  MOD_RES (2)..(2): CONT. FROM ABOVE: -dihydropyridine-2-carbonyl)- 16-oxo-2,6,11,15-tetraazahexadecyl chelating a radionuclide where the group is group covalently associated by a bond or through a covalent linker with a heteroatom of a side chain of an amino acid
  MOD_RES (2)..(2): CONT. FROM ABOVE: residue of the peptide sequence
  See specification as filed for detailed description of substitutions and preferred embodiments
AKDDQNPWRAYLDLLFPTDTLLLDLLWA

SEQ ID NO: 242 - protein, 28 aa, Artificial Sequence
  Description of Artificial Sequence: Synthetic peptide
  MOD_RES (1)..(28): D-amino acid
  MOD_RES (2)..(2): 3,4-dioxo-2-((3,14,25-trihydroxy-2,10,13,21,24- pentaoxo-3,9,14,20,25-pentaazatriacontan-30-yl)amino)cyclobut-1- en-1-yl covalently attached to the epsilon-nitrogen atom of the indicated D-lysine, (4-(1-(1-hydroxy-6-oxo-1,6-dihydropyridin-2
  MOD_RES (2)..(2): CONT. FROM ABOVE: -yl)-6,11,15-tris(1-hydroxy- 6-oxo-1,6-dihydropyridine-2-carbonyl)-1-oxo-2,6,11,15- tetraazaheptadecan-17-yl)phenyl)carbamothioyl covalently attached to the epsilon-nitrogen atom of the indicated D-lysine, (4-(3-(3,14,25,36
  MOD_RES (2)..(2): CONT. FROM ABOVE: -tetrahydroxy -2,10,13,21,24, 32,35-heptaoxo-3,9,14,20,25,31,36-heptaazahentetracontan-41-yl) thioureido) phenyl)carbamothioyl covalently attached to the epsilon-nitrogen atom of the indicated D-lysine, or (4-(3-(3,14, 25-trihydroxy
  MOD_RES (2)..(2): CONT. FROM ABOVE: -2,10,13,21,24-pentaoxo-3,9, 14,20,25-pentaazatriacontan-30-yl)thioureido)phenyl)carbamothioyl covalently attached to the epsilon-nitrogen atom of the indicated D-lysine
  See specification as filed for detailed description of substitutions and preferred embodiments
AKDDQNPWRAYLDLLFPTDTLLLDLLWA

SEQ ID NO: 243 - protein, 37 aa, Artificial Sequence; identical to SEQ ID NO: 42
  Description of Artificial Sequence: Synthetic polypeptide
  MOD_RES (1)..(34): D-amino acid
  MOD_RES (36)..(36): D-amino acid

SEQ ID NO: 244 - protein, 37 aa, Artificial Sequence; identical to SEQ ID NO: 42
  Description of Artificial Sequence: Synthetic polypeptide
  MOD_RES (1)..(34): D-amino acid
  MOD_RES (2)..(2): (4-(3-(3,14,25-trihydroxy-2,10,13,21,24- pentaoxo-3,9,14,20,25 -pentaazatriacontan-30-yl)thioureido)phenyl) carbamothioyl covalently attached to the epsilon-nitrogen atom of the indicated D-lysine
  MOD_RES (36)..(36): D-amino acid
  See specification as filed for detailed description of substitutions and preferred embodiments